(19) United States
(12) Patent Application Publication
(10) Pub. No.: US 2018/0042922 A1
(43) Pub. Date: Feb. 15, 2018

(54) COMPOSITIONS AND METHODS OF TREATING A NEURODEGENERATIVE DISEASE

(71) Applicant: Axovant Sciences GmbH, Basel (CH)

(72) Inventors: Pavan Kumar CHERUVU, New York, NY (US); Kunal KISHNANI, Flushing, NY (US); Bryan M. LEWIS, North Brunswick, NJ (US); Stephen Clement PISCITELLI, Hillsborough, NC (US); Shankar RAMASWAMY, Cincinnati, OH (US); Gregory M. WEINHOFF, New York, NY (US); Ebenezer ASARE, New Haven, CT (US)

(21) Appl. No.: 15/677,837

(22) Filed: Aug. 15, 2017

Related U.S. Application Data
(60) Provisional application No. 62/375,355, filed on Aug. 15, 2016.
(60) Provisional application No. 62/522,988, filed on Jun. 21, 2017.
(60) Provisional application No. 62/532,846, filed on Jul. 14, 2017.
(60) Provisional application No. 62/543,685, filed on Aug. 10, 2017.

(51) Int. Cl. A61K 31/496; A61K 9/00; A61K 31/46; A61K 31/13; A61K 31/445

(52) U.S. Cl. CPC A61K 31/496; A61K 31/13; A61K 31/445; A61K 31/46; A61K 9/0053

(57) ABSTRACT

The present application relates to novel compositions useful for the treatment of a neurodegenerative disease and uses thereof. The present application also relates to novel compositions and methods for the reduction of side effects in a subject being treated for a neurodegenerative disease. The present application also relates to novel compositions and methods for enhancing the standard of care of the treatment of a neurodegenerative disease. The present application also relates to novel compositions and methods for enhancing the efficacy of one or more treatments for a neurodegenerative disease.

### CROSS REFERENCE TO RELATED APPLICATIONS

This application claims the priority benefit under 35 U.S.C. 119(e) of U.S. Provisional Application No. 62/375,355 filed Aug. 15, 2016, U.S. Provisional Application No. 62/522,988, filed Jun. 21, 2017, U.S. Provisional Application No. 62/532,846 filed Jul. 14, 2017, and U.S. Provisional Application No. 62/543,685 filed Aug. 10, 2017, the disclosures of which are incorporated by reference in their entirety.

### FIELD

The disclosure generally refers to compositions comprising a 5-HT₆ receptor antagonist, a 5-HT_{2A} inverse agonist, a NMDA receptor antagonist, or combinations thereof, in combination with an acetylcholinesterase inhibitor and an anti-cholinergic agent, and the use of the compositions to treat neurodegenerative disease in a subject in need thereof.

### BACKGROUND

Acetylcholinesterase inhibitors are widely prescribed for the treatment of neurological diseases, for example, Alzheimer's disease, and are thought to result in therapeutic benefit by the inhibition of acetylcholinesterase, thereby increasing the concentration of acetylcholine in synapses in the central nervous system. However, increasing acetylcholine in periphery neural pathways can cause adverse effects including nausea, vomiting, and diarrhea. Thus, compositions and methods for treating neurodegenerative diseases that can reduce or eliminate the adverse effects of acetylcholinesterase inhibitors while maintaining or enhancing the therapeutic benefits to the central nervous system are desired.

### SUMMARY

Embodiments herein are directed to compositions and methods for the treatment of neurodegenerative diseases. In some embodiments, the composition comprises a 5-HT₆ receptor antagonist, a 5-HT_{2A} inverse agonist, a NMDA receptor antagonist, or combinations thereof, in combination with an acetylcholinesterase inhibitor and an anti-cholinergic agent. In some embodiments, the composition comprises at least one pharmaceutically acceptable excipient. In some embodiments, the composition comprises one or more additional therapeutic agents. In some embodiments, the composition is suitable for oral administration.

In some embodiments, the composition comprises a 5-HT₆ receptor antagonist, an acetylcholinesterase inhibitor, and an anti-cholinergic agent. In some embodiments, the composition comprises a 5-HT_{2A} inverse agonist, an acetylcholinesterase inhibitor, and an anti-cholinergic agent. In some embodiments, the composition comprises a NMDA receptor antagonist, an acetylcholinesterase inhibitor, and an anti-cholinergic agent.

In some embodiments, the composition comprises a 5-HT₆ receptor antagonist, a 5-HT_{2A} inverse agonist, an acetylcholinesterase inhibitor, and an anti-cholinergic agent. In some embodiments, the composition comprises a 5-HT_{2A} inverse agonist, a NMDA receptor antagonist, an acetylcholinesterase inhibitor, and an anti-cholinergic agent. In some embodiments, the composition comprises a 5-HT_{2A} inverse agonist, a NMDA receptor antagonist, an acetylcholinesterase inhibitor, and an anti-cholinergic agent.

In some embodiments, the composition comprises a 5-HT₆ receptor antagonist, a 5-HT_{2A} inverse agonist, and a NMDA receptor antagonist. In some embodiments, the composition comprises a 5-HT₆ receptor antagonist, a 5-HT_{2A} inverse agonist, a NMDA receptor antagonist and an acetylcholinesterase inhibitor. In some embodiments, the composition comprises a 5-HT₆ receptor antagonist, a 5-HT_{2A} inverse agonist, a NMDA receptor antagonist, an acetylcholinesterase inhibitor, and an anti-cholinergic agent.

In some embodiments, the 5-HT₆ receptor antagonist is a therapeutically effective amount of 3-phenylsulfonyl-8-piperazinyl-1yl-quinoline or pharmaceutically acceptable salts, hydrates, polymorphs, or solvates thereof. In some embodiments, the therapeutically effective amount of 3-phenylsulfonyl-8-piperazinyl-1yl-quinoline or pharmaceutically acceptable salts, hydrates, polymorphs, or solvates thereof is from about 0.001 mg to about 1,000 mg, about 0.001 mg to about 200 mg, about 0.001 mg to about 175 mg, or 0.001 mg to about 70 mg. In some embodiments, the therapeutically effective amount of 3-phenylsulfonyl-8-piperazinyl-1yl-quinoline or pharmaceutically acceptable salts, hydrates, polymorphs, or solvates thereof is about 15 mg, about 35 mg, or about 70 mg. In some embodiments, the therapeutically effective amount of 3-phenylsulfonyl-8-piperazinyl-1yl-quinoline or pharmaceutically acceptable salts, hydrates, polymorphs, or solvates thereof is an amount selected from the group consisting of an amount of 3-phenylsulfonyl-8-piperazinyl-1yl-quinoline that may cause convulsions in a subject to which it is administered; an amount that would be expected to exceed the maximum tolerated dose for the subject to which it is administered; an amount associated with systemic exposures characterized by an AUCₜₐᵤ₋ₛₛ of about 8.2 μg·h/ml, a Cₘₐₓ of about 0.26 μg/ml; or a combination thereof an mount associated with systemic exposures characterized by an AUC, Cₘₐₓ, or combinations thereof, that are about 2 to about 3 times higher than the mean clinical exposure achieved at the proposed clinical dose for monotherapy with 3-phenylsulfonyl-8-piperazinyl-1yl-quinoline (i.e. mean AUCₜₐᵤ₋ₛₛ of about 3.2 μg·h/ml and Cₘₐₓ of about 0.180 μg/ml), an amount associated with a recorded systemic clinical exposure that is greater than the highest recorded systemic clinical exposure (AUC_{0-∞} of about 9.25 μg·h/ml and Cₘₐₓ of about 0.293 μg/ml), an amount of 3-phenylsulfonyl-8-piperazinyl-1yl-quinoline that is greater than about 10 mg/kg/day, an amount of 3-phenylsulfonyl-8-piperazinyl-1yl-quinoline that is greater than 15 mg/day, a dose of 3-phenylsulfonyl-8-piperazinyl-1yl-quinoline that is greater than about 35 mg/day or any combination thereof.

In some embodiments, the 5-HT_{2A} inverse agonist is a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates, polymorphs, or solvates thereof. In some embodiments, the therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates, polymorphs, or solvates thereof is from about 0.001 mg to about 1,000 mg, or about 0.001 mg to about 100 mg. In some embodiments, 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof is administered to a subject in need thereof in an amount that is considered to sub therapeutic. In some embodiments, the therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates, polymorphs, or solvates thereof is about 20 mg, about 40 mg, or about 80 mg.

In some embodiments, the NMDA receptor antagonist is a therapeutically effective amount of memantine, amantadine, and ketamine or pharmaceutically acceptable salts, hydrates, polymorphs, or solvates thereof. In some embodiments, the NMDA receptor antagonist is a therapeutically effective amount of memantine or pharmaceutically acceptable salts, hydrates, polymorphs, or solvates thereof. In some embodiments, the therapeutically effective amount of memantine or pharmaceutically acceptable salts, hydrates, polymorphs, or solvates thereof is from about 0.001 mg to about 1,000 mg, or about 0.001 mg to about 30 mg. In some embodiments, the therapeutically effective amount of memantine or pharmaceutically acceptable salts, hydrates, polymorphs, or solvates thereof is about 5 mg, about 7 mg, about 10 mg, about 14 mg, about 20 mg, about 21 mg, or about 28 mg.

In some embodiments, the acetylcholinesterase inhibitor is selected from the group consisting of donepezil, rivastigmine, galantamine, tacrine, physostigmine, pyridostigmine, neostigmine, icopezil, zanapezil, ipidacrine, phenserine, ambenonium, edrophonium, ladostigil, huperzine A, or pharmaceutically acceptable salts, hydrates, polymorphs, or solvates thereof. In some embodiments, the acetylcholinesterase inhibitor is donepezil or pharmaceutically acceptable salts, hydrates, polymorphs, or solvates thereof. In some embodiments, the therapeutically effective amount of donepezil or pharmaceutically acceptable salts, hydrates, polymorphs, or solvates thereof is configured for immediate release, extended release, delayed release, or any combination thereof. In some embodiments, the therapeutically effective amount of donepezil or pharmaceutically acceptable salts, hydrates, polymorphs, or solvates thereof is from about 0.001 mg to about 1,000 mg, about 0.001 mg to about 30 mg, or about 0.2 mg to about 138 mg. In some embodiments, the therapeutically effective amount of donepezil or pharmaceutically acceptable salts, hydrates, polymorphs, or solvates thereof is about 5 mg, 10 mg, or 23 mg.

In some embodiments, the anti-cholinergic agent is selected from the group consisting of quaternary ammonium anti-cholinergic muscarinic receptor antagonist, a quaternary ammonium non-selective peripheral Anti-Cholinergic agent, a sulfonium non-selective peripheral Anti-Cholinergic agent, a non-selective peripheral muscarinic anti-cholinergic agent, (1 S)-(3R)-1-azabicyclo[2.2.2]oct-3-yl 3,4-dihydro-1-phenyl-2(1H)-iso-quinolinecarboxylate (solifenacin) and its pharmaceutically acceptable salts, 1-methylpiperidin-4-yl) 2,2-di(phenyl)-2-propoxyacetate (propiverine) and its pharmaceutically acceptable salts, 1,4,5, 6-tetrahydro-1-methylpyrimidin-2-ylmethyl α-cyclohexyl-α-hydroxy-α-phenylacetate (oxyphencyclimine) and its pharmaceutically acceptable salts, (R)-N,N-diisopropyl-3-(2-hydroxy-5-methylphenyl)-3-phenylpropanamine (tolterodine) and its pharmaceutically acceptable salts. In some embodiments, the quaternary ammonium anti-cholinergic muscarinic receptor antagonist is selected from trospium and glycopyrrolate or pharmaceutically acceptable salts, hydrates or solvates thereof. In some embodiments, the quaternary ammonium anti-cholinergic muscarinic receptor antagonist is trospium or pharmaceutically acceptable salts, hydrates or solvates thereof. In some embodiments, the quaternary ammonium anti-cholinergic muscarinic receptor antagonist is trospium chloride. In some embodiments, the therapeutically effective amount of trospium or pharmaceutically acceptable salts, hydrates or solvates thereof is from about 0.1 mg to about 120 mg. In some embodiments, the therapeutically effective amount of trospium or pharmaceutically acceptable salts, hydrates or solvates thereof is about 20 mg, about 40 mg, or about 60 mg. In some embodiments, the therapeutically effective amount of trospium or pharmaceutically acceptable salts, hydrates or solvates thereof is about 20 mg per day, about 40 mg per day, or about 60 mg per day. In some embodiments, the therapeutically effective amount of tropsium is an amount from about 20% to about 600% of the amount of trospium or pharmaceutically acceptable salts, hydrates or solvates thereof that is currently administered for anti-cholinergic therapy.

Some embodiments are directed to methods of treating a neurodegenerative disease in a patient in need thereof comprising administering to the patient any of the compositions described herein.

Some embodiments are directed to methods for enhancing the standard of care in a subject being treated for a neurodegenerative disease comprising administering to a patient any of the composition described herein. In some embodiments, enhancing the standard of care includes enhancing the efficacy and enhancing safety of the standard of care or a combination thereof

Some embodiments are directed to methods for enhancing the efficacy of a treatment without causing cholinergic toxicity or cholinergic side effects in a subject being treated for a neurodegenerative disease comprising administering to a patient any of the compositions described herein.

Some embodiments are directed to methods for reducing side effects in a subject being treated for a neurodegenerative disease comprising administering to a patient a composition described herein. Some embodiments are directed to methods of reducing side effects including side effects associated with cholinesterase inhibitors, in a subject being treated for a neurodegenerative disease.

In some embodiments, an efficacious therapeutically effective amount of any of the compounds or agents described herein is administered to the patient either orally, intravenously or parenterally. In some embodiments, any of the compounds or agents described herein are administered once a day, twice a day, three times a day, or four times a day. In some embodiments, any of the compounds or agents described herein can be administered concurrently. In some embodiments, the therapeutically effective amount of trospium is administered in an extended release formulation. In some embodiments, the trospium is trospium chloride.

### BRIEF DESCRIPTION OF THE FIGURES

FIG. 1 shows self-reported nausea (VAS score; 0-100 mm) at various time points post dosing on Day 10.

FIG. 2 shows REM density data across placebo and treatment conditions.

FIG. 3 shows percentage of stage R data across placebo and treatment conditions.

### DETAILED DESCRIPTION

The present application relates to novel compositions useful for the treatment of a neurodegenerative disease and uses thereof. The present application also relates to novel compositions and methods for the reduction of side effects in a subject being treated for a neurodegenerative disease. The present application also relates to novel compositions and methods for enhancing the standard of care of the treatment of a neurodegenerative disease. The present application also relates to novel compositions and methods for enhancing the efficacy of one or more treatments for a neurodegenerative disease.

In some embodiments, the compounds for use in the methods described herein may be formulated as pharmaceutical compositions. Pharmaceutical compositions of this disclosure may comprise the compounds described herein or a pharmaceutically acceptable salt thereof and a pharmaceutically acceptable carrier. Such compositions may optionally comprise at least one additional therapeutic agent useful for treating a neurodegenerative disease.

The compounds of this disclosure may be employed in a conventional manner for controlling the disease described herein, including, but not limited to, a neurodegenerative disease, and for treating diseases or reducing the advancement or severity of side effects. Such methods of treatment, their dosage levels and requirements may be selected by those of ordinary skill in the art from available methods and techniques. For example, the compounds of this disclosure may be combined with a pharmaceutically acceptable adjuvant for administration to a patient suffering from a neurodegenerative disease in a pharmaceutically acceptable manner and in an amount effective to lessen the severity of that disease.

Alternatively, the compounds of this disclosure may be used in compositions and methods for treating or protecting individuals against the diseases described herein, including but not limited to a neurodegenerative disease, over extended periods of time. The compounds may be employed in such compositions either alone or together with other compounds of this disclosure in a manner consistent with the conventional utilization of such compounds in pharmaceutical compositions. For example, a compound of this disclosure may be combined with pharmaceutically acceptable adjuvants conventionally employed in vaccines and administered in prophylactically effective amounts to protect individuals over an extended period of time against the diseases described herein, including, but not limited to, neurodegenerative diseases.

In each of the embodiments disclosed herein, the compounds and methods can be utilized with or on a subject in need of such treatment, which can also be referred to as “in need thereof” As used herein, the phrase “in need thereof” means that the subject has been identified as having a need for the particular method or treatment and that the treatment has been given to the subject for that particular purpose.

The term “patient” and “subject” are interchangeable and may be taken to mean any living organism which may be treated with compounds of the present disclosure. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human. In some embodiments, the subject is a human aged 55 years or older.

As used herein, the terms “combination,” “combined,” and related terms refer to the simultaneous or sequential administration of therapeutic agents in accordance with this disclosure. For example, a described compound may be administered with another therapeutic agent simultaneously or sequentially in separate unit dosage forms or together in a single unit dosage form. Accordingly, the present disclosure provides a single unit dosage form comprising a described compound, an additional therapeutic agent, and a pharmaceutically acceptable carrier, adjuvant, or vehicle. Two or more agents are typically considered to be administered “in combination” when a patient or individual is simultaneously exposed to both agents. In many embodiments, two or more agents are considered to be administered “in combination” when a patient or individual simultaneously shows therapeutically relevant levels of the agents in a particular target tissue or sample (e.g., in brain, in serum, etc.).

The term “pharmaceutically acceptable excipient” refers to a non-toxic excipient that may be administered to a patient, together with a compound of this disclosure, and which does not destroy the pharmacological activity thereof. Pharmaceutically acceptable excipients that may be used in these compositions include, but are not limited to, ion exchangers, alumina, aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Pharmaceutically acceptable excipients that may be used in the pharmaceutical compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, wool fat and self-emulsifying drug delivery systems (SEDDS) such as α-tocopherol, polyethyleneglycol 1000 succinate, or other similar polymeric delivery matrices.

The term “therapeutically effective amount” as used herein refers to the amount of active compound or pharmaceutical agent that elicits the biological or medicinal response in a tissue, system, animal, individual or human that is being sought by a researcher, veterinarian, medical doctor or other clinician, which includes one or more of the following: (1) Preventing the disease; for example, preventing a disease, condition or disorder in an individual that may be predisposed to the disease, condition or disorder but does not yet experience or display the pathology or symptomatology of the disease, (2) Inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) Ameliorating the disease; for example, ameliorating a disease, condition or disorder in an individual that is experiencing or displaying the pathology or symptomatology of the disease, condition or disorder (i.e., reversing the pathology and/or symptomatology). In some embodiments, the therapeutically effective amount of a compound represents the daily dose a particular compound. In some embodiments, the daily dose of a particular compound may be administered as a single daily dose or may be divided into two or more doses of equal or unequal amounts administered throughout the day. In some embodiments, a therapeutically effective amount may be a dose of a compound that results in the reduction, or elimination of the side effects caused by the administration of another compound.

The term “sub therapeutic amount” as used herein refers to a dosage that is below that typically used for the subject agent in typical therapeutic or prophylactic use.

As used herein, “fixed-dose-combination or FDC” refers to a combination of two drugs or active ingredients presented in a single dosage unit such as a tablet or oral dosage form. When formulating a solid fixed dose combination, the objective is to provide a patient-convenient combination dosage form of active ingredients that is bioequivalent to the corresponding free-combination of the same active ingredients.

The scientific literature that has evolved around receptors has adopted a number of terms to refer to ligands having various effects on receptors. For clarity and consistency, the following definitions will be used throughout this patent document.

“Agonists” shall mean moieties that interact and activate the receptor, such as the 5-HT_{2A} receptor, and initiate a physiological or pharmacological response characteristic of that receptor. For example, when moieties activate the intracellular response upon binding to the receptor, or enhance GTP binding to membranes.

The term “antagonists” is intended to mean moieties that competitively bind to the receptor at the same site as agonists (for example, the endogenous ligand), but which do not activate the intracellular response initiated by the active form of the receptor, and can thereby inhibit the intracellular responses by agonists or partial agonists. Antagonists do not diminish the baseline intracellular response in the absence of an agonist or partial agonist.

Alkyl groups, whether alone or as part of another group, may be straight chain or branched and the groups alkoxy and alkanoyl shall be interpreted similarly. Alkyl moieties are more preferably C1-4 alkyl, e.g. methyl or ethyl. The term ‘halogen’ is used herein to describe, unless otherwise stated, a group selected from fluorine, chlorine, bromine or iodine.

The term “aryl” includes phenyl and naphthyl. The term “heteroaryl” is intended to mean a 5-7 membered monocyclic aromatic or a fused 8-10 membered bicyclic aromatic ring containing 1 to 3 heteroatoms selected from oxygen, nitrogen and sulphur. Suitable examples of such monocyclic aromatic rings include thienyl, furyl, pyrrolyl, triazolyl, imidazolyl, oxazolyl, thiazolyl, oxadiazolyl, isothiazolyl, isoxazolyl, thiadiazolyl, pyrazolyl, pyrimidyl, pyridazinyl, pyrazinyl and pyridyl. Suitable examples of such fused aromatic rings include benzofused aromatic rings such as quinolinyl, isoquinolinyl, quinazolinyl, quinoxalinyl, cinnolinyl, naphthyridinyl, indolyl, indazolyl, pyrrolopyridinyl, benzofuranyl, benzothienyl, benzimidazolyl, benzoxazolyl, benzisoxazolyl, benzothiazolyl, benzisothiazolyl, benzoxadiazolyl, benzothiadiazolyl and the like. Heteroaryl groups, as described above, may be linked to the remainder of the molecule via a carbon atom or, when present, a suitable nitrogen atom except where otherwise indicated above. It will be appreciated that wherein the above mentioned aryl or heteroaryl groups have more than one substituent, said substituents may be linked to form a ring, for example a carboxyl and amine group may be linked to form an amide group.

The term “C₁₋₆ acyloxy” denotes an acyl radical attached to an oxygen atom wherein acyl has the same definition has described herein; some examples include but are not limited to acetyloxy, propionyloxy, butanoyloxy, iso-butanoyloxy, sec-butanoyloxy, t-butanoyloxy and the like.

The term “C₂₋₆ alkenyl” denotes a radical containing 2 to 6 carbons wherein at least one carbon-carbon double bond is present, some embodiments have 2 to 4 carbons, some embodiments have 2 to 3 carbons, and some embodiments have 2 carbons. Both E and Z isomers are embraced by the term “alkenyl.” Furthermore, the term “alkenyl” includes di- and tri-alkenyls. Accordingly, if more than one double bond is present, then the bonds may be all E or Z or a mixture of E and Z. Examples of an alkenyl include vinyl, allyl, 2-butenyl, 3-butenyl, 2-pentenyl, 3-pentenyl, 4-pentenyl, 2-hexenyl, 3-hexenyl, 4-hexenyl, 5-hexenyl, 2,4-hexadienyl and the like.

The term “C₁₋₆ alkoxy” as used herein denotes a radical alkyl, as defined herein, attached directly to an oxygen atom. Examples include methoxy, ethoxy, n-propoxy, iso-propoxy, n-butoxy, t-butoxy, iso-butoxy, sec-butoxy and the like.

The term “C₁₋₈ alkyl” denotes a straight or branched carbon radical containing 1 to 8 carbons, some embodiments have 1 to 6 carbons, some embodiments have 1 to 4 carbons, some embodiments have 1 to 3 carbons, and some embodiments have 1 or 2 carbons. Examples of an alkyl include, but are not limited to, methyl, ethyl, n-propyl, iso-propyl, n-butyl, sec-butyl, iso-butyl, t-butyl, pentyl, iso-pentyl, t-pentyl, neo-pentyl, 1-methylbutyl [i.e., —CH(CH₃)CH₂CH₂CH₃], 2-methylbutyl [i.e., —CH₂CH(CH₃)CH₂CH₃], n-hexyl and the like.

The term “C₁₋₆ alkylcarboxamido” or “C₁₋₆ alkylcarboxamide” denotes a single C₁₋₆ alkyl group attached to the nitrogen of an amide group, wherein alkyl has the same definition as found herein. The C₁₋₆ alkylcarboxamido may be represented by Formula II:

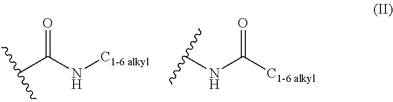

Examples include, but are not limited to, N-methylcarboxamide, N-ethylcarboxamide, N-n-propylcarboxamide, N-iso-propylcarboxamide, N-n-butylcarboxamide, N-sec-butylcarboxamide, N-iso-butylcarboxamide, N-t-butylcarboxamide and the like.

The term “C₁₋₃ alkylene” refers to a C₁₋₃ divalent straight carbon group. In some embodiments C₁₋₃ alkylene refers to, for example, —CH₂—, —CH₂CH₂—, —CH₂CH₂CH₂—, and the like. In some embodiments, C₁₋₃ alkylene refers to —CH—, —CHCH₂—, —CHCH₂CH₂—, and the like wherein these examples relate generally to the variable or claim element “Q”.

The term “C₁₋₆ alkylimino” denotes a C₁₋₆ alkyl radical attached directly to the carbon of the —C(═NH)— group wherein the definition of alkyl has the same definition as described herein; some examples include but are not limited to, 1-imino-ethyl [i.e., C(═NH)CH₃], 1-imino-propyl [i.e., —C(═NH)CH₂CH₃], 1-imino-2-methyl-propyl [i.e., C(═NH)CH(CH₃)₂], and the like.

The term “C₁₋₆ alkylsulfinyl” denotes a C₁₋₆ alkyl radical attached to a sulfoxide radical of the formula: —S(O)— wherein the alkyl radical has the same definition as described herein. Examples include, but are not limited to, methylsulfinyl, ethylsulfinyl, n-propylsulfinyl, iso-propylsulfinyl, n-butylsulfinyl, sec-butylsulfinyl, iso-butylsulfinyl, t-butylsulfinyl, and the like.

The term “C₁₋₆ alkylsulfonamide” refers to the groups of Formula III:

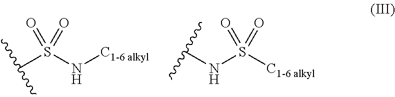

wherein C₁₋₆ alkyl has the same definition as described herein.

The term “C₁₋₆ alkylsulfonyl” denotes a C₁₋₆ alkyl radical attached to a sulfone radical of the formula: —S(O)₂— wherein the alkyl radical has the same definition as described herein. Examples include, but are not limited to, methylsulfonyl, ethylsulfonyl, n-propylsulfonyl, iso-propyl sulfonyl, n-butyl sulfonyl, sec-butyl sulfonyl, iso-butyl sulfonyl, t-butylsulfonyl, and the like.

The term “C₁₋₆ alkylthio” denotes a C₁₋₆ alkyl radical attached to a sulfide of the formula: —S— wherein the alkyl radical has the same definition as described herein. Examples include, but are not limited to, methylsulfanyl (i.e., CH₃S—), ethylsulfanyl, n-propylsulfanyl, iso-propylsulfanyl, n-butylsulfanyl, sec-butylsulfanyl, iso-butylsulfanyl, t-butylsulfanyl, and the like.

The term “C₁₋₆ alkylthiocarboxamide” denotes a thioamide of the following Formula IV:

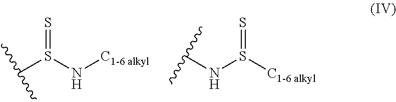

wherein C₁₋₄ alkyl has the same definition as described herein.

The term “C₁₋₆ alkylthioureyl” denotes the group of the formula: —NC(S)N— wherein one or both of the nitrogens are substituted with the same or different C1-6 alkyl groups and alkyl has the same definition as described herein. Examples of an alkylthioureyl include, but are not limited to, CH₃NHC(S)NH—, NH₂C(S)NCH₃—, (CH₃)₂N(S)NH—, (CH₃)₂N(S)NH—, (CH3)₂N(S)NCH₃—, CH₃CH₂NHC(S)NH—, CH₃CH₂NHC(S)NCH₃—, and the like.

The term “C₁₋₆ alkylureyl” denotes the group of the formula: —NC(O)N— wherein one or both of the nitrogens are substituted with the same or different C₁₋₆ alkyl group wherein alkyl has the same definition as described herein. Examples of an alkylureyl include, but are not limited to, CH₃NHC(O)NH—, NH₂C(O)NCH₃—, (CH₃)₂NC(O)NH—, (CH₃)₂NC(O)NH—, (CH₃)₂NC(O)NCH₃—, CH₃CH₂NHC(O)NH—, CH₃CH₂NHC(O)NCH₃—, and the like.

The term “C₂₋₆ alkynyl” denotes a radical containing 2 to 6 carbons and at least one carbon-carbon triple bond, some embodiments have 2 to 4 carbons, some embodiments have 2 to 3 carbons, and some embodiments have 2 carbons. Examples of an alkynyl include, but are not limited to, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like. The term “alkynyl” includes di- and triynes.

The term “amino” denotes the group —NH₂.

The term “C₁₋₆ alkylamino” denotes one alkyl radical attached to an amino radical wherein the alkyl radical has the same meaning as described herein. Some examples include, but are not limited to, methylamino, ethylamino, n-propylamino, iso-propylamino, n-butylamino, sec-butylamino, iso-butylamino, t-butylamino, and the like. Some embodiments are “C₁₋₂ alkylamino.”

The term “aryl” denotes an aromatic ring radical containing 6 to 10 ring carbons. Examples include phenyl and naphthyl.

The term “arylalkyl” defines a C₁-C₄ alkylene, such as —CH₂—, —CH₂CH₂— and the like, which is further substituted with an aryl group. Examples of an “arylalkyl” include benzyl, phenethylene and the like.

The term “arylcarboxamido” denotes a single aryl group attached to the nitrogen of an amide group, wherein aryl has the same definition as found herein. An example is N-phenylcarboxamide.

The term “arylureyl” denotes the group —NC(O)N— where one of the nitrogens are substituted with an aryl.

The term “benzyl” denotes the group —CH₂C₆H₅.

The term “carbo-C₁₋₆-alkoxy” refers to a C₁₋₆ alkyl ester of a carboxylic acid, wherein the alkyl group is as defined herein. Examples include, but are not limited to, carbomethoxy, carboethoxy, carbopropoxy, carboisopropoxy, carbobutoxy, carbo-sec-butoxy, carbo-iso-butoxy, carbo-t-butoxy, carbo-n-pentoxy, carbo-iso-pentoxy, carbo-t-pentoxy, carbo-neo-pentoxy, carbo-n-hexyloxy, and the like.

The term “carboxamide” refers to the group —CONH₂.

The term “carboxy” or “carboxyl” denotes the group —CO₂H; also referred to as a carboxylic acid group.

The term “cyano” denotes the group —CN.

The term “C₄₋₇ cycloalkenyl” denotes a non-aromatic ring radical containing 4 to 7 ring carbons and at least one double bond; some embodiments contain 4 to 6 carbons; some embodiments contain 4 to 5 carbons; some embodiments contain 4 carbons. Examples include cyclobutenyl, cyclopentenyl, cyclopentenyl, cyclohexenyl, and the like.

The term “C₃₋₇ cycloalkyl” denotes a saturated ring radical containing 3 to 7 carbons; some embodiments contain 3 to 6 carbons; some embodiments contain 3 to 5 carbons; some embodiments contain 5 to 7 carbons; some embodiments contain 3 to 4 carbons. Examples include cyclopropyl, cyclobutyl, cyclopentyl, cyclohexyl, cycloheptyl and the like.

The term “C₂₋₈ dialkylamino” denotes an amino substituted with two of the same or different C₁₋₄ alkyl radicals wherein alkyl radical has the same definition as described herein. Some examples include, but are not limited to, dimethylamino, methylethylamino, diethylamino, methylpropylamino, methylisopropylamino, ethylpropylamino, ethylisopropylamino, dipropylamino, propylisopropylamino and the like. Some embodiments are “C₂₋₄ dialkylamino.”

The term “C₂₋₈ dialkylcarboxamido” or “C₂₋₈ dialkylcarboxamide” denotes two alkyl radicals, that are the same or different, attached to an amide group, wherein alkyl has the same definition as described herein. A C₂₋₈ dialkylcarboxamido may be represented by Formula V:

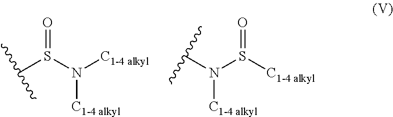

wherein C₁₋₄ has the same definition as described herein. Examples of a dialkylcarboxamide include, but are not limited to, N,N-dimethylcarboxamide, N-methyl-N-ethylcarboxamide, N,N-diethylcarboxamide, N-methyl-N-isopropylcarboxamide, and the like.

The term “C₂₋₈ dialkylsulfonamide” refers to one of the following groups shown in Formula VI:

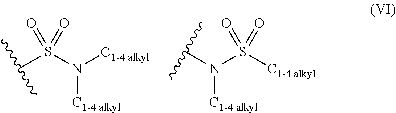

wherein C₁₋₄ has the same definition as described herein, for example but not limited to, methyl, ethyl, n-propyl, isopropyl, and the like.

The term “C₂₋₈ dialkylthiocarboxamido” or “C₂₋₈ dialkylthiocarbox-amide” denotes two alkyl radicals, that are the same or different, attached to a thioamide group, wherein alkyl has the same definition as described herein. A C₂₋₈dialkylthiocarboxamido or C₂₋₈ dialkylthiocarboxamide may be represented by the Formula VII:

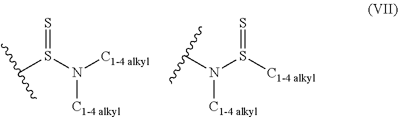

Examples of a dialkylthiocarboxamide include, but are not limited to, N,N-dimethylthiocarboxamide, N-methyl-N-ethylthiocarboxamide and the like.

The term “ethynylene” refers to the carbon-carbon triple bond group as represented Formula VIII:

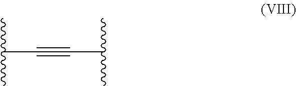 [SMILES: *C#C*]

The term “formyl” refers to the group —CHO.

The term “C₁₋₆ haloalkoxy” denotes a haloalkyl, as defined herein, which is directly attached to an oxygen atom. Examples include, but are not limited to, difluoromethoxy, trifluoromethoxy, 2,2,2-trifluoroethoxy, pentafluoroethoxy and the like.

The term “C₁₋₆ haloalkyl” denotes an C₁₋₆ alkyl group, defined herein, wherein the alkyl is substituted with one halogen up to fully substituted and a fully substituted C₁₋₆ haloalkyl can be represented by the formula CₙL₂ₙ₊₁ wherein L is a halogen and “n” is 1, 2, 3 or 4. When more than one halogen is present, they may be the same or different and selected from the group consisting of F, Cl, Br and I, preferably F. Examples of C₁₋₄ haloalkyl groups include, but are not limited to, fluoromethyl, difluoromethyl, trifluoromethyl, chlorodifluoromethyl, 2,2,2-trifluoroethyl, pentafluoroethyl and the like.

The term “C₁₋₆ haloalkylcarboxamide” denotes an alkylcarboxamide group, defined herein, wherein the alkyl is substituted with one halogen up to fully substituted represented by the formula CₙL₂ₙ₊₁ wherein L is a halogen and “n” is 1, 2, 3 or 4. When more than one halogen is present, they may be the same or different and selected from the group consisting of F, Cl, Br and I, preferably F.

The term “C₁₋₆ haloalkylsulfinyl” denotes a haloalkyl radical attached to a sulfoxide group of the formula: —S(O)— wherein the haloalkyl radical has the same definition as described herein. Examples include, but are not limited to, trifluoromethylsulfinyl, 2,2,2-trifluoroethylsulfinyl, 2,2-difluoroethylsulfinyl and the like.

The term “C₁₋₆ haloalkylsulfonyl” denotes a haloalkyl radical attached to a sulfone group of the formula: —S(O)₂— wherein haloalkyl has the same definition as described herein. Examples include, but are not limited to, trifluoromethylsulfonyl, 2,2,2-trifluoroethyl sulfonyl, 2,2-difluoroethylsulfonyl and the like.

The term “C₁₋₆ haloalkylthio” denotes a haloalkyl radical directly attached to a sulfur wherein the haloalkyl has the same meaning as described herein. Examples include, but are not limited to, trifluoromethylthio (i.e., CF₃S—, also referred to as trifluoromethylsulfanyl), 1,1-difluoroethylthio, 2,2,2-trifluoroethylthio and the like.

The term “halogen” or “halo” denotes a fluoro, chloro, bromo or iodo group.

The term “heteroaryl” denotes an aromatic ring system that may be a single ring, two fused rings or three fused rings wherein at least one ring carbon is replaced with a heteroatom selected from, but are not limited to, the group consisting of O, S and N wherein the N can be optionally substituted with H, C₁₋₄ acyl or C₁₋₄ alkyl. Examples of heteroaryl groups include, but are not limited to, pyridyl, benzofuranyl, pyrazinyl, pyridazinyl, pyrimidinyl, triazinyl, quinoline, benzoxazole, benzothiazole, 1H-benzimidazole, isoquinoline, quinazoline, quinoxaline and the like. In some embodiments, the heteroaryl atom is O, S, NH. Examples include, but are not limited to, pyrrole, indole, and the like. Other examples include, but are not limited to, those in Table 1, Table 2, and the like.

The term “heterocyclic” denotes a non-aromatic carbon ring (i.e., C₃₋₇ cycloalkyl or C₄₋₇ cycloalkenyl as defined herein) wherein one, two or three ring carbons are replaced by a heteroatom selected from, but are not limited to, the group consisting of O, S, N, wherein the N can be optionally substituted with H, C₁₋₄ acyl or C₁₋₄ alkyl, and ring carbon atoms optionally substituted with oxo or a thiooxo thus forming a carbonyl or thiocarbonyl group. The heterocyclic group is a 3-, 4-, 5-, 6- or 7-membered containing ring. Examples of a heterocyclic group include, but are not limited to, aziridin-1-yl, aziridin-2-yl, azetidin-1-yl, azetidin-2-yl, azetidin-3-yl, piperidin-1-yl, piperidin-4-yl, morpholin-4-yl, piperazin-1-yl, piperazin-4-yl, pyrrolidin-1-yl, pyrrolidin-3-yl, [1,3]-dioxolan-2-yl and the like.

The term “heterocycliccarboxamido” denotes a heterocyclic group, as defined herein, with a ring nitrogen where the ring nitrogen is bonded directly to the carbonyl forming an amide. Examples include those in Formula IX, but are not limited to,

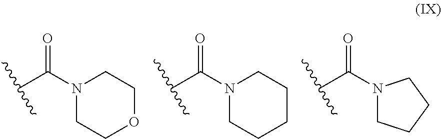

and the like.

The term “heterocyclicsulfonyl” denotes a heterocyclic group, as defined herein, with a ring nitrogen where the ring nitrogen is bonded directly to an —SO₂-group forming an sulfonamide. Examples include those in Formula X, but are not limited to,

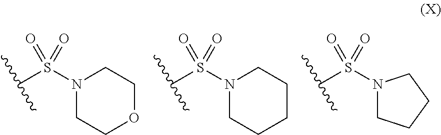

and the like.

The term “hydroxyl” refers to the group —OH.

The term “hydroxylamino” refers to the group —NHOH.

The term “nitro” refers to the group —NO₂.

The term “C₄₋₇ oxo-cycloalkyl” refers to a C₄₋₇ cycloalkyl, as defined herein, wherein one of the ring carbons is replaced with a carbonyl. Examples of C₄₋₇ oxo-cycloalkyl include, but are not limited to, 2-oxo-cyclobutyl, 3-oxo-cyclobutyl, 3-oxo-cyclopentyl, 4-oxo-cyclohexyl, and the like and represented by the structures respectively in Formula XI:

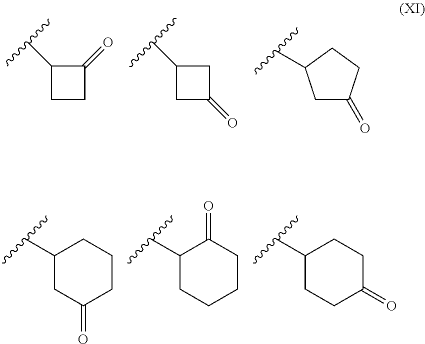

The term “perfluoroalkyl” denotes the group of the formula —CₙF₂ₙ₊₁; stated differently, a perfluoroalkyl is an alkyl as defined herein wherein the alkyl is fully substituted with fluorine atoms and is therefore considered a subset of haloalkyl. Examples of perfluoroalkyls include CF₃, CF₂CF₃, CF₂CF₂CF₃, CF(CF₃)₂, CF₂CF₂CF₂CF₃, CF₂CF(CF₃)₂, CF(CF₃)CF₂CF₃ and the like.

The term “phenoxy” refers to the group C₆H₅O—.

The term “phenyl” refers to the group C₆H₅—.

The term “sulfonic acid” refers to the group —SO₃H.

The term “thiol” denotes the group —SH.

Pharmaceutically acceptable salts, hydrates, polymorphs, or solutes of molecular compounds are well known in the art, and it will be appreciated that in some embodiments, any compound or agent described herein can exist as a pharmaceutically acceptable salt, hydrate, polymorph, or solute as appropriate. In some embodiments, the compounds described herein can form acid addition salts thereof. It will be appreciated that for use in medicine the salts of the compounds described herein should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. The present disclosure includes within its scope all possible stoichiometric and non-stoichiometric forms. In some embodiments, the compounds described herein may be prepared in crystalline or non-crystalline form, and, if crystalline, may optionally be solvated, e.g. as the hydrate. This disclosure includes within its scope stoichiometric solvates (e.g. hydrates) as well as compounds containing variable amounts of solvent (e.g. water). Certain compounds described herein are capable of existing as polymorphisms or in stereoisomeric forms (e.g. diastereomers and enantiomers) and the disclosure extends to each of these polymorphs, stereoisomeric forms, and to mixtures thereof including racemates. The different stereoisomeric forms may be separated one from the other by the usual methods, or any given isomer may be obtained by stereospecific or asymmetric synthesis. The disclosure also extends to any tautomeric forms and mixtures thereof.

Before the present compositions and methods are described, it is to be understood that this disclosure is not limited to the particular processes, compositions, or methodologies described, as these may vary. Moreover, the processes, compositions, and methodologies described in particular embodiments are interchangeable. Therefore, for example, a composition, dosages regimen, route of administration, and so on described in a particular embodiment may be used in any of the methods described in other particular embodiments. It is also to be understood that the terminology used in the description is for the purpose of describing the particular versions or embodiments only, and is not intended to limit the scope of the present disclosure which will be limited only by the appended claims. Unless defined otherwise, all technical and scientific terms used herein have the same meanings as commonly understood by one of ordinary skill in the art. Although any methods similar or equivalent to those described herein can be used in the practice or testing of embodiments of the present disclosure, the preferred methods are now described. All publications and references mentioned herein are incorporated by reference. Nothing herein is to be construed as an admission that the disclosure is not entitled to antedate such disclosure by virtue of prior disclosure.

Although any methods and materials similar or equivalent to those described herein can be used in the practice or testing of embodiments of the present disclosure, the preferred methods, devices, and materials are now described.

Some embodiments, are directed to compositions and methods of treating neurodegenerative diseases, reducing side effects including side effects associated with cholinesterase inhibitors, in a subject being treated for a neurodegenerative disease, enhancing the efficacy of the treatment of a neurodegenerative disease, enhancing the standard of care of the treatment of a neurodegenerative disease or a combination thereof. In some embodiments, the compositions described and the compositions to be used in the methods described herein may comprise a 5-HT₆ receptor antagonist, a 5-HT_{2A} inverse agonist, an NMDA receptor antagonist or a combination thereof in combination with an acetylcholinesterase inhibitor and an anti-cholinergic agent.

In some embodiments, the 5-HT₆ receptor antagonist is a compound of formula (XII):

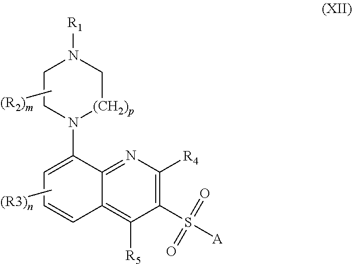

wherein: R₁ and R₂ independently represent hydrogen or C₁₋₆ alkyl or R₁ is linked to R₂ to form a group (CH₂)₂, (CH₂)₃ or (CH₂)₄; R₃, R₄ and R₅ independently represent hydrogen, halogen, cyano, —CF₃, —CF₃O, C₁₋₆ alkyl, C₁₋₆ alkoxy, C₁₋₆ alkanoyl or a group —CONR₆R₇; R₆ and R₇ independently represent hydrogen or C₁₋₆ alkyl or together may be fused to form a 5- to 7-membered aromatic or non-aromatic heterocyclic ring optionally interrupted by an O or S atom; m represents an integer from 1 to 4, such that when m is an integer greater than 1, two R₂ groups may instead be linked to form a group CH₂, (CH₂)₂ or (CH₂)₃; n represents an integer from 1 to 3; p represents 1 or 2; A represents a group —Ar₁ or —Ar₂Ar₃; Ar₁, Ar₂ and Ar₃ independently represent an aryl group or a heteroaryl group, both of which may be optionally substituted by one or more (e.g., 1, 2 or 3) substituents which may be the same or different, and which are selected from the group consisting of halogen, hydroxy, cyano, nitro, trifluoromethyl, trifluoromethoxy, C₁₋₆ alkyl, trifluoromethanesulfonyloxy, pentafluoroethyl, C₁₋₆ alkoxy, arylC₁₋₆ alkoxy, C₁₋₆ alkylthio, C₁₋₆ alkoxyC₁₋₆ alkyl, C₃₋₇cycloalkylC₁₋₆ alkoxy, C₁₋₆ alkanoyl, C₁₋₆ alkoxycarbonyl, C₁₋₆ alkylsulfonyl, C₁₋₆ alkylsulfinyl, C₁₋₆ alkylsulfonyloxy, C₁₋₆alkylsulfonyl C₁₋₆ alkyl, arylsulfonyl, arylsulfonyloxy, arylsulfonyl C₁₋₆ alkyl, C₁₋₆ alkylsulfonamido, C₁₋₆ alkylamido, C₁₋₆ alkylsulfonamido C₁₋₆ alkyl, C₁₋₆ alkylamidoC₁₋₆ alkyl, arylsulfonamido, arylcarboxamido, arylsulfonamido C₁₋₆ alkyl, arylcarboxamido C₁₋₆ alkyl, aroyl, aroylC₁₋₆ alkyl, arylC₁₋₆ alkanoyl, or a group CONR₈R₉ or SO₂NR₈R₉, wherein R₈ and R₉ independently represent hydrogen or C₁₋₆ alkyl or together may be fused to form a 5- to 7-membered aromatic or non-aromatic heterocyclic ring optionally interrupted by an O or S atom; or pharmaceutically acceptable salts, hydrates or solvates thereof.

Alkyl groups, whether alone or as part of another group, may be straight chain or branched and the group's alkoxy and alkanoyl shall be interpreted similarly. Alkyl moieties are more preferably C₁₋₄ alkyl, e.g., methyl or ethyl. The term ‘halogen’ is used herein to describe, unless otherwise stated, a group selected from fluorine, chlorine, bromine or iodine.

The term “aryl” includes phenyl and naphthyl. The term “heteroaryl” is intended to mean a 5-7 membered monocyclic aromatic or a fused 8-10 membered bicyclic aromatic ring containing 1 to 3 heteroatoms selected from oxygen, nitrogen and sulphur. Suitable examples of such monocyclic aromatic rings include thienyl, furyl, pyrrolyl, triazolyl, imidazolyl, oxazolyl, thiazolyl, oxadiazolyl, isothiazolyl, isoxazolyl, thiadiazolyl, pyrazolyl, pyrimidyl, pyridazinyl, pyrazinyl and pyridyl. Suitable examples of such fused aromatic rings include benzofused aromatic rings such as quinolinyl, isoquinolinyl, quinazolinyl, quinoxalinyl, cinnolinyl, naphthyridinyl, indolyl, indazolyl, pyrrolopyridinyl, benzofuranyl, benzothienyl, benzimidazolyl, benzoxazolyl, benzisoxazolyl, benzothiazolyl, benzisothiazolyl, benzoxadiazolyl, benzothiadiazolyl and the like. Heteroaryl groups, as described above, may be linked to the remainder of the molecule via a carbon atom or, when present, a suitable nitrogen atom except where otherwise indicated above. It will be appreciated that wherein the above mentioned aryl or heteroaryl groups have more than one substituent, said substituents may be linked to form a ring, for example a carboxyl and amine group may be linked to form an amide group.

In some embodiments, R₁ represents hydrogen, methyl, ethyl, isopropyl, isobutyl or 2,2-dimethylpropyl. In some embodiments, R₁ represents hydrogen or methyl, especially hydrogen. Preferably R₂ represents hydrogen, methyl (e.g., 3-methyl, 2-methyl, 3,3-dimethyl or 2,5-dimethyl) or is linked to R₁ to form a (CH₂)₃ group. More preferably, R₂ represents hydrogen or methyl (e.g., 3-methyl), especially hydrogen.

In some embodiments, R₃ represents hydrogen, methyl (e.g., 6-methyl) or halogen (e.g., 7-chloro). More preferably, R₃ represents hydrogen.

In some embodiments, R₄ and R₅ independently represent hydrogen or methyl, especially hydrogen.

In some embodiments, n represents 1. In some embodiments, m and p independently represent 1 or 2, more preferably m and p both represent 1. In some embodiments, m represents 2 and both R2 groups are linked to form a CH₂ group linking C-2 and C-5 of the piperazine ring.

In some embodiments, when A represents a group —Ar₁, Ar₁ preferably represents optionally substituted phenyl or pyridyl, or in some embodiments, a phenyl optionally substituted with halogen (e.g., chlorine, fluorine or bromine), cyano, trifluoromethyl or trifluoromethoxy. In some embodiments, Ar₁ is unsubstituted phenyl or phenyl substituted by halogen (e.g., 2-chloro, 3-chloro, 4-chloro, 2-fluoro, 3-fluoro, 4-fluoro or 4-bromo), C₁₋₆ alkyl (e.g., 2-methyl or 4-methyl), C1-6 alkoxy (e.g., 2-methoxy), trifluoromethyl (e.g., 2-trifluoromethyl or 3-trifluoromethyl) or trifluoromethoxy (e.g., 2-trifluoromethoxy).

In some embodiments, when A represents a group —Ar₂—Ar₃, Ar₂ and Ar₃ both independently represent phenyl or monocyclic heteroaryl group as defined above. In some embodiments, A represents a group —Ar₁. In some embodiments, —Ar₁ is unsubstituted phenyl.

The compounds of formula (XII) can form acid addition salts thereof. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids, e.g., hydrochloric, hydrobromic, sulfuric, nitric or phosphoric acid; and organic acids, e.g., succinic, maleic, acetic, fumaric, citric, tartaric, benzoic, p-toluenesulfonic, methanesulfonic or naphthalenesulfonic acid. The present disclosure includes within its scope all possible stoichiometric and non-stoichiometric forms.

The compounds of formula (XII) may be prepared in crystalline or non-crystalline form, and, if crystalline, may optionally be solvated, e.g., as the hydrate. This disclosure includes within its scope stoichiometric solvates (e.g., hydrates) as well as compounds containing variable amounts of solvent (e.g., water). Certain compounds of formula (I) are capable of existing in stereoisomeric forms (e.g., diastereomers and enantiomers) and the disclosure extends to each of these stereoisomeric forms and to mixtures thereof including racemates. The different stereoisomeric forms may be separated one from the other by the usual methods, or any given isomer may be obtained by stereospecific or asymmetric synthesis. The disclosure also extends to any tautomeric forms and mixtures thereof.

In some embodiments, the 5-HT₆ receptor antagonist is 3-phenylsulfonyl-8-piperazinyl-1yl-quinoline (Formula XIII)

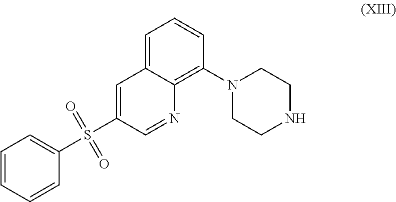 [SMILES: O=S(=O)(c1ccccc1)c1cnc2c(N3CCNCC3)cccc2c1]

or pharmaceutically acceptable salts, hydrates or solvates thereof; a therapeutically effective amount of at least one additional therapeutic agent useful for the treatment of a neurodegenerative disease; and at least one pharmaceutically acceptable excipient; wherein the composition is suitable for oral administration. 3-phenylsulfonyl-8-piperazinyl-1yl-quinoline is also known as intepirdine, RVT-101, and SB-742457.

In some embodiments, the 5-HT_{2A} inverse agonist is a compound of Formula XIV:

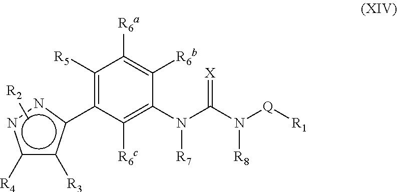

or a pharmaceutically acceptable salt, hydrate or solvate thereof; wherein:
i) R₁ is aryl or heteroaryl each optionally substituted with R₉, R₁₀, R₁₁, R₁₂, R₁₃, R₁₄, and R₁₅ each selected independently from the group consisting of C₁₋₆ acyl, C₁₋₆ acyloxy, C₂₋₆ alkenyl, C₁₋₆alkoxy, C₁₋₆ alkyl, C₁₋₆ alkylcarboxamide, C₂₋₆ alkynyl, C₁₋₆alkylsulfonamide, C₁₋₆ alkylsulfinyl, C₁₋₆ alkylsulfonyl, C₁₋₆ alkylureyl, amino, C₁₋₆ alkylamino, C₂₋₈dialkylamino, C₁₋₆ alkylimino, carbo-C₁₋₆-alkoxy, carboxamide, carboxy, cyano, C₃₋₇ cycloalkyl, C₂₋₈ dialkylcarboxamide, C₂₋₈dialkylsulfonamide, halogen, C₁₋₆ haloalkoxy, C₁₋₆ haloalkyl, C₁₋₆haloalkylsulfinyl, C₁₋₆ haloalkylsulfonyl, C₁₋₆ haloalkylthio, heterocyclic, hydroxyl, thiol, nitro, phenoxy and phenyl, or two adjacent R₉, R₁₀, R₁₁, R₁₂, R₁₃, R₁₄, and R₁₅ together with the atoms to which they are attached form a C₅₋₇ cycloalkyl group or heterocyclic group each optionally substituted with F, Cl, or Br; and wherein said C₂₋₆ alkenyl, C₁₋₆ alkyl, C₂₋₆ alkynyl, C₁₋₆ alkylamino, C₁₋₆ alkylimino, C₂₋₈ dialkylamino, heterocyclic, and phenyl are each optionally substituted with 1 to 5 substituents selected independently from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆alkynyl, C₁₋₆ alkylsulfonamide, C₁₋₆ alkylsulfinyl, C₁₋₆alkylsulfonyl, C₁₋₆ alkylthio, C₁₋₆ alkylureyl, amino, C₁₋₆alkylamino, C₂₋₈ dialkylamino, carbo-C₁₋₆-alkoxy, carboxamide, carboxy, cyano, C₃₋₇ cycloalkyl, C₂₋₈ dialkylcarboxamide, halogen, C₁₋₆ haloalkoxy, C₁₋₆ haloalkyl, C₁₋₆ haloalkylsulfinyl, C₁₋₆haloalkylsulfonyl, C₁₋₆ haloalkylthio, hydroxyl, thiol and nitro;
ii) R₂ is selected from the group consisting of H, C₁₋₆ alkyl, C₂₋₆alkenyl, C₂₋₆ alkynyl and C₃₋₇ cycloalkyl;
iii) R₃ is selected from the group consisting of H, C₂₋₆ alkenyl, C₁₋₆ alkylcarboxamide, C₂₋₆ alkynyl, C₁₋₆ alkylsulfonamide, carbo-C₁₋₆-alkoxy, carboxamide, carboxy, cyano, C₃₋₇ cycloalkyl, C₂₋₈ dialkylcarboxamide, halogen, heteroaryl and phenyl; and wherein each of said C₂₋₆ alkenyl, C₁₋₆ alkyl, C₂₋₆ alkynyl, C₁₋₆alkylsulfonamide, C₃₋₇ cycloalkyl, heteroaryl and phenyl groups can be optionally substituted with 1 to 5 substituents selected independently from the group consisting of C₁₋₅ acyl, C₁₋₅ acyloxy, C₂₋₆ alkenyl, C₁₋₄ alkoxy, C₁₋₈ alkyl, C₁₋₆ alkylamino, C₂₋₈dialkylamino, C₁₋₄ alkylcarboxamide, C₂₋₆ alkynyl, sulfonamide, C₁₋₄ alkylsulfinyl, C₁₋₄ alkylsulfonyl, C₁₋₄ alkylureyl, amino, carbo-C₁₋₆-alkoxy, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄haloalkylsulfonyl, C₁₋₄ haloalkylthio, hydroxyl, nitro and sulfonamide;
iv) R₄ is selected from the group consisting of H, C₁₋₆ acyl, C₁₋₆acyloxy, C₂₋₆ alkenyl, C₁₋₆ alkoxy, C₁₋₆ alkyl, C₁₋₆alkylcarboxamide, C₂₋₆ alkynyl, C₁₋₆ alkylsulfonamide, C₁₋₆alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈dialkylcarboxamide, C₂₋₈ dialkylsulfonamide, halogen, C₁₋₆haloalkoxy, C₁₋₆ haloalkyl, C₁₋₆ haloalkylsulfinyl, C₁₋₆haloalkylsulfonyl, C₁₋₆ haloalkylthio, hydroxyl, thiol, nitro and sulfonamide;
v) R₅ is selected from the group consisting of C₁₋₆ acyl, C₁₋₆acyloxy, C₂₋₆ alkenyl, C₁₋₆ alkoxy, C₁₋₆ alkyl, C₁₋₆alkylcarboxamide, C₂₋₆ alkynyl, C₁₋₆ alkylsulfonamide, C₁₋₆alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈dialkylcarboxamide, 8 dialkylsulfonamide, halogen, C₁₋₆haloalkoxy, C₁₋₆ haloalkyl, C₁₋₆ haloalkylsulfinyl, C₁₋₆haloalkylsulfonyl, C₁₋₆ haloalkylthio, hydroxyl, thiol, nitro and sulfonamide, wherein said C₁₋₆ alkoxy group can be optionally substituted with 1 to 5 substituents selected independently from the group consisting of C₁₋₅ acyl, C₁₋₅ acyloxy, C₂₋₆ alkenyl, C₁₋₄alkoxy, C₁₋₈ alkyl, amino, C₁₋₆ alkylamino, C₂₋₈ dialkylamino, C₁₋₄alkylcarboxamide, C₂₋₆ alkynyl, C₁₋₄ alkylsulfonamide, C₁₋₄alkylsulfinyl, C₁₋₄ alkylsulfonyl, C₁₋₄ alkylthio, C₁₋₄ alkylureyl, amino, carb-C₁₋₆-alkoxy, carboxamide, carboxy, cyano, C₃₋₆cycloalkyl, C₂₋₆ dialkylcarboxamide, halogen, C₁₋₄ haloalkoxy, C₁₋₄haloalkyl, C₁₋₄ haloalkylsulfinyl, C₁₋₄ haloalkylsulfonyl, C₁₋₄haloalkylthio, hydroxyl, nitro and phenyl; and wherein said amino and phenyl are each optionally substituted with 1 to 5 further substituents selected from the group consisting of halogen and carbo-C₁₋₆-alkoxy;
vi) R₆ₐ, R_{6b}, and R_{6c} are each independently selected from the group consisting of H, C₁₋₆ acyl, C₁₋₆ acyloxy, C₂₋₆ alkenyl, C₁₋₆alkoxy, C₁₋₆ alkyl, C₁₋₆ alkylcarboxamide, C₂₋₆ alkynyl, C₁₋₆alkylsulfonamide, C₁₋₆ alkylsulfinyl, C₁₋₆ alkylsulfonyl, C₁₋₆alkylthio, C₁₋₆ alkylureyl, amino, C₁₋₆ alkylamino, C₂₋₈dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro and sulfonamide;
vii) R₇ and R₈ are independently H or C₁₋₈ alkyl;
viii) X is O or S; and
ix) Q is C₁₋₃ alkylene optionally substituted with 1 to 4 substituents selected from the group consisting of C₁₋₃ alkyl, C₁₋₄ alkoxy, carboxy, cyano, C₁₋₃ haloalkyl, halogen and oxo; or Q is a bond.

In some embodiments, the 5-HT_{2A} inverse agonist is selected from nelotanserin, pimavanserin, pruvanserin, eplivanserin, volinanserin, glemanserin, ketanserin, ritanserin, clozapine, or a pharmaceutically acceptable salt, hydrate, polymorph, or solvate thereof. In some embodiments, the 5-HT_{2A} inverse agonist is 1-[3-(4-bromo-2-methyl-2H-pyrazol-3-yl)-4-methoxy-phenyl]-3-(2,4-difluoro-phenyl)-urea (also known as nelotanserin, RVT-102, and APD-125) or a pharmaceutically acceptable salt, hydrate, polymorph, or solvate thereof.

Some embodiments of the present disclosure encompass certain diaryl and arylheteroaryl urea derivatives as shown in the following Formula XV

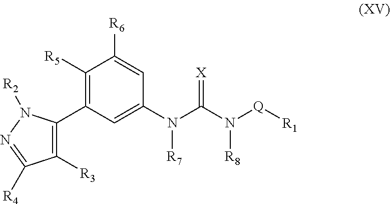

wherein:
i) R₁ is aryl or heteroaryl optionally substituted with R₉, R₁₀, R₁₁, R₁₂, R₁₃, R₁₄, and R₁₅ selected independently from the group consisting of C₁₋₆ acyl, C₁₋₆ acyloxy, C₂₋₆ alkenyl, C₁₋₆ alkoxy, C₁₋₆ alkyl, C₁₋₆alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇cycloalkyl, C₂₋₈ dialkylcarboxamide, C₂₋₈ dialkylsulfonamide, halogen, C₁₋₆ haloalkoxy, C₁₋₆ haloalkyl, C₁₋₆haloalkylsulfinyl, C₁₋₆ haloalkylsulfonyl, C₁₋₆ haloalkylthio, hydroxyl, thiol, nitro, phenoxy and phenyl, or two adjacent R₉, R₁₀, R₁₁, R₁₂, R₁₃, R₁₄, and R₁₅ together with the atoms to which they are attached form a C₅₋₇cycloalkyl group or heterocyclic group each optionally substituted with F, Cl, or Br; and wherein each of said C₂₋₆ alkenyl, C₁₋₆ alkyl, C₂₋₆ alkynyl and phenyl groups can be optionally substituted with 1 to 5 substituents selected independently from the group consisting of C₁₋₆ acyl, C₁₋₆ acyloxy, C₂₋₆ alkenyl, C₁₋₆ alkoxy, C₁₋₆alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, halogen, C₁₋₆ haloalkoxy, C₁₋₆ haloalkyl, C₁₋₆haloalkylsulfinyl, C₁₋₆ haloalkylsulfonyl, C₁₋₆ haloalkylthio, hydroxyl, thiol and nitro;
ii) R₂ is selected from the group consisting of C₁₋₆ alkyl, C₂₋₆ alkenyl, C₂₋₆ alkynyl and C₃₋₇ cycloalkyl;
iii) R₃ is selected from the group consisting of H, C₂₋₆ alkenyl, C₁₋₆ alkyl, C₁₋₆ alkylcarboxamide, C₂₋₆ alkynyl, C₁₋₆ alkylsulfonamide, carbo-C₁₋₆-alkoxy, carboxamide, carboxy, cyano, C₃₋₇ cycloalkyl, C₂₋₈dialkylcarboxamide, halogen, heteroaryl and phenyl; and wherein each of said C₂₋₆ alkenyl, C₁₋₆ alkyl, C₂₋₆alkynyl, C₁₋₆ alkylsulfonamide, C₃₋₇ cycloalkyl, heteroaryl and phenyl groups can be optionally substituted with 1 to 5 substituents selected independently from the group consisting of C₁₋₅ acyl, C₁₋₅ acyloxy, C₂₋₆ alkenyl, C₁₋₄ alkoxy, C₁₋₈ alkyl, C₁₋₆ alkylamino, C₂₋₈ dialkylamino, C₁₋₄ alkylcarboxamide, C₂₋₆ alkynyl, sulfonamide, C₁₋₄ alkylsulfinyl, C₁₋₄ alkylsulfonyl, C₁₋₄ alkylthio, C₁₋₄ alkylureyl, amino, carbo-C₁₋₆-alkoxy, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, nitro and sulfonamide;
iv) R₄ is selected from the group consisting of H, C₁₋₆ acyl, C₁₋₆ acyloxy, C₂₋₆ alkenyl, C₁₋₆ alkoxy, C₁₋₆alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆haloalkyl, C₁₋₆ haloalkylsulfinyl, C₁₋₆ haloalkylsulfonyl, C₁₋₆ haloalkylthio, hydroxyl, thiol, nitro and sulfonamide;
v) R₅ is selected from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₄ alkylsulfonyl, C₁₋₆alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆haloalkyl, C₁₋₆ haloalkylsulfinyl, C₁₋₆ haloalkylsulfonyl, C₁₋₆ haloalkylthio, hydroxyl, thiol, nitro and sulfonamide, wherein said C₁₋₆ alkoxy group can be optionally substituted with 1 to 5 substituents selected independently from the group consisting of C₁₋₅ acyl, C₁₋₅ acyloxy, C₂₋₆ alkenyl, C₁₋₄ alkoxy, C₁₋₈ alkyl, C₁₋₆alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C₁₋₄ alkylthio, C₁₋₄ alkylureyl, amino, carbo-C₁₋₆-alkoxy, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄haloalkylsulfonyl, C₁₋₄ haloalkylthio, hydroxyl, nitro and phenyl, and wherein said phenyl is optionally substituted with 1 to 5 halogen atoms;
vi) R₆ is selected from the group consisting of H, C₁₋₆ acyl, C₁₋₆ acyloxy, C₂₋₆ alkenyl, C₁₋₆ alkoxy, C₁₋₆alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆haloalkyl, C₁₋₆ haloalkylsulfinyl, C₁₋₆ haloalkylsulfonyl, C₁₋₆ haloalkylthio, hydroxyl, thiol, nitro and sulfonamide;
vii) R₇ and R₈ are independently H or C₁₋₈ alkyl;
viii) X is O or S; and
ix) Q is C₁₋₃ alkylene optionally substituted with 1 to 4 substituents selected from the group consisting of C₁₋₃alkyl, C₁₋₄ alkoxy, carboxy, cyano, C₁₋₃ haloalkyl, halogen and oxo; or Q is a bond; or a pharmaceutically acceptable salt, hydrate or solvate thereof.

It is appreciated that certain features of the disclosure, which are, for clarity, described in the context of separate embodiments, may also be provided in combination in a single embodiment. Conversely, various features of the disclosure which are, for brevity, described in the context of a single embodiment, may also be provided separately or in any suitable subcombination.

As used herein, “substituted” indicates that at least one hydrogen atom of the chemical group is replaced by a non-hydrogen substituent or group, the non-hydrogen substituent or group can be monovalent or divalent. When the substituent or group is divalent, then it is understood that this group is further substituted with another substituent or group. When a chemical group herein is “substituted” it may have up to the full valance of substitution; for example, a methyl group can be substituted by 1, 2, or 3 substituents, a methylene group can be substituted by 1 or 2 substituents, a phenyl group can be substituted by 1, 2, 3, 4, or 5 substituents, a naphthyl group can be substituted by 1, 2, 3, 4, 5, 6, or 7 substituents and the like. Likewise, “substituted with one or more substituents” refers to the substitution of a group with one substituent up to the total number of substituents physically allowed by the group. Further, when a group is substituted with more than one group they can be identical or they can be different.

Compounds of the disclosure can also include tautomeric forms, such as keto-enol tautomers, and the like. Tautomeric forms can be in equilibrium or sterically locked into one form by appropriate substitution. It is understood that the various tautomeric forms are within the scope of the compounds of the present disclosure.

Compounds of the disclosure can also include all isotopes of atoms occurring in the intermediates and/or final compounds. Isotopes include those atoms having the same atomic number but different mass numbers. For example, isotopes of hydrogen include deuterium and tritium.

It is understood and appreciated that compounds of the present disclosure may have one or more chiral centers, and therefore can exist as enantiomers and/or diastereomers. The disclosure is understood to extend to and embrace all such enantiomers, diastereomers and mixtures thereof, including but not limited to racemates. Accordingly, some embodiments of the present disclosure pertain to compounds of the present disclosure that are R enantiomers. Further, some embodiments of the present disclosure pertain to compounds of the present disclosure that are S enantiomers. In examples where more than one chiral center is present, some embodiments of the present disclosure include compounds that are RS or SR enantiomers. In further embodiments, compounds of the present disclosure are RR or SS enantiomers. It is understood that compounds of the present disclosure are intended to represent all individual enantiomers and mixtures thereof, unless stated or shown otherwise.

In some embodiments, R1 is aryl or heteroaryl each optionally substituted with R9, R10, R11, R12, R13, R14, and R15 each selected independently from the group consisting of C1-6 acyl, C1-6 acyloxy, C2-6 alkenyl, C1-6 alkoxy, C1-6 alkyl, C1-6alkylcarboxamide, C2-6 alkynyl, C1-6 alkylsulfonamide, C1-6 alkylsulfinyl, C1-6 alkylsulfonyl, C1-6 alkylthio, C1-6 alkylureyl, amino, C1-6 alkylamino, C2-8 dialkylamino, C1-6 alkylimino, carbo-C1-6-alkoxy, carboxamide, carboxy, cyano, C3-7cycloalkyl, C2-8 dialkylcarboxamide, C2-8 dialkylsulfonamide, halogen, C1-6 haloalkoxy, C1-6 haloalkyl, C1-6 haloalkylsulfinyl, C1-6 haloalkylsulfonyl, C1-6 haloalkylthio, heterocyclic, hydroxyl, thiol, nitro, phenoxy and phenyl, wherein said C2-6 alkenyl, C1-6 alkyl, C2-6 alkynyl, C1-6 alkylamino, C1-6 alkylimino, C2-8 dialkylamino, heterocyclic, and phenyl are each optionally substituted with 1 to 5 substituents selected independently from the group consisting of C1-6 acyl, C1-6 acyloxy, C2-6alkenyl, C1-6 alkoxy, C1-6 alkyl, C1-6 alkylcarboxamide, C2-6 alkynyl, C1-6 alkylsulfonamide, C1-6 alkylsulfinyl, C1-6alkylsulfonyl, C1-6 alkylthio, C1-6 alkylureyl, amino, C1-6 alkylamino, C2-8 dialkylamino, carbo-C1-6-alkoxy, carboxamide, carboxy, cyano, C3-7 cycloalkyl, C2-8 dialkylcarboxamide, halogen, C1-6 haloalkoxy, C1-6 haloalkyl, C1-6 haloalkylsulfinyl, C1-6 haloalkylsulfonyl, C1-6 haloalkylthio, hydroxyl, thiol and nitro;

Some embodiments of the present disclosure pertain to compounds wherein R1 is phenyl or naphthyl each optionally substituted with R9, R10, R11, R12, R13, R14, and R15 each selected independently from the group consisting of C1-6 acyl, C1-6 alkoxy, C1-6 alkyl, C1-6 alkylsulfonyl, amino, C1-6 alkylamino, C2-8 dialkylamino, C1-6 alkylimino, carbo-C1-6-alkoxy, carboxamide, carboxy, cyano, C3-7 cycloalkyl, halogen, C1-6 haloalkoxy, C1-6 haloalkyl, heterocyclic, hydroxyl, nitro, and phenyl, or two adjacent R9, R10, R11, R12, R13, R14, and R15 together with the atoms to which they are attached form a C5-7 cycloalkyl group or heterocyclic group each optionally substituted with F; and wherein said C1-6 alkyl, C1-6 alkylimino, and heterocyclic are each optionally substituted with 1 to 5 substituents selected independently from the group consisting of C1-6 acyl, C1-6 alkoxy, C1-6 alkyl, C1-6 alkylsulfonyl, amino, C1-6 alkylamino, C2-8 dialkylamino, carboxamide, cyano, C3-7cycloalkyl, halogen, C1-6 haloalkoxy, C1-6 haloalkyl, and hydroxyl.

Some embodiments of the present disclosure pertain to compounds wherein R1 is phenyl optionally substituted with R9, R10, R11, R12, and R13 each selected independently from the group consisting of C1-6 acyl, C1-6 alkoxy, C1-6 alkyl, C1-6alkylsulfonyl, amino, C1-6 alkylamino, C2-8 dialkylamino, C1-6 alkylimino, carbo-C1-6-alkoxy, carboxamide, carboxy, cyano, C3-7 cycloalkyl, halogen, C1-6 haloalkoxy, C1-6 haloalkyl, heterocyclic, hydroxyl, nitro, and phenyl, or two adjacent R9, R10, R11, R12, and R13 together with the atoms to which they are attached form a C5-7 cycloalkyl group or heterocyclic group each optionally substituted with F; and wherein said C1-6 alkyl, C1-6 alkylimino, and heterocyclic are each optionally substituted with 1 to 5 substituents selected independently from the group consisting of C1-6 acyl, C1-6 alkoxy, C1-6 alkyl, C1-6 alkylsulfonyl, amino, C1-6 alkylamino, C2-8 dialkylamino, carboxamide, cyano, C3-7 cycloalkyl, halogen, C1-6haloalkoxy, C1-6 haloalkyl, and hydroxyl.

Some embodiments of the present disclosure pertain to compounds wherein R1 is phenyl or naphthyl each optionally substituted with R9, R10, R11, R12, R13, R14, and R15 each selected independently from the group consisting of C1-6 acyl, C1-6 alkoxy, C1-6 alkyl, amino, C1-6 alkylamino, C2-8 dialkylamino, C1-6 alkylimino, cyano, halogen, C1-6 haloalkoxy, C1-6haloalkyl, heterocyclic, hydroxyl, nitro, and phenyl, or two adjacent R9, R10, R11, R12, R13, R14, and R15 together with the atoms to which they are attached form a C5-7 cycloalkyl group or heterocyclic group each optionally substituted with F; and wherein said C1-6 alkyl, C1-6 alkylimino, and heterocyclic are each optionally substituted with 1 to 5 substituents selected independently from the group consisting of C1-6 alkyl, amino, C1-6 alkylamino, C2-8 dialkylamino, and hydroxyl.

Some embodiments of the present disclosure pertain to compounds wherein R1 is phenyl optionally substituted with R9, R10, R11, R12, and R13 each selected independently from the group consisting of C1-6 acyl, C1-6 alkoxy, C1-6 alkyl, amino, C1-6alkylamino, C2-8 dialkylamino, C1-6 alkylimino, cyano, halogen, C1-6 haloalkoxy, C1-6 haloalkyl, heterocyclic, hydroxyl, nitro, and phenyl, or two adjacent R9, R10, R11, R12, and R13 together with the atoms to which they are attached form a C5-7cycloalkyl group or heterocyclic group each optionally substituted with F; and wherein said C1-6 alkyl, C1-6 alkylimino, and heterocyclic are each optionally substituted with 1 to 5 substituents selected independently from the group consisting of C1-6 alkyl, amino, C1-6 alkylamino, C2-8 dialkylamino, and hydroxyl.

Some embodiments of the present disclosure pertain to compounds wherein R1 is phenyl or naphthyl optionally substituted with R9, R10, R11, R12, R13, R14, and R15 each selected independently from the group consisting of —C(O)CH3, —OCH3, —CH3, —CH(CH3)2, —CH(OH)CH3, —N(CH3)2, (2-dimethylamino-ethyl)-methyl-amino [i.e., —N(CH3)CH2CH2N(CH3)2], (3-dimethylamino-propyl)-methyl-amino [i.e., —N(CH3)CH2CH2CH2N(CH3)2], —C(═NOH)CH3, cyano, —F, —Cl, —Br, —OCF3, —CF3, 4-methyl-piperazin-1-yl, morpholin-4-yl, 4-methyl-piperidin-1-yl, hydroxyl, nitro, and phenyl.

Some embodiments of the present disclosure pertain to compounds wherein R1 is phenyl optionally substituted with R9, R10, R11, R12, and R13, R14 each selected independently from the group consisting of —C(O)CH3, —OCH3, —CH3, —CH(CH3)2, —CH(OH)CH3, —N(CH3)2, (2-dimethylamino-ethyl)-methyl-amino [i.e., —N(CH3)CH2CH2N(CH3)2], (3-dimethylamino-propyl)-methyl-amino [i.e., —N(CH3)CH2CH2CH2N(CH3)2], —C(═NOH)CH3, cyano, —F, —Cl, —Br, —OCF3, —CF3, 4-methyl-piperazin-1-yl, morpholin-4-yl, 4-methyl-piperidin-1-yl, hydroxyl, nitro, and phenyl.

Some embodiments of the present disclosure pertain to compounds wherein R1 is phenyl or naphthyl optionally substituted with R9, R10, R11, R12, R13, R14, and R15 each selected independently from the group consisting of —OCH3, —CH3, cyano, —F, —Cl, —Br, —OCF3, and —CF3.

Some embodiments of the present disclosure pertain to compounds wherein R1 is phenyl optionally substituted with R9, R10, R11, R12, and R13 each selected independently from the group consisting of —OCH3, —CH3, cyano, —F, —Cl, —Br, —OCF3, and —CF3.

Some embodiments of the present disclosure pertain to compounds wherein R1 is phenyl and can be represented by the Formula XVI shown below:

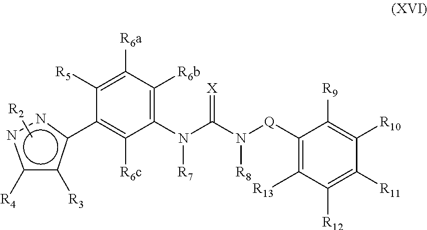

wherein each variable in the above formula has the same meaning as described herein, supra and infra. In some embodiments, R₇ and R₈ are both —H, Q is a bond, and X is O.

Some embodiments of the present disclosure pertain to compounds wherein R₁ is phenyl and can be represented by Formula XVII as shown below:

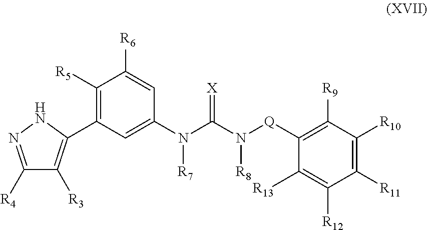

wherein R₉ to R₁₃ substituents are each selected independently from the group consisting of H, C₁₋₆ acyl, C₁₋₆ acyloxy, C₁₋₆ alkoxy, C₁₋₆ alkyl, C₁₋₆ alkylcarboxamide, C₁₋₆ alkylsulfonamide, C₁₋₆ alkylsulfinyl, C₁₋₆ alkylsulfonyl, C₁₋₆ alkylthio, amino, C₁₋₆alkylamino, C₂₋₈ dialkylamino, carbo-C₁₋₆-alkoxy, carboxamide, carboxy, cyano, halogen, C₁₋₆ haloalkoxy, C₁₋₆ haloalkyl, hydroxyl, nitro and phenyl, or two adjacent substituents together with the phenyl form a C₅₋₇ cycloalkyl optionally comprising 1 to 2 oxygen atoms; and wherein each said C₁₋₆ alkyl and phenyl groups can be optionally substituted with 1 to 5 substituents selected independently from the group consisting of C₁₋₆ alkoxy, C₁₋₆ alkyl, amino, cyano, halogen, C₁₋₆haloalkoxy, C₁₋₆ haloalkyl, hydroxyl and nitro.

In some embodiments, R1 is phenyl optionally substituted with R9 to R13 substituents selected independently from the group consisting of C1-6 acyl, C1-6 alkoxy, C1-6 alkyl, cyano, halogen, C1-6 haloalkoxy, C1-6 haloalkyl, nitro and phenyl; and wherein said phenyl can be optionally substituted with 1 to 5 substituents selected independently from the group consisting of C1-6 alkoxy, C1-6 alkyl, cyano, halogen, C1-6 haloalkoxy, C1-6 haloalkyl and nitro.

In some embodiments, R1 is phenyl optionally substituted with R9 to R13 substituents selected independently from the group consisting of C1-6 acyl, C1-6 alkoxy, C1-6 alkyl, cyano, halogen, C1-6 haloalkoxy, C1-6 haloalkyl, nitro and phenyl.

In some embodiments, R1 is phenyl optionally substituted with R9 to R13 substituents selected independently from the group consisting of —C(O)CH3, —C(O)CH2CH3, —C(O)CH(CH3)2, —C(O)CH2CH2CH3, —C(O)CH2CH(CH3)2, —OCH3, —OCH2CH3, —OCH(CH3)2, —OCH2CH2CH3, —OCH2CH(CH3)2, —CH3, —CH2CH3, —CH(CH3)2, —CH2CH2CH3, —CH2CH(CH3)2, —CH2CH2CH2CH3, cyano, F, Cl, Br, I, —OCF3, —OCHF2, —OCFH2, —OCF2CF3, —OCH2CF3, —CF3, —CHF2, —CFH2, —CF2CF3, —CH2CF3, nitro and phenyl.

In some embodiments, R1 is phenyl optionally substituted with R9 to R13 substituents are each selected independently from the group consisting of —C(O)CH3, —OCH3, —CH3, —CH(CH3)2, —CH(OH)CH3, —N(CH3)2, (2-dimethylamino-ethyl)-methyl-amino, (3-dimethylamino-propyl)-methyl-amino, —C(═NOH)CH3, cyano, —F, —Cl, —Br, —OCF3, —CF3, 4-methyl-piperazin-1-yl, morpholin-4-yl, 4-methyl-piperidin-1-yl, hydroxyl, nitro, and phenyl.

In some embodiments, R1 is phenyl optionally substituted with R9, R10, R11, R12 and R13 substituents selected independently from the group consisting of —C(O)CH3, —OCH3, —CH3, cyano, —F, —Cl, —Br, —OCF3, —CF3, nitro and phenyl.

Some embodiments of the present disclosure pertain to compounds wherein R1 is naphthyl optionally substituted with R9 R10R11 R12 R13 R14 and R15 substituents selected independently from the group consisting of C1-6 acyl, C1-6 acyloxy, C1-6alkoxy, C1-6 alkyl, C1-6 alkylcarboxamide, C1-6 alkylsulfonamide, C1-6 alkylsulfinyl, C1-6 alkylsulfonyl, C1-6 alkylthio, amino, C1-6 alkylamino, C2-8 dialkylamino, carbo-C1-6-alkoxy, carboxamide, carboxy, cyano, halogen, C1-6 haloalkoxy, C1-6haloalkyl, hydroxyl and nitro; and wherein said C1-6 alkyl can be optionally substituted with 1 to 5 substituents selected independently from the group consisting of C1-6 alkoxy, C1-6 alkyl, amino, cyano, halogen, C1-6 haloalkoxy, C1-6 haloalkyl, hydroxyl and nitro.

In some embodiments, R1 is naphthyl optionally substituted with R9, R10, R11, R12, R13, R14 and R15 substituents selected independently from the group consisting of C1-6 acyl, C1-6 alkoxy, C1-6 alkyl, cyano, halogen, C1-6 haloalkoxy, C1-6haloalkyl and nitro.

In some embodiments, R1 is naphthyl optionally substituted with R9, R10, R11, R12, R13, R14 and R15 substituents selected independently from the group consisting of C(O)CH3, —C(O)CH2CH3, —C(O)CH(CH3)2, —C(O)CH2CH2CH3, —C(O)CH2CH(CH3)2, —OCH3, —OCH2CH3, —OCH(CH3)2, —OCH2CH2CH3, —OCH2CH(CH3)2, —CH3, —CH2CH3, —CH(CH3)2, —CH2CH2CH3, —CH2CH(CH3)2, —CH2CH2CH2CH3, cyano, —F, —Cl, —Br, —I, —OCF3, —OCHF2, —OCFH2, —OCF2CF3, —OCHF2CF3, —CF3, —CHF2, —CFH2, —CF2CF3, —CH2CF3 and nitro.

In some embodiments, R1 is naphthyl optionally substituted with R9, R10, R11, R12, R13, R14 and R15 substituents selected independently from the group consisting of —C(O)CH3, —C(O)CH2CH3, —C(O)CH(CH3)2, —C(O)CH2CH2CH3, —C(O)CH2CH(CH3)2, —OCH3, —OCH2CH3, —OCH(CH3)2, —OCH2CH2CH3, —OCH2CH(CH3)2, —CH3, —CH2CH3, —CH(CH3)2, —CH2CH2CH3, —CH2CH(CH3)2, —CH2CH2CH2CH3, cyano, —F, —Cl, —Br, —I, —OCF3, —OCHF2, —OCFH2, —OCF2CF3, —OCH2CF3, —CF3, —CHF2, —CFH2, —CF2CF3, —CH2CF3 and nitro.

In some embodiments, R1 is naphthyl optionally substituted with R9, R10, R11, R12, R13, R14 and R15 substituents selected independently from the group consisting of —C(O)CH3, —OCH3, —CH3, cyano, —F, —Cl, —Br, —OCF3, —CF3 and nitro.

Some embodiments of the present disclosure pertain to compounds wherein R1 is heteroaryl optionally substituted with R9, R10, R11, R12, and R13 each selected independently from the group consisting of C1-6 acyl, C1-6 alkoxy, C1-6alkyl, amino, C1-6 alkylamino, C2-8 dialkylamino, C1-6 alkylimino, cyano, halogen, C1-6 haloalkoxy, C1-6 haloalkyl, heterocyclic, hydroxyl, nitro, and phenyl, or two adjacent R9, R10, R11, R12, R13, R14, and R15 together with the atoms to which they are attached form a C5-7 cycloalkyl group or heterocyclic group each optionally substituted with F; and wherein said C1-6 alkyl, C1-6alkylimino, and heterocyclic are each optionally substituted with 1 to 5 substituents selected independently from the group consisting of C1-6 alkyl, amino, C1-6 alkylamino, C2-8 dialkylamino, and hydroxyl.

Some embodiments of the present disclosure pertain to compounds wherein R1 is heteroaryl optionally substituted with R9, R10, R11, R12, and R13 each selected independently from the group consisting of —C(O)CH3, —OCH3, —CH3, —CH(CH3)2, —CH(OH)CH3, —N(CH3)2, (2-dimethylamino-ethyl)-methyl-amino, (3-dimethylamino-propyl)-methyl-amino, —C(═NOH)CH3, cyano, —F, —Cl, —Br, —OCF3, —CF3, 4-methyl-piperazin-1-yl, morpholin-4-yl, 4-methyl-piperidin-1-yl, hydroxyl, nitro, and phenyl.

Some embodiments of the present disclosure pertain to compounds wherein R1 is heteroaryl optionally substituted with R9, R10, R11, R12, and R13 each selected independently from the group consisting of —OCH3, —CH3, cyano, —F, —Cl, —Br, —OCF3, and —CF3.

Some embodiments of the present disclosure pertain to compounds wherein R1 is heteroaryl optionally substituted with R9, R10, R11, R12, and R13 each selected independently from the group consisting of C1-6 acyl, C1-6 acyloxy, C1-6 alkoxy, C1-6alkyl, C1-6 alkylcarboxamide, C1-6 alkylsulfonamide, C1-6 alkylsulfinyl, C1-6 alkylsulfonyl, C1-6 alkylthio, amino, C1-6alkylamino, C2-8 dialkylamino, carbo-C1-6-alkoxy, carboxamide, carboxy, cyano, halogen, C1-6 haloalkoxy, C1-6 haloalkyl, hydroxyl, nitro and phenyl, or two adjacent R9, R10, R11, R12, R13, R14, and R15 together with the atoms to which they are attached form a C5-7 cycloalkyl group or heterocyclic group; and wherein each of said C1-6 alkyl and phenyl groups can be optionally substituted with 1 to 5 substituents selected independently from the group consisting of C1-6 alkoxy, C1-6 alkyl, amino, cyano, halogen, C1-6 haloalkoxy, C1-6 haloalkyl, hydroxyl and nitro.

In some embodiments, R1 is heteroaryl optionally substituted with R9, R10, R11, R12 and R13 each selected independently from the group consisting of C1-6 acyl, C1-6 alkoxy, C1-6 alkyl, cyano, halogen, C1-6 haloalkoxy, C1-6 haloalkyl, nitro and phenyl; and wherein said phenyl can be optionally substituted with 1 to 5 substituents selected independently from the group consisting of C1-6 alkoxy, C1-6 alkyl, cyano, halogen, C1-6 haloalkoxy, C1-6 haloalkyl and nitro.

In some embodiments, R1 is heteroaryl optionally substituted with R9, R10, R11, R12 and R13 each selected independently from the group consisting of C1-6 acyl, C1-6 alkoxy, C1-6 alkyl, cyano, halogen, C1-6 haloalkoxy, C1-6 haloalkyl, nitro and phenyl.

In some embodiments, R1 is heteroaryl optionally substituted with R9, R10, R11, R12, and R13 each selected independently from the group consisting of —C(O)CH3, —C(O)CH2CH3, —C(O)CH(CH3)2, —C(O)CH2CH2CH3, —C(O)CH2CH(CH3)2, —OCH3, —OCH2CH3, —OCH(CH3)2, —OCH2CH2CH3, —OCH2CH(CH3)2, —CH3, —CH2CH3, —CH(CH3)2, —CH2CH2CH3, —CH2CH(CH3)2, —CH2CH2CH2CH3, cyano, —F, —Cl, —Br, —I, —OCF3, —OCHF2, —OCFH2, —OCF2CF3, —OCH2CF3, —CF3, —CHF2, —CFH2, —CF2CF3, —CH2CF3, nitro and phenyl.

In some embodiments, R1 is heteroaryl optionally substituted with R9, R10, R11, R12, and R13 each selected independently from the group consisting of —C(O)CH3, —OCH3, —CH3, cyano, —F, —Cl, —Br, —OCF3, —CF3, nitro and phenyl. In some embodiments, R1 is heteroaryl optionally substituted with R9, R10, R11, R12, and R13 selected independently from the group consisting of H, —C(O)CH3, —OCH3, —CH3, cyano, —F, —Cl, —Br, —OCF3, —CF3, nitro and phenyl.

In some embodiments, R1 is heteroaryl having 5-atoms in the aromatic ring, examples of which are represented by the following formulae in Table 1:

TABLE 1

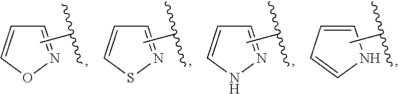

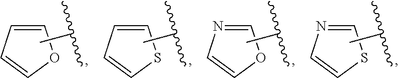

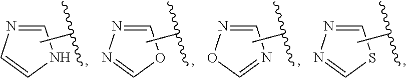

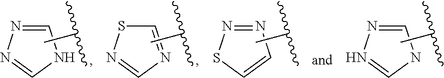

wherein the 5-membered heteroaryl is bonded at any available position of the ring, for example, a imidazolyl ring can be bonded at one of the ring nitrogens (i.e., imidazol-1-yl group) or at one of the ring carbons (i.e., imidazol-2-yl, imidazol-4-yl or imidazol-5-yl group).

In some embodiments, R₁ is a 6-membered heteroaryl, for example, a 6-membered heteroaryl as shown in Table 2:

TABLE 2

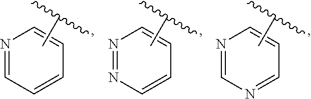

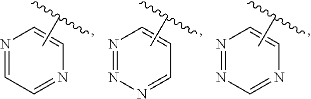

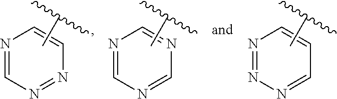

wherein the heteroaryl group is bonded at any ring carbon. In some embodiments, R₁ is selected from the group consisting of pyridinyl, pyridazinyl, pyrimidinyl and pyrazinyl. In some embodiments, R₁ is pyridinyl.

In some embodiments R1 is a heteroaryl, for example but is not limited to those shown in Tables 1 and 2, optionally substituted with 1 to 3 substituents selected from the group consisting of C1-6 acyl, C1-6 acyloxy, C2-6 alkenyl, C1-6 alkoxy, C1-6 alkyl, C1-6 alkylcarboxamide, C2-6 alkynyl, C1-6 alkylsulfonamide, C1-6 alkylsulfinyl, C1-6 alkylsulfonyl, C1-6 alkylthio, C1-6alkylureyl, amino, C1-6 alkylamino, C2-8 dialkylamino, carbo-C1-6-alkoxy, carboxamide, carboxy, cyano, C3-7 cycloalkyl, C2-8 dialkylcarboxamide, C2-8 dialkylsulfonamide, halogen, C1-6 haloalkoxy, C1-6 haloalkyl, C1-6 haloalkylsulfinyl, C1-6haloalkylsulfonyl, C1-6 haloalkylthio, hydroxyl, thiol, nitro, phenoxy and phenyl; and wherein each of said C2-6 alkenyl, C1-6alkyl, C2-6 alkynyl and phenyl groups can be optionally substituted with 1 to 5 substituents selected independently from the group consisting of C1-6 acyl, C1-6 acyloxy, C2-6 alkenyl, C1-6 alkoxy, C1-6 alkyl, C1-6 alkylcarboxamide, C2-6 alkynyl, C1-6alkylsulfonamide, C1-6 alkylsulfinyl, C1-6 alkylsulfonyl, C1-6 alkylthio, C1-6 alkylureyl, amino, C1-6 alkylamino, C2-8dialkylamino, carbo-C1-6-alkoxy, carboxamide, carboxy, cyano, C3-7 cycloalkyl, C2-8 dialkylcarboxamide, halogen, C1-6haloalkoxy, C1-6 haloalkyl, C1-6 haloalkylsulfinyl, C1-6 haloalkylsulfonyl, C1-6 haloalkylthio, hydroxyl, thiol and nitro.

Some embodiments of the present disclosure pertain to compounds wherein R2 is H or C1-6 alkyl.

Some embodiments of the present disclosure pertain to compounds wherein R2 is C1-6 alkyl. In some embodiments, R2 is selected from the group consisting of —CH3, —CH2CH3, —CH(CH3)2, —CH2CH2CH3, —CH2CH(CH3)2 and —CH2CH2CH2CH3. In some embodiments, R2 is —CH3 or —CH(CH3)2.

It is understood that when R2 is H, then tautomers are possible. It is well understood and appreciated in the art that pyrazoles can exist in various tautomeric forms. Two possible tautomeric forms are illustrated below as Formula XVIId and XVIId′:

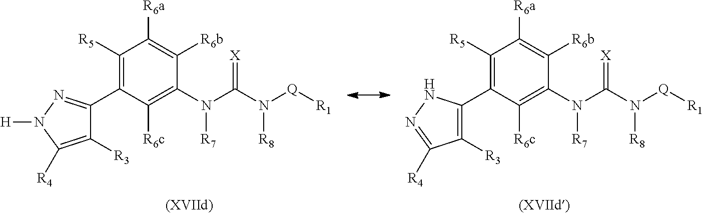

It is further understood that tautomeric forms can also have corresponding nomenclature for each represented tautomer, for example, Formula XVIId and Formula XVIId′ can be represented by the general chemical names 1H-pyrazol-3-yl and 2H-pyrazole-3-yl respectively. Therefore, the present disclosure includes all tautomers and the various nomenclature designations.

One aspect of the present disclosure pertains to certain compounds as shown in Formula XVIII:

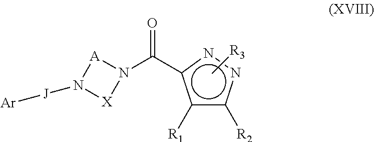

or a pharmaceutically acceptable salt, hydrate or solvate thereof; wherein R₁, R₂, R₃, Ar, A, X and J have the same definitions as described herein, supra and infra.

In some embodiments, the compounds of the present disclosure are other than 1-(4-(1H-pyrazole-3-carbonyl)piperazin-1-yl)-2-(4-fluoro-1H-indol-3-yl)ethane-1,2-dione, represented by the Formula XIX below:

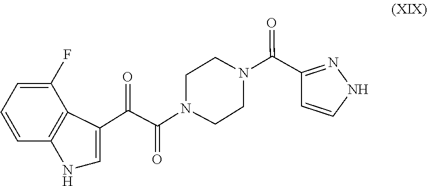 [SMILES: O=C(C(=O)N1CCN(C(=O)c2cc[nH]n2)CC1)c1c[nH]c2cccc(F)c12]

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables (e.g., R1, R2, R3, Ar, A, X and J) contained within the generic chemical formulae described herein for example, are specifically embraced by the present disclosure just as if they were explicitly disclosed, to the extent that such combinations embrace compounds that result in stable compounds (i.e., compounds that can be isolated, characterized and tested for biological activity). In addition, all subcombinations of the chemical groups listed in the embodiments describing such variables, as well as all subcombinations of uses and medical indications described herein, are also specifically embraced by the present disclosure just as if each of such subcombination of chemical groups and subcombination of uses and medical indications were explicitly disclosed herein.

It is understood and appreciated that compounds of Formula 2a and formulae related therefrom may have one or more chiral centers, and therefore can exist as enantiomers and/or diastereomers. The disclosure is understood to extend to and embrace all such enantiomers, diastereomers and mixtures thereof, including but not limited to racemates. It is understood that compounds of Formula XIX and formulae used throughout this disclosure are intended to represent all individual enantiomers and mixtures thereof, unless stated or shown otherwise.

Some embodiments of the present disclosure pertain to compounds of Formula XX:

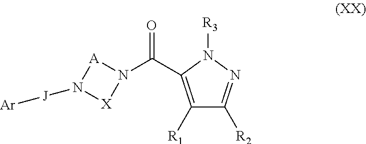

Some embodiments of the present disclosure pertain to compounds of Formula XXI:

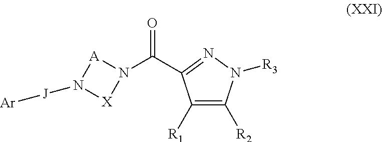

In some embodiments, each R1 and R2 is selected independently from the group consisting of H, C1-C6 alkyl, C1-C6 alkylaryl, aryl, C3-C7 cycloalkyl, C1-C6 haloalkyl, halogen, heteroaryl, and nitro.

In some embodiments, R1 and R2 is selected independently from the group consisting of H, methyl, ethyl, isopropyl, t-butyl, 2-methylphenyl, phenyl, cyclopropyl, trifluoromethyl, fluoro, chloro, bromo, iodo, furan-2-yl and nitro.

In some embodiments, R1 is H, halogen or C1-C6 alkylaryl; and R2 is H, C1-C6 alkyl, aryl, C3-C7 cycloalkyl, C1-C6 haloalkyl, heteroaryl or nitro.

In some embodiments, R1 is H, fluoro, chloro, bromo, iodo or 2-methylphenyl and R2 is H, methyl, ethyl, isopropyl, t-butyl, phenyl, cyclopropyl, trifluoromethyl, furan-2-yl or nitro.

In some embodiments, R1 and R2 together with the carbon atoms to which they are bonded form a C3-C7 carbocyclyl.

In some embodiments, R1 and R2 together with the carbon atoms to which they are bonded form a C5 carbocyclyl.

In some embodiments, R3 is selected from the group consisting of H, C1-C6 alkyl and aryl; and wherein aryl is optionally substituted with C1-C6 alkoxy.

In some embodiments, R3 is selected from the group consisting of H, C1-C6 alkyl and aryl; and wherein aryl is optionally substituted with methoxy.

In some embodiments, R3 is selected from the group consisting of H, methyl, ethyl, t-butyl, phenyl and 4-methoxyphenyl.

In some embodiments, A and X are each —CH2CH2-, each optionally substituted with C1-C3 alkyl.

In some embodiments, A and X are each —CH2CH2-, each optionally substituted with methyl.

In some embodiments, A and X are each independently —CH2CH2- or CH(CH3)CH2-.

In some embodiments, J is —CH2CH2-optionally substituted with 1, 2, 3 or 4 substituents selected independently from the group consisting of C1-C3 alkyl, hydroxyl, oxo and ═NO—C1-C3 alkyl.

In some embodiments, J is —CH2CH2-optionally substituted with 1, 2, 3 or 4 substituents selected independently from the group consisting of methyl, hydroxyl, oxo and ═NOCH3.

In some embodiments, J is —CH2CH2-, —C(═NOCH3)CH2-, —C═OCH2-, —CH(CH3)CH2-, —C(CH3)2CH2-, or —CHOHCH2-.

In some embodiments, Ar is aryl or heteroaryl each optionally substituted with 1, 2, 3, 4 or 5 substituents selected independently from the group consisting of C1-C6 alkoxy, C1-C6 alkylsulfonyl, C1-C6 haloalkoxy, C1-C6 haloalkyl, halogen and heterocyclyl.

In some embodiments, Ar is aryl or heteroaryl each optionally substituted with 1, 2, 3, 4 or 5 substituents selected independently from the group consisting of methoxy, methanesulfonyl, trifluoromethoxy, trifluoromethyl, fluoro, chloro and pyrrolidin-1-yl.

In some embodiments, Ar is naphthyl, 2-methoxyphenyl, 4-methoxyphenyl, 4-methanesulfonylphenyl, 4-trifluoromethoxyphenyl, 4-trifluoromethylphenyl, 2-fluorophenyl, 3-fluorophenyl, 4-fluorophenyl, 2,4-difluorophenyl, 3,4-difluorophenyl, 2-chlorophenyl, 3-chlorophenyl, 4-chlorophenyl and 6-chloro-1,3-dihydro-indol-2-one.

Some embodiments of the present disclosure pertain to compounds of Formula XXII:

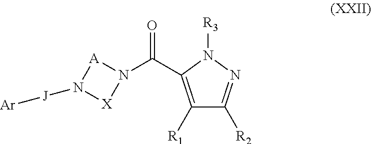

or a pharmaceutically acceptable salt, solvate or hydrate thereof; wherein:
R₁ is H, halogen or C₁-C₆alkylaryl;
R₂ is H, C₁-C₆ alkyl, aryl, C₃-C₇ cycloalkyl, C₁-C₆ haloalkyl, heteroaryl, or nitro; or
R₁ and R₂ together with the carbon atoms to which they are bonded form a C₃-C₇ carbocyclyl;
R₃ is H, C₁-C₆ alkyl, aryl, or aryl substituted with C₁-C₆ alkoxy;
A and X are each —CH₂CH₂—, each optionally substituted with C₁-C₃ alkyl;
J is —CH₂CH₂-optionally substituted with 1, 2, 3 or 4 substituents selected independently from the group consisting of C₁-C₃ alkyl, hydroxyl, oxo and ═NO—C₁-C₃ alkyl; and
Ar is aryl or heteroaryl each optionally substituted with 1, 2, 3, 4 or 5 substituents selected independently from the group consisting of C₁-C₆ alkoxy, C₁-C₆ alkylsulfonyl, C₁-C₆ haloalkoxy, C₁-C₆ haloalkyl, halogen and heterocyclyl.

Some embodiments of the present disclosure pertain to compounds of Formula XXIII:

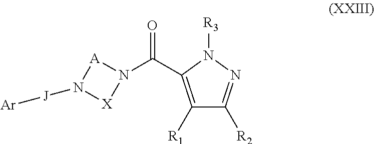

or a pharmaceutically acceptable salt, solvate or hydrate thereof; wherein:
R₁ is H, fluoro, chloro, bromo, iodo or 2-methylphenyl;
R₂ is H, methyl, ethyl, isopropyl, t-butyl, phenyl, cyclopropyl, trifluoromethyl, furan-2-yl or nitro; or
R₁ and R₂ together with the carbon atoms to which they are bonded form a C₅ carbocyclyl;
R₃ is H, methyl, ethyl, t-butyl, phenyl or 4-methoxyphenyl;
A and X are each independently —CH₂CH₂— or —CH(CH₃)CH₂—;
J is —CH₂CH₂—, —C(═NOMe)CH₂—, —C═OCH₂—, —CH(CH₃)CH₂—, —C(CH₃)₂CH₂—, or —CHOHCH₂—; and
Ar is naphthyl, 2-methoxyphenyl, 4-methoxyphenyl, 4-methanesulfonylphenyl, 4-trifluoromethoxyphenyl, 4-trifluoromethylphenyl, 2-fluorophenyl, 3-fluorophenyl, 4-fluorophenyl, 2,4-difluorophenyl, 3,4-difluorophenyl, 2-chlorophenyl, 3-chlorophenyl, 4-chlorophenyl and 6-chloro-1,3-dihydro-indol-2-one.

Some embodiments of the present disclosure pertain to compounds of Formula XXIV:

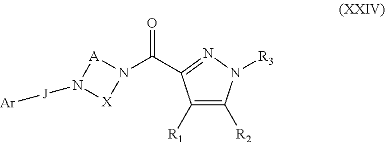

or a pharmaceutically acceptable salt, solvate or hydrate thereof; wherein:
R₁ is H, halogen or C₁-C₆ alkylaryl;
R₂ is H, C₁-C₆ alkyl, aryl, C₃-C₇ cycloalkyl, C₁-C₆ haloalkyl, heteroaryl, or nitro; or
R₁ and R₂ together with the carbon atoms to which they are bonded form a C₃-C₇ carbocyclyl;
R₃ is H, C₁-C₆ alkyl, aryl, or aryl substituted with C₁-C₆ alkoxy;
A and X are each —CH₂CH₂—, each optionally substituted with C₁-C₃ alkyl;
J is —CH₂CH₂-optionally substituted with 1, 2, 3 or 4 substituents selected independently from the group consisting of C₁-C₃ alkyl, hydroxyl, oxo and ═NO—C₁-C₃ alkyl; and
Ar is aryl or heteroaryl each optionally substituted with 1, 2, 3, 4 or 5 substituents selected independently from the group consisting of C₁-C₆ alkoxy, C₁-C₆ alkylsulfonyl, C₁-C₆haloalkoxy, C₁-C₆ haloalkyl, halogen and heterocyclyl.

Some embodiments of the present disclosure pertain to compounds of Formula XXV:

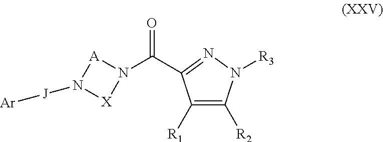

or a pharmaceutically acceptable salt, solvate or hydrate thereof; wherein:
R₁ is H, fluoro, chloro, bromo, iodo or 2-methylphenyl;
R₂ is H, methyl, ethyl, isopropyl, t-butyl, phenyl, cyclopropyl, trifluoromethyl, furan-2-yl or nitro; or
R₁ and R₂ together with the carbon atoms to which they are bonded form a C₅ carbocyclyl;
R₃ is H, methyl, ethyl, t-butyl, phenyl or 4-methoxyphenyl;
A and X are each independently —CH₂CH₂— or —CH(CH₃)CH₂—;
J is —CH₂CH₂—, —C(═NOMe)CH₂—, —C═OCH₂—, —CH(CH₃)CH₂—, —C(CH₃)₂CH₂—, or —CHOHCH₂—; and
Ar is naphthyl, 2-methoxyphenyl, 4-methoxyphenyl, 4-methanesulfonylphenyl, 4-trifluoromethoxyphenyl, 4-trifluoromethylphenyl, 2-fluorophenyl, 3-fluorophenyl, 4-fluorophenyl, 2,4-difluorophenyl, 3,4-difluorophenyl, 2-chlorophenyl, 3-chlorophenyl, 4-chlorophenyl and 6-chloro-1,3-dihydro-indol-2-one.

In some embodiments, where R₁, R₂ and R₃ are all H; and A and X are both CH₂CH₂—; and J is (CO)₂; then Ar is a moiety other than heteroaryl substituted with halogen.

As used herein, an “anti-cholinergic agent” is an agent which blocks the stimulation of acetylcholine receptors. In some embodiments, an anti-cholinergic agent is a an agent which blocks the stimulation of peripheral acetylcholine receptors. In some embodiments, an anti-cholinergic agent is an agent which blocks the stimulation of central acetylcholine receptors. In some embodiments, an anti-cholinergic agent is an agent which blocks the stimulation of peripheral acetylcholine receptors, central acetylcholine receptors, or a combination thereof. In some embodiments, the anti-cholinergic agent is a non-brain penetrating anti-cholinergic agent. In some embodiments, the anti-cholinergic agent is an anti-cholinergic agent that does penetrate into the brain. In some embodiments, anti-cholinergic agents may include but are not limited to anti-muscarinic anti-cholinergic agents. In some embodiments, anti-cholinergic agents may include but are not limited to anti-nicotinic anti-cholinergic agents. In some embodiments, the anti-cholinergic agent is an anti-cholinergic agent that does not cause confusion or other cognitive problems. In some embodiments, the anti-cholinergic agent is an anti-cholinergic agent that does not cause dizziness. In some embodiments, the anti-cholinergic agent is an anti-cholinergic agent that does not cause blurred vision. In some embodiments, the anti-cholinergic agent is an anti-cholinergic agent that does not cause sedation. In some embodiments, the anti-cholinergic agent is an anti-cholinergic agent that does not cause balance and gait impairments. In some embodiments, the anti-cholinergic agent is an anti-cholinergic agent that does not increase the risk of falls. In some embodiments, the anti-cholinergic agent is selected from the group consisting of a quaternary ammonium anti-cholinergic muscarinic receptor antagonist, a quaternary ammonium non-selective peripheral Anti-Cholinergic agent, a sulfonium non-selective peripheral Anti-Cholinergic agent, a non-selective peripheral muscarinic anti-cholinergic agent, (1 S)-(3R)-1-azabicyclo[2.2.2]oct-3-yl 3,4-dihydro-1-phenyl-2(1H)-iso-quinolinecarboxylate (solifenacin) and its pharmaceutically acceptable salts, 1-methylpiperidin-4-yl) 2,2-di(phenyl)-2-propoxyacetate (propiverine) and its pharmaceutically acceptable salts, 1,4,5, 6-tetrahydro-1-methylpyrimidin-2-ylmethyl α-cyclohexyl-α-hydroxy-α-phenylacetate (oxyphencyclimine) and its pharmaceutically acceptable salts, (R)-N,N-diisopropyl-3-(2-hydroxy-5-methylphenyl)-3-phenylpropanamine (tolterodine) and its pharmaceutically acceptable salts. In some embodiments, the quaternary ammonium anti-cholinergic muscarinic receptor antagonist is selected from the group consisting of trospium and its pharmaceutically acceptable salts, and glycopyrrolate and its pharmaceutically acceptable salts. In some embodiments, the anti-cholinergic agent is a compound of formula I:

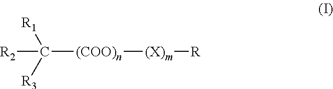

wherein R is a radical selected from the group consisting of those of formulas (a)-(e):

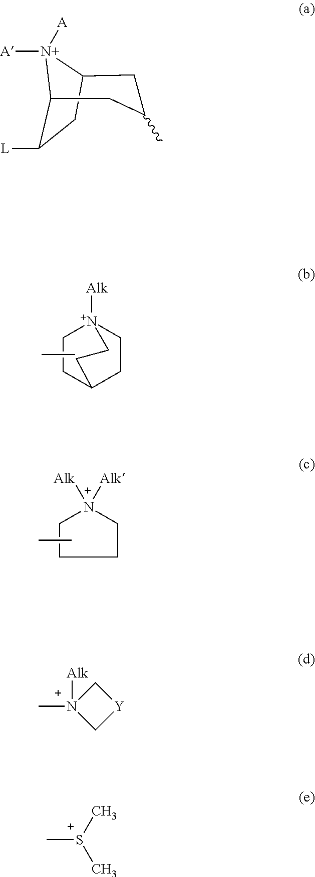

A being methyl and A′ being (C1-C4)alkyl or 2-fluoroethyl group or A and A′ forming a 1,4-butylene or 1,5-pentylene chain, L being hydrogen or methoxy, Alk and Alk′ each being (C1-C4)alkyl and Y being a bivalent radical selected from the group consisting of 1,2-ethylene, 1,3-propylene, 1,4-butylene and 2-oxa-1,3-propylene; the corresponding counter ion being a pharmaceutically acceptable anion, such as a chloro, bromo, iodo, tartrate, hydrogen tartrate, succinate, maleate, fumarate, sulfate, hydrogen sulfate or methylsulfate anion; n and m, independently, are zero or 1; X is a (C2-C3)alkylene group; R1 and R2 are each phenyl, cyclopentyl, cyclohexyl, 1-cyclohexenyl, 2-thienyl and, when R is a radical (a), also each represents (C1-C4)alkyl; R3 is H or OH or, only when R is a radical (a), also a COOAlk group, Alk being a (C1-C4)alkyl group.

In some embodiments, R=(a), A=A′=CH3, L=H; n=1; m=0; R1=R2=n-C3H7; and R3=H. In some embodiments, R=(a), A=CH3, A′=isopropyl, L=H; n=1; m=0; R1=phenyl; R2=cyclopentyl; and R3=H. In some embodiments, R=(a), A=CH₃, A′=2-fluoroethyl, L=H; n=1; m=0; R₁═R₂=phenyl; and R₃═OH. In some embodiments, R=(a), A=A′=CH₃, L=H; n=1; m=0; R=phenyl; and R₂═R₃═H. In some embodiments, R=(a), A=CH₃, A′=isopropyl, L=H; n=1; m=0; R₁═R₂=n-C₃H₇; and R₃═H. In some embodiments, R=(a), A=A′=CH₃, L=H; n=1; m=0; R₁=phenyl; R₂═COOC₂H₅; and R₃═H. In some embodiments, R=(a), A=A′=CH₃, L=methoxy; n=1; m=0; R₁═R₂=phenyl, and R₃═OH. In some embodiments, R=(a), A+A′=1,4-butylene, L=H; n=1; m=0; R₁═R₂=phenyl; and R₃═OH. In some embodiments, R=(b)-3-, Alk=methyl; n=1; m=0; R₁═R₂=phenyl; and R₃═OH. In some embodiments, R=(b)-3-, Alk=methyl; n=1; m=0; R₁=phenyl; R₂=cyclopentyl; and R₃═OH. In some embodiments, R=(c)-3-, both Alk and Alk′=ethyl; n=1; m=0; R₁═R₂=phenyl; and R₃═OH. In some embodiments, R=(c)-3-, both Alk and Alk′=methyl; n=1; m=0; R₁═R₂=phenyl; and R₃═OH. In some embodiments, R=(c)-3-, Alk=methyl and Alk′=ethyl; n=1; m=0; R₁=phenyl; R₂=cyclopentyl; and R₃═H. In some embodiments, R=(c)-3-, both Alk and Alk′=methyl; n=1; m=0; R₁=phenyl; R₂=cyclopentyl; and R₃═H. In some embodiments, R=(c)-3-, both Alk and Alk′=methyl n=1; m=0; R₁=phenyl; R₂=2-thienyl; and R₃═OH. In some embodiments, R=(c)-3-, both Alk and Alk′=methyl; n=1; m=0; R₁=phenyl; R₂=cyclohexyl; and R₃═H. In some embodiments, R=(c)-2-, both Alk and Alk′=methyl; n=1; m=1; X=1,2-ethylene; R₁=phenyl; R₂=cyclohexyl; and R₃═OH. In some embodiments, R=(c)-3-, both Alk and Alk′=methyl; n=1; m=0; R₁=phenyl; R₂=cyclopentyl; and R₃═OH. In some embodiments, R=(c)-3-, both Alk and Alk′=methyl; n=1; m=0; R₁=phenyl; R₂=cyclopentyl; R₃═OH. In some embodiments, R=(d), Alk=methyl, Y=1,2-ethylene; n=1; m=1; X=1,2-ethylene; R₁═R₂=phenyl; and R₃═OH. In some embodiments, R=(d), Alk=CH₃, Y=1,3-propylene; n=0; m=1; X=1,2-ethylene; R₁=phenyl; R₂=1-cyclohexenyl; and R₃═H. In some embodiments, R=(d), Alk=methyl, Y=1,2-ethylene; n=0; m=1; X=1,2-ethylene; R₁=phenyl; R₂=cyclohexyl; and R₃═OH. In some embodiments, R=(d), Alk=methyl, Y=2-oxa-1,3-propylene; n=0; m=1; X=1,2-ethylene; R₁=phenyl; R₂=2-thienyl; and R₃═OH. In some embodiments, R=(e); n=1; m=1; X=1,2-ethylene; R₁=phenyl; R₂=cyclohexyl; and R₃═H. In some embodiments, R=(e); n=1; m=1; X=1,2-ethylene; R₁=phenyl; R₂=cyclohexyl; and R₃═OH.

In some embodiments, the anti-cholinergic agent is selected from the group consisting of anisotropine methylbromide, ciclotropium bromide, flutropium bromide, homatropine methylbromide, sintropium bromide, tematropium metilsulfate, tropenziline bromide, trospium chloride, clidinium bromide, droclidinium bromide, benzilonium bromide, benzopyrronium bromide, cyclopyrronium bromide, glycopyrronium bromide (glycopyrrolate), heteronium bromide, hexopyrronium bromide, oxypyrronium bromide, ritropirronium bromide, etipirium iodide, fenclexonium methylsulfate, tricyclamol chloride (procyclidine methochloride), tiemonium iodide, hexasonium iodide, and oxysonium iodide. In some embodiments, the anti-cholinergic agent is selected from the group consisting of Azoniaspiro[3β-benziloyloxy-(1α,5α-nortropane-8,1′-pyrrolidine]chloride (A+A′=1,4-butylene) described in U.S. Pat. No. 3,480,626, known under its International Non-proprietary Name trospium chloride, the tartrate, maleate, fumarate and succinate salts of trospium, solifenacin, described in U.S. Pat. No. 6,017,927 and the compound thereof with succinic acid, propiverine, described in DD 106643, and the hydrochloride thereof, oxyphencyclimine, described in GB 795758, and the hydrochloride thereof, tolterodine, described in U.S. Pat. No. 5,382,600, and the hydrogen tartrate thereof. In some embodiments, the anti-cholinergic agent is selected from the group consisting of a pharmaceutically acceptable salt of trospium, especially trospium chloride, succinate, maleate, fumarate or tartrate, a pharmaceutically acceptable salt of solifenacin, especially its compound with succinic acid 1:1, a pharmaceutically acceptable salt of propiverine, especially its hydrochloride, a pharmaceutically acceptable salt of oxyphencyclimine, especially its hydrochloride or a pharmaceutically acceptable salt of tolterodine, especially its L-hydrogen tartrate. In some embodiments, suitable anticholinergic agents include, but are not limited to glycopyrrolate, solifenacin, clinidium, cimetidine, ranitidine, digoxin, scopolamine, dantrolene, chloriazepoxide, atropine, nifedipine, amantadine, propantheline, furosemide, amoxapine, trospium, paroxetine, disopyramide, hydroxyzine, fesoterodine, meclizinedicyclomine, darifenacin, paroxetine, disopyramide, hydroxyzine, diphenhydramine, orphenadrine, olanzapine, clozapine, chlorpheniramine, desipramine, doxepin, biperiden, oxybutynin, benzatropine, promethazine, imipramine, nortriptyline, protriptyline, prochlorperazine, cyclobenzaprine, trihexyphenidyl, cyproheptadine, clomipramine, amitriptyline, chlorpromazine, tolterodine, meclizine, dicyclomine, and thioridazine.

In some embodiments, the NMDA receptor antagonist is selected from the group consisting of memantine, amantadine, and ketamine. In some embodiments, the NMDA receptor antagonist is memantine. In some embodiments, the memantine or pharmaceutically acceptable salts, hydrates, polymorphs, or solvates thereof comprises memantine hydrochloride. In some embodiments, the therapeutically effective amount of memantine or pharmaceutically acceptable salts, hydrates, polymorphs, or solvates thereof is configured for immediate release, extended release, delayed release or any combination thereof.

In some embodiments, the acetylcholinesterase inhibitor is selected from the group consisting of donepezil, rivastigmine, galantamine, tacrine, physostigmine, pyridostigmine, neostigmine, icopezil, zanapezil, ipidacrine, phenserine, ambenonium, edrophonium, ladostigil, huperzine A, or pharmaceutically acceptable salts, hydrates, polymorphs, or solvates thereof. In some embodiments, the acetylcholinesterase inhibitor is donepezil or pharmaceutically acceptable salts, hydrates, polymorphs, or solvates thereof. In some embodiments, the therapeutically effective amount of donepezil or pharmaceutically acceptable salts, hydrates, polymorphs, or solvates thereof is configured for immediate release, extended release, delayed release, or any combination thereof. In some embodiments, the therapeutically effective amount of donepezil or pharmaceutically acceptable salts, hydrates, polymorphs, or solvates thereof is from about 0.001 mg to about 1,000 mg, about 0.001 mg to about 30 mg, or about 0.2 mg to about 138 mg. In some embodiments, the therapeutically effective amount of donepezil or pharmaceutically acceptable salts, hydrates, polymorphs, or solvates thereof is about 5 mg, 10 mg, or 23 mg.

### Compositions

Some embodiments herein are directed to compositions comprising: a therapeutically effective amount of a 5-HT₆ receptor antagonist; a therapeutically effective amount of an acetylcholinesterase inhibitor; a therapeutically effective amount of an anti-cholinergic agent; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; a therapeutically effective amount of donepezil or pharmaceutically acceptable salts, hydrates or solvates thereof a therapeutically effective amount of trospium or pharmaceutically acceptable salts, hydrates or solvates thereof; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; a therapeutically effective amount of donepezil or pharmaceutically acceptable salts, hydrates or solvates thereof a therapeutically effective amount of glycopyrrolate or pharmaceutically acceptable salts, hydrates or solvates thereof; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the composition is suitable for oral administration. In some embodiments, the composition is suitable for delivery through other routes of administration, including transdermal delivery via patch and/or intranasal delivery.

Some embodiments are directed to compositions comprising: a therapeutically effective amount of a 5-HT_{2A} inverse agonist; a therapeutically effective amount of an acetylcholinesterase inhibitor; a therapeutically effective amount of an anti-cholinergic agent; and at least one pharmaceutically acceptable excipient. In some embodiments, the composition comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the composition comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the composition comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the composition comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the composition comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the composition comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient In some embodiments, the composition is suitable for oral administration. In some embodiments, the composition is suitable for delivery through other routes of administration, including transdermal delivery via patch and/or intranasal delivery.

Some embodiments are directed to compositions comprising a therapeutically effective amount of a NMDA receptor antagonist; a therapeutically effective amount of an acetylcholinesterase inhibitor; a therapeutically effective amount of an anti-cholinergic agent; and at least one pharmaceutically acceptable excipient. In some embodiments, the composition comprises a therapeutically effective amount of memantine or pharmaceutically acceptable salts, hydrates or solvates thereof; a therapeutically effective amount of an donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the composition comprises a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the composition comprises a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the composition comprises a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the composition comprises a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the composition comprises a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the composition is suitable for oral administration. In some embodiments, the composition is suitable for delivery through other routes of administration, including transdermal delivery via patch and/or intranasal delivery.

Some embodiments herein are directed to compositions comprising: a therapeutically effective amount of a 5-HT₆ receptor antagonist; a 5-HT_{2A} inverse agonist; a therapeutically effective amount of an acetylcholinesterase inhibitor; a therapeutically effective amount of an anti-cholinergic agent; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the composition is suitable for oral administration. In some embodiments, the composition is suitable for delivery through other routes of administration, including transdermal delivery via patch and/or intranasal delivery.

Some embodiments herein are directed to compositions comprising: a therapeutically effective amount of a 5-HT₆ receptor antagonist; a NMDA receptor antagonist; a therapeutically effective amount of an acetylcholinesterase inhibitor; a therapeutically effective amount of an anti-cholinergic agent; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the composition comprises a therapeutically effective amount of 3-phenylsulfonyl-8-piperazinyl-1 yl-quinoline or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the composition comprises a therapeutically effective amount of 3-phenylsulfonyl-8-piperazinyl-1 yl-quinoline or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the composition is suitable for oral administration. In some embodiments, the composition is suitable for delivery through other routes of administration, including transdermal delivery via patch and/or intranasal delivery.

Some embodiments herein are directed to compositions comprising: a therapeutically effective amount of a 5-HT_{2A} inverse agonist; a NMDA receptor antagonist; a therapeutically effective amount of an acetylcholinesterase inhibitor; a therapeutically effective amount of an anti-cholinergic agent; and at least one pharmaceutically acceptable excipient. In some embodiments, the composition comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the composition comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the composition comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the composition comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the composition comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the composition comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the composition is suitable for oral administration. In some embodiments, the composition is suitable for delivery through other routes of administration, including transdermal delivery via patch and/or intranasal delivery.

Some embodiments are directed to compositions comprising a therapeutically effective amount of a 5-HT₆ receptor antagonist; a therapeutically effective amount of a 5-HT_{2A} inverse agonist; a therapeutically effective amount of a NMDA receptor antagonist; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; and at least one pharmaceutically acceptable excipient. In some embodiments, the composition is suitable for oral administration. In some embodiments, the composition is suitable for delivery through other routes of administration, including transdermal delivery via patch and/or intranasal delivery.

Some embodiments are directed to compositions comprising a therapeutically effective amount of a 5-HT₆ receptor antagonist; a therapeutically effective amount of a 5-HT_{2A} inverse agonist; a therapeutically effective amount of a NMDA receptor antagonist; a therapeutically effective amount of an acetylcholinesterase inhibitor; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of donepezil or pharmaceutically acceptable salts, hydrates or solvates thereof; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of rivastigmine or pharmaceutically acceptable salts, hydrates or solvates thereof; and at least one pharmaceutically acceptable excipient. In some embodiments, the composition is suitable for oral administration. In some embodiments, the composition is suitable for delivery through other routes of administration, including transdermal delivery via patch and/or intranasal delivery.

Some embodiments are directed to compositions comprising a therapeutically effective amount of a 5-HT₆ receptor antagonist; a therapeutically effective amount of a 5-HT_{2A} inverse agonist; a therapeutically effective amount of a NMDA receptor antagonist; a therapeutically effective amount of an anti-cholinergic agent; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the composition is suitable for oral administration. In some embodiments, the composition is suitable for delivery through other routes of administration, including transdermal delivery via patch and/or intranasal delivery.

Some embodiments herein are directed to compositions comprising: a therapeutically effective amount of a 5-HT₆ receptor antagonist; a 5-HT_{2A} inverse agonist; a NMDA receptor antagonist; a therapeutically effective amount of an acetylcholinesterase inhibitor; a therapeutically effective amount of an anti-cholinergic agent; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the composition comprises a therapeutically effective amount of 3-phenylsulfonyl-8-piperazinyl-1 yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the composition comprises a therapeutically effective amount of 3-phenylsulfonyl-8-piperazinyl-1 yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the composition comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the composition is suitable for oral administration. In some embodiments, the composition is suitable for delivery through other routes of administration, including transdermal delivery via patch and/or intranasal delivery.

In some embodiments, the 5-HT₆ receptor antagonist is 3-phenylsulfonyl-8-piperazinyl-1yl-quinoline or pharmaceutically acceptable salts, hydrates, polymorphs, or solvates thereof. In some embodiments, the therapeutically effective amount of 3-phenylsulfonyl-8-piperazinyl-1yl-quinoline or pharmaceutically acceptable salts, hydrates, polymorphs, or solvates thereof is from about 0.001 mg to about 1,000 mg, about 0.001 mg to about 200 mg, about 0.001 mg to about 175 mg, or 0.001 mg to about 70 mg. In some embodiments, the therapeutically effective amount of 3-phenylsulfonyl-8-piperazinyl-1yl-quinoline or pharmaceutically acceptable salts, hydrates, polymorphs, or solvates thereof is about 15 mg, about 35 mg, or about 70 mg. In some embodiments, the therapeutically effective amount of 3-phenylsulfonyl-8-piperazinyl-1yl-quinoline or pharmaceutically acceptable salts, hydrates, polymorphs, or solvates thereof is an amount selected from the group consisting of an amount of 3-phenylsulfonyl-8-piperazinyl-1yl-quinoline that may cause convulsions in a subject to which it is administered; an amount that would be expected to exceed the maximum tolerated dose for the subject to which it is administered; an amount associated with systemic exposures characterized by an AUCₜₐᵤ₋ₛₛ of about 8.2 μg·h/ml, a Cₘₐₓ of about 0.26 μg/ml; or a combination thereof an mount associated with systemic exposures characterized by an AUC, Cₘₐₓ, or combinations thereof, that are about 2 to about 3 times higher than the mean clinical exposure achieved at the proposed clinical dose for monotherapy with 3-phenylsulfonyl-8-piperazinyl-1yl-quinoline (i.e. mean AUCₜₐᵤ₋ₛₛ of about 3.2 μg·h/ml and Cₘₐₓ of about 0.180 μg/ml), an amount associated with a recorded systemic clinical exposure that is greater than the highest recorded systemic clinical exposure (AUC_{0-∞} of about 9.25 μg·h/ml and Cₘₐₓ of about 0.293 μg/ml), an amount of 3-phenylsulfonyl-8-piperazinyl-1yl-quinoline that is greater than about 10 mg/kg/day, an amount of 3-phenylsulfonyl-8-piperazinyl-1yl-quinoline that is greater than 15 mg/day, a dose of 3-phenylsulfonyl-8-piperazinyl-1yl-quinoline that is greater than about 35 mg/day or any combination thereof. In some embodiments, the therapeutically effective amount of 3-phenylsulfonyl-8-piperazinyl-1yl-quinoline or pharmaceutically acceptable salts, hydrates, polymorphs, or solvates thereof is configured for immediate release, extended release, delayed release, or any combination thereof.

In some embodiments, the 5-HT_{2A} inverse agonist is 1-[3-(4-bromo-2-methyl-2H-pyrazol-3-yl)-4-methoxy-phenyl]-3-(2,4-difluoro-phenyl)-urea. In some embodiments, the therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates, polymorphs, or solvates thereof is from about 0.001 mg to about 1,000 mg, about 0.001 mg to about 200 mg, or about 0.001 mg to about 160 mg. In some embodiments, the therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates, polymorphs, or solvates thereof is about 20 mg, about 40 mg, about 80 mg, or about 160 mg. In some embodiments, the therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates, polymorphs, or solvates thereof is configured for immediate release, extended release, delayed release, or any combination thereof.

In some embodiments, the NMDA receptor antagonist is selected from the group consisting of memantine, amantadine, and ketamine. In some embodiments, the NMDA receptor antagonist is memantine or pharmaceutically acceptable salts, hydrates, polymorphs, or solvates thereof. In some embodiments, the therapeutically effective amount of memantine or pharmaceutically acceptable salts, hydrates, polymorphs, or solvates thereof is from about 0.001 mg to about 1,000 mg, or about 0.001 mg to about 30 mg. In some embodiments, the therapeutically effective amount of memantine or pharmaceutically acceptable salts, hydrates, polymorphs, or solvates thereof is about 5 mg, about 7 mg, about 10 mg, about 14 mg, about 20 mg, about 21 mg, or about 28 mg. In some embodiments, the therapeutically effective amount of memantine or pharmaceutically acceptable salts, hydrates, polymorphs, or solvates thereof is configured for immediate release, extended release, delayed release, or any combination thereof.

In some embodiments, the acetylcholinesterase inhibitor is selected from the group consisting of donepezil, rivastigmine, galantamine, tacrine, physostigmine, pyridostigmine, neostigmine, icopezil, zanapezil, ipidacrine, phenserine, ambenonium, edrophonium, ladostigil, huperzine A, pyridostigmine, ambenonium, demecarium, a phenanthrene derivative, caffeine, a piperidine tacrine (also known as tetrahydroaminoacridine), edrophonium, ladostigil, ungeremine, lactucopicrin, 6-[(3-cyclobutyl-2,3,4,5-tetrahydro-1H-3-benzazepin-7-yl)oxy]-N-methyl-3-pyridinecarboxamide hydrochloride or 1-{6-[(3-cyclobutyl-2,3,4,5-tetrahydro-1H-3-benzazepin-7-yl)oxy]-3-pyridinyl}-2-pyrrolidinone or pharmaceutically acceptable salts, hydrates, polymorphs, or solvates thereof.

In some embodiments, the acetylcholinesterase inhibitor is galantamine. In some embodiments, galantamine or pharmaceutically acceptable salts, hydrates, polymorphs, or solvates thereof is galantamine hydrobromide. In some embodiments, the therapeutically effective amount of galantamine or pharmaceutically acceptable salts, hydrates, polymorphs, or solvates thereof is configured for immediate release, extended release, delayed release, or any combination thereof. In some embodiments, the therapeutically effective amount of galantamine or pharmaceutically acceptable salts, hydrates, polymorphs, or solvates thereof is from about 0.001 mg to about 1,000 mg, or about 0.001 mg to about 30 mg. In some embodiments, the therapeutically effective amount of galantamine or pharmaceutically acceptable salts, hydrates, polymorphs, or solvates thereof is about 4 mg, about 8 mg, about 12 mg, about 16 mg, or about 24 mg. In some embodiments, the therapeutically effective amount of galantamine is about 1.001 to about 1,000 times greater than a recommended maximal dose level approved by the U.S. FDA. In some embodiments, the therapeutically effective amount of galantamine is about 1.5 to about 4 times greater than a recommended maximal dose level approved by the U.S. FDA. For Example, the dose of galantamine may be from about 36 mg to about 96 mg.

In some embodiments, the acetylcholinesterase inhibitor is tacrine. In some embodiments, tacrine or pharmaceutically acceptable salts, hydrates, polymorphs, or solvates thereof is tacrine hydrochloride. In some embodiments, the therapeutically effective amount of tacrine or pharmaceutically acceptable salts, hydrates, polymorphs, or solvates thereof is configured for immediate release, extended release, delayed release, or any combination thereof. In some embodiments, the therapeutically effective amount of tacrine or pharmaceutically acceptable salts, hydrates, polymorphs, or solvates thereof is from about 0.001 mg to about 1,000 mg, about 0.001 mg to about 640 mg. about 0.001 mg to about 160, or about 0.001 mg to about 120 mg In some embodiments, the therapeutically effective amount of tacrine or pharmaceutically acceptable salts, hydrates, polymorphs, or solvates thereof is about 10 mg, about 20 mg, about 30 mg, about 40 mg, about 120 mg, or about 160 mg. In some embodiments, the therapeutically effective amount of tacrine is about 1.001 to about 1,000 times greater than a recommended maximal dose level approved by the U.S. FDA. In some embodiments, the therapeutically effective amount of tacrine is about 1.5 to about 4 times greater than a recommended maximal dose level approved by the U.S. FDA. For Example, the dose of tacrine may be from about 240 mg to about 640 mg.

In some embodiments, the acetylcholinesterase inhibitor is donepezil or pharmaceutically acceptable salts, hydrates, polymorphs, or solvates thereof. In some embodiments, the acetylcholinesterase inhibitor is donepezil hydrochloride. In some embodiments, the therapeutically effective amount of donepezil or pharmaceutically acceptable salts, hydrates, polymorphs, or solvates thereof is configured for immediate release, extended release, delayed release, or any combination thereof. In some embodiments, the therapeutically effective amount of donepezil or pharmaceutically acceptable salts, hydrates, polymorphs, or solvates thereof is from about 0.001 mg to about 1,000 mg, about 0.001 mg to about 30 mg, or about 34.5 mg to about 92 mg. In some embodiments, the therapeutically effective amount of donepezil or pharmaceutically acceptable salts, hydrates, polymorphs, or solvates thereof is about 5 mg, 10 mg, or 23 mg. In some embodiments, the therapeutically effective amount of donepezil is about 1.001 to about 1,000 times greater than a recommended maximal dose level approved by the U.S. FDA. In some embodiments, the therapeutically effective amount of donepezil is about 1.5 to about 4 times greater than a recommended maximal dose level approved by the U.S. FDA. For Example, the dose of donepezil may be from about 34.5 mg to about 92 mg.

In some embodiments, the acetylcholinesterase inhibitor is rivastigmine or pharmaceutically acceptable salts, hydrates, polymorphs, or solvates thereof. In some embodiments, the therapeutically effective amount of rivastigmine or pharmaceutically acceptable salts, hydrates, polymorphs, or solvates thereof is configured for immediate release, for extended release, delayed release, or any combination thereof. In some embodiments, the therapeutically effective amount of rivastigmine or pharmaceutically acceptable salts, hydrates, polymorphs, or solvates thereof is from about 0.001 mg to about 1,000 mg, about 0.001 mg to about 48 mg, about 12 mg to about 48 mg, about 19 mg to about 54 mg or about 0.001 mg to about 60 mg. In some embodiments, the therapeutically effective amount of rivastigmine or pharmaceutically acceptable salts, hydrates, polymorphs, or solvates thereof is about 1.5 mg, about 3 mg, about 4.5 mg, about 6 mg, about 9 mg, about 9.5 mg, about 12 mg, about 13.3 mg, about 24 mg, or about 48 mg. In some embodiments, the therapeutically effective amount of rivastigmine is about 1.001 to about 1,000 times greater than a recommended maximal dose level approved by the U.S. FDA. In some embodiments, the therapeutically effective amount of rivastigmine is about 1.5 to about 4 times greater than a recommended maximal dose level approved by the U.S. FDA. For Example, the dose of rivastigmine may be from about 18 mg to about 48 mg.

In some embodiments, the anti-cholinergic agent is selected from the group consisting of quaternary ammonium anti-cholinergic muscarinic receptor antagonist, a quaternary ammonium non-selective peripheral Anti-Cholinergic agent, a sulfonium non-selective peripheral Anti-Cholinergic agent, a non-selective peripheral muscarinic anti-cholinergic agent, (1 S)-(3R)-1-azabicyclo[2.2.2]oct-3-yl 3,4-dihydro-1-phenyl-2(1H)-iso-quinolinecarboxylate (solifenacin) and its pharmaceutically acceptable salts, 1-methylpiperidin-4-yl) 2,2-di(phenyl)-2-propoxyacetate (propiverine) and its pharmaceutically acceptable salts, 1,4,5,6-tetrahydro-1-methylpyrimidin-2-ylmethyl α-cyclohexyl-α-hydroxy-α-phenylacetate (oxyphencyclimine) and its pharmaceutically acceptable salts, (R)-N,N-diisopropyl-3-(2-hydroxy-5-methylphenyl)-3-phenylpropanamine (tolterodine) and its pharmaceutically acceptable salts.

In some embodiments, the quaternary ammonium anti-cholinergic muscarinic receptor antagonist is selected from trospium and glycopyrrolate. In some embodiments, the quaternary ammonium anti-cholinergic muscarinic receptor antagonist is glycopyrrolate. In some embodiments, the therapeutically effective amount of glycopyrrolate is from about 0.1 mg to about 10.0 mg. In some embodiments, the therapeutically effective amount of glycopyrrolate is from about 0.1 mg to about 0.8 mg. In some embodiments, the therapeutically effective amount of glycopyrrolate is from about 2.0 mg to about 4.0 mg. In some embodiments, the therapeutically effective amount of glycopyrrolate is from about 3.0 mg to about 10.0 mg. In some embodiments, the therapeutically effective amount of glycopyrrolate is about 1.0 mg. In some embodiments, the therapeutically effective amount of glycopyrrolate is an amount from about 20% to about 600% of the amount of glycopyrrolate that is currently administered for anti-cholinergic therapy. In some embodiments, the therapeutically effective amount of glycopyrrolate is an amount from about 0.2 mg to about 60 mg of the amount of glycopyrrolate that is currently administered for anti-cholinergic therapy. In some embodiments, the quaternary ammonium anti-cholinergic muscarinic receptor antagonist is trospium or pharmaceutically acceptable salts, hydrates or solvates thereof. In some embodiments, the quaternary ammonium anti-cholinergic muscarinic receptor antagonist is trospium chloride. In some embodiments, the therapeutically effective amount of trospium or pharmaceutically acceptable salts, hydrates or solvates thereof is from about 0.1 mg to about 120 mg. In some embodiments, the therapeutically effective amount of trospium or pharmaceutically acceptable salts, hydrates or solvates thereof is about 20 mg, about 40 mg, or about 60 mg. In some embodiments, the therapeutically effective amount of trospium or pharmaceutically acceptable salts, hydrates or solvates thereof is about 20 mg per day, about 40 mg per day, or about 60 mg per day. In some embodiments, the therapeutically effective amount of trospium is an amount from about 20% to about 600% of the amount of trospium or pharmaceutically acceptable salts, hydrates or solvates thereof that is currently administered for anti-cholinergic therapy.

In some embodiments, the non-selective peripheral muscarinic anti-cholinergic agent is solifenacin. In some embodiments, the therapeutically effective amount of solifenacin is an amount from about 20% to about 600% of the amount of solifenacin that is currently administered for anti-cholinergic therapy. In some embodiments, the therapeutically effective amount of solifenacin is an amount from about 1 mg to about 30 mg.

In some embodiments, the anti-cholinergic agent is a compound of formula I:

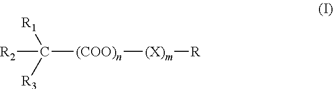

wherein R is a radical selected from the group consisting of those of formulas (a)-(e):

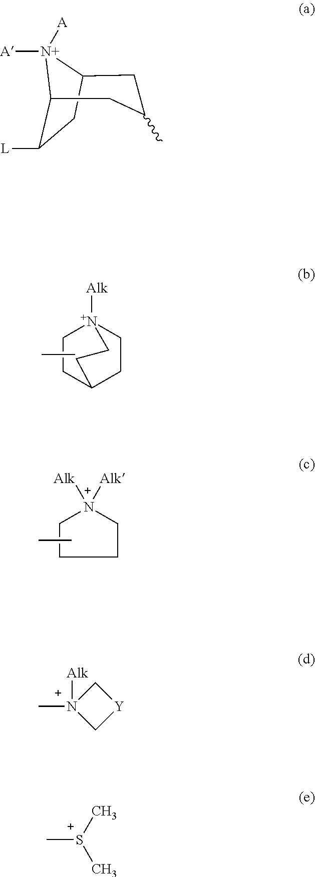

A being methyl and A′ being (C1-C4)alkyl or 2-fluoroethyl group or A and A′ forming a 1,4-butylene or 1,5-pentylene chain, L being hydrogen or methoxy, Alk and Alk′ each being (C1-C4)alkyl and Y being a bivalent radical selected from the group consisting of 1,2-ethylene, 1,3-propylene, 1,4-butylene and 2-oxa-1,3-propylene; the corresponding counter ion being a pharmaceutically acceptable anion, such as a chloro, bromo, iodo, tartrate, hydrogen tartrate, succinate, maleate, fumarate, sulfate, hydrogen sulfate or methylsulfate anion;
n and m, independently, are zero or 1;
X is a (C2-C3)alkylene group;
R1 and R2 are each phenyl, cyclopentyl, cyclohexyl, 1-cyclohexenyl, 2-thienyl and, when R is a radical (a), also each represents (C1-C4)alkyl;
R3 is H or OH or, only when R is a radical (a), also a COOAlk group, Alk being a (C1-C4)alkyl group.

In some embodiments, R=(a), A=A′=CH3, L=H; n=1; m=0; R1=R2=n-C3H7; and R3=H. In some embodiments, R=(a), A=CH3, A′=isopropyl, L=H; n=1; m=0; R1=phenyl; R2=cyclopentyl; and R3=H. In some embodiments, R=(a), A=CH₃, A′=2-fluoroethyl, L=H; n=1; m=0; R₁═R₂=phenyl; and R₃═OH. In some embodiments, R=(a), A=A′=CH₃, L=H; n=1; m=0; R=phenyl; and R₂═R₃═H. In some embodiments, R=(a), A=CH₃, A′=isopropyl, L=H; n=1; m=0; R₁═R₂=n-C₃H₇; and R₃═H. In some embodiments, R=(a), A=A′=CH₃, L=H; n=1; m=0; R₁=phenyl; R₂═COOC₂H₅; and R₃═H. In some embodiments, R=(a), A=A′=CH₃, L=methoxy; n=1; m=0; R₁═R₂=phenyl, and R₃═OH. In some embodiments, R=(a), A+A′=1,4-butylene, L=H; n=1; m=0; R₁═R₂=phenyl; and R₃═OH. In some embodiments, R=(b)-3-, Alk=methyl; n=1; m=0; R₁═R₂=phenyl; and R₃═OH. In some embodiments, R=(b)-3-, Alk=methyl; n=1; m=0; R₁=phenyl; R₂=cyclopentyl; and R₃═OH. In some embodiments, R=(c)-3-, both Alk and Alk′=ethyl; n=1; m=0; R₁═R₂=phenyl; and R₃═OH. In some embodiments, R=(c)-3-, both Alk and Alk′=methyl; n=1; m=0; R₁═R₂=phenyl; and R₃═OH. In some embodiments, R=(c)-3-, Alk=methyl and Alk′=ethyl; n=1; m=0; R₁=phenyl; R₂=cyclopentyl; and R₃═H. In some embodiments, R=(c)-3-, both Alk and Alk′=methyl; n=1; m=0; R₁=phenyl; R₂=cyclopentyl; and R₃═H. In some embodiments, R=(c)-3-, both Alk and Alk′=methyl n=1; m=0; R₁=phenyl; R₂=2-thienyl; and R₃═OH. In some embodiments, R=(c)-3-, both Alk and Alk′=methyl; n=1; m=0; R₁=phenyl; R₂=cyclohexyl; and R₃═H. In some embodiments, R=(c)-2-, both Alk and Alk′=methyl; n=1; m=1; X=1,2-ethylene; R₁=phenyl; R₂=cyclohexyl; and R₃═OH. In some embodiments, R=(c)-3-, both Alk and Alk′=methyl; n=1; m=0; R₁=phenyl; R₂=cyclopentyl; and R₃═OH. In some embodiments, R=(c)-3-, both Alk and Alk′=methyl; n=1; m=0; R₁=phenyl; R₂=cyclopentyl; R₃═OH. In some embodiments, R=(d), Alk=methyl, Y=1,2-ethylene; n=1; m=1; X=1,2-ethylene; R₁═R₂=phenyl; and R₃═OH. In some embodiments, R=(d), Alk=CH₃, Y=1,3-propylene; n=0; m=1; X=1,2-ethylene; R₁=phenyl; R₂=1-cyclohexenyl; and R₃═H. In some embodiments, R=(d), Alk=methyl, Y=1,2-ethylene; n=0; m=1; X=1,2-ethylene; R₁=phenyl; R₂=cyclohexyl; and R₃═OH. In some embodiments, R=(d), Alk=methyl, Y=2-oxa-1,3-propylene; n=0; m=1; X=1,2-ethylene; R₁=phenyl; R₂=2-thienyl; and R₃═OH. In some embodiments, R=(e); n=1; m=1; X=1,2-ethylene; R₁=phenyl; R₂=cyclohexyl; and R₃═H. In some embodiments, R=(e); n=1; m=1; X=1,2-ethylene; R₁=phenyl; R₂=cyclohexyl; and R₃═OH.

In some embodiments, the anti-cholinergic agent is selected from the group consisting of anisotropine methylbromide, ciclotropium bromide, flutropium bromide, homatropine methylbromide, sintropium bromide, tematropium metilsulfate, tropenziline bromide, trospium chloride, clidinium bromide, droclidinium bromide, benzilonium bromide, benzopyrronium bromide, cyclopyrronium bromide, glycopyrronium bromide (glycopyrrolate), heteronium bromide, hexopyrronium bromide, oxypyrronium bromide, ritropirronium bromide, etipirium iodide, fenclexonium methylsulfate, tricyclamol chloride (procyclidine methochloride), tiemonium iodide, hexasonium iodide, and oxysonium iodide.

In some embodiments, the anti-cholinergic agent is selected from the group consisting of Azoniaspiro[3β-benziloyloxy-(1α,5α-nortropane-8,1′-pyrrolidine]chloride (formula II, A+A′=1,4-butylene) described in U.S. Pat. No. 3,480,626, known under its International Non-proprietary Name trospium chloride, the tartrate, maleate, fumarate and succinate salts of trospium, solifenacin, described in U.S. Pat. No. 6,017,927 and the compound thereof with succinic acid, propiverine, described in DD 106643, and the hydrochloride thereof, oxyphencyclimine, described in GB 795758, and the hydrochloride thereof, tolterodine, described in U.S. Pat. No. 5,382,600, and the hydrogen tartrate thereof.

In some embodiments, the anti-cholinergic agent is selected from the group consisting of a pharmaceutically acceptable salt of trospium, especially trospium chloride, succinate, maleate, fumarate or tartrate, a pharmaceutically acceptable salt of solifenacin, especially its compound with succinic acid 1:1, a pharmaceutically acceptable salt of propiverine, especially its hydrochloride, a pharmaceutically acceptable salt of oxyphencyclimine, especially its hydrochloride or a pharmaceutically acceptable salt of tolterodine, especially its L-hydrogen tartrate. In some embodiments, suitable anticholinergic agents include, but are not limited to clinidium, cimetidine, ranitidine, digoxin, scopolamine, dantrolene, chlordiazepoxide, atropine, nifedipine, amantadine, propantheline, propantheline, furosemide, amoxapine, paroxetine, disopyramide, hydroxyzine, diphenhydramine, orphenadrine, olanzapine, clozapine, chlorpheniramine, desipramine, doxepin, biperiden, oxybutynin, benzatropine, promethazine, imipramine, nortriptyline, protriptyline, prochlorperazine, cyclobenzaprine, trihexyphenidyl, cyproheptadine, clomipramine, amitriptyline, chlorpromazine, tolterodine, meclizine, dicyclomine, and thioridazine.

Some embodiments are directed to a composition comprising: a therapeutically effective amount of 3-phenylsulfonyl-8-piperazinyl-1yl-quinoline or pharmaceutically acceptable salts, hydrates or solvates thereof; a therapeutically effective amount of an acetylcholinesterase inhibitor; a therapeutically effective amount of an anti-cholinergic agent; and at least one pharmaceutically acceptable excipient. In some embodiments, the composition is suitable for oral administration.

In some embodiments, the therapeutically effective amount of 3-phenylsulfonyl-8-piperazinyl-1yl-quinoline or pharmaceutically acceptable salts, hydrates, polymorphs, or solvates thereof is from about 0.001 mg to about 1,000 mg, about 0.001 mg to about 200 mg, about 0.001 mg to about 175 mg, or 0.001 mg to about 70 mg. In some embodiments, the therapeutically effective amount of 3-phenylsulfonyl-8-piperazinyl-1yl-quinoline or pharmaceutically acceptable salts, hydrates, polymorphs, or solvates thereof is about 15 mg, about 35 mg, or about 70 mg. In some embodiments, the therapeutically effective amount of 3-phenylsulfonyl-8-piperazinyl-1yl-quinoline or pharmaceutically acceptable salts, hydrates, polymorphs, or solvates thereof is an amount selected from the group consisting of an amount of 3-phenylsulfonyl-8-piperazinyl-1yl-quinoline that may cause convulsions in a subject to which it is administered; an amount that would be expected to exceed the maximum tolerated dose for the subject to which it is administered; an amount associated with systemic exposures characterized by an AUCₜₐᵤ₋ₛₛ of about 8.2 μg·h/ml, a Cₘₐₓ of about 0.26 μg/ml; or a combination thereof an mount associated with systemic exposures characterized by an AUC, Cₘₐₓ, or combinations thereof, that are about 2 to about 3 times higher than the mean clinical exposure achieved at the proposed clinical dose for monotherapy with 3-phenylsulfonyl-8-piperazinyl-1yl-quinoline (i.e. mean AUCₜₐᵤ₋ₛₛ of about 3.2 μg·h/ml and Cₘₐₓ of about 0.180 μg/ml), an amount associated with a recorded systemic clinical exposure that is greater than the highest recorded systemic clinical exposure (AUC_{0-∞} of about 9.25 μg·h/ml and Cₘₐₓ of about 0.293 μg/ml), an amount of 3-phenylsulfonyl-8-piperazinyl-1yl-quinoline that is greater than about 10 mg/kg/day, an amount of 3-phenylsulfonyl-8-piperazinyl-1yl-quinoline that is greater than 15 mg/day, a dose of 3-phenylsulfonyl-8-piperazinyl-1yl-quinoline that is greater than about 35 mg/day or any combination thereof.

In some embodiments, the acetylcholinesterase inhibitor is selected from the group consisting of donepezil, rivastigmine, galantamine, tacrine, physostigmine, pyridostigmine, neostigmine, icopezil, zanapezil, ipidacrine, phenserine, ambenonium, edrophonium, ladostigil, huperzine A, or pharmaceutically acceptable salts, hydrates, polymorphs, or solvates thereof. In some embodiments, the acetylcholinesterase inhibitor is donepezil or pharmaceutically acceptable salts, hydrates, polymorphs, or solvates thereof. In some embodiments, the therapeutically effective amount of donepezil or pharmaceutically acceptable salts, hydrates, polymorphs, or solvates thereof is configured for immediate release, extended release, delayed release, or any combination thereof. In some embodiments, the therapeutically effective amount of donepezil or pharmaceutically acceptable salts, hydrates, polymorphs, or solvates thereof is from about 0.001 mg to about 1,000 mg, about 0.001 mg to about 30 mg, or about 0.2 mg to about 138 mg. In some embodiments, wherein the therapeutically effective amount of donepezil or pharmaceutically acceptable salts, hydrates, polymorphs, or solvates thereof is about 5 mg, 10 mg, or 23 mg.

In some embodiments, the anti-cholinergic agent is selected from the group consisting of quaternary ammonium anti-cholinergic muscarinic receptor antagonist, a quaternary ammonium non-selective peripheral Anti-Cholinergic agent, a sulfonium non-selective peripheral Anti-Cholinergic agent, a non-selective peripheral muscarinic anti-cholinergic agent, (1 S)-(3R)-1-azabicyclo[2.2.2]oct-3-yl 3,4-dihydro-1-phenyl-2(1H)-iso-quinolinecarboxylate (solifenacin) and its pharmaceutically acceptable salts, 1-methylpiperidin-4-yl) 2,2-di(phenyl)-2-propoxyacetate (propiverine) and its pharmaceutically acceptable salts, 1,4,5, 6-tetrahydro-1-methylpyrimidin-2-ylmethyl α-cyclohexyl-α-hydroxy-α-phenylacetate (oxyphencyclimine) and its pharmaceutically acceptable salts, (R)-N,N-diisopropyl-3-(2-hydroxy-5-methylphenyl)-3-phenylpropanamine (tolterodine) and its pharmaceutically acceptable salts.

In some embodiments, the quaternary ammonium anti-cholinergic muscarinic receptor antagonist is selected from trospium and glycopyrrolate or pharmaceutically acceptable salts, hydrates or solvates thereof. In some embodiments, the quaternary ammonium anti-cholinergic muscarinic receptor antagonist is trospium or pharmaceutically acceptable salts, hydrates or solvates thereof. In some embodiments, the quaternary ammonium anti-cholinergic muscarinic receptor antagonist is trospium chloride. In some embodiments, the therapeutically effective amount of trospium or pharmaceutically acceptable salts, hydrates or solvates thereof is from about 0.1 mg to about 120 mg. In some embodiments, the therapeutically effective amount of trospium or pharmaceutically acceptable salts, hydrates or solvates thereof is about 20 mg, about 40 mg, or about 60 mg. In some embodiments, the therapeutically effective amount of trospium or pharmaceutically acceptable salts, hydrates or solvates thereof is about 20 mg per day, about 40 mg per day, or about 60 mg per day. In some embodiments, the therapeutically effective amount of trospium is an amount from about 20% to about 600% of the amount of trospium or pharmaceutically acceptable salts, hydrates or solvates thereof that is currently administered for anti-cholinergic therapy.

In some embodiments any of the compositions described herein may further comprise at least one additional therapeutic agent.

In some embodiments, the least one additional therapeutic agent is an NMDA receptor antagonist. In some embodiments, the NMDA receptor antagonist is selected from the group consisting of memantine, amantadine, and ketamine. In some embodiments, the NMDA receptor antagonist is memantine. In some embodiments, the memantine or pharmaceutically acceptable salts, hydrates, polymorphs, or solvates thereof comprises memantine hydrochloride. In some embodiments, the therapeutically effective amount of memantine or pharmaceutically acceptable salts, hydrates, polymorphs, or solvates thereof is configured for extended release, delayed release or any combination thereof. In some embodiments, the therapeutically effective amount of memantine or pharmaceutically acceptable salts, hydrates, polymorphs, or solvates thereof is from about 0.001 mg to about 1,000 mg, or about 0.001 mg to about 30 mg. In some embodiments, the therapeutically effective amount of memantine or pharmaceutically acceptable salts, hydrates, polymorphs, or solvates thereof is about 5 mg, about 7 mg, about 10 mg, about 14 mg, about 20 mg, about 21 mg, or about 28 mg. In some embodiments, memantine or pharmaceutically acceptable salts, hydrates or solvates thereof is administered to a subject in need thereof in an amount that is considered to be sub therapeutic. In some embodiments, the NMDA receptor antagonist is amantadine. In some embodiments, the amantadine or pharmaceutically acceptable salts, hydrates, polymorphs, or solvates thereof comprises amantadine hydrochloride. In some embodiments, the therapeutically effective amount of amantadine or pharmaceutically acceptable salts, hydrates, polymorphs, or solvates thereof is configured for immediate release, extended release, delayed release, or any combination thereof. In some embodiments, the therapeutically effective amount of amantadine or pharmaceutically acceptable salts, hydrates, polymorphs, or solvates thereof is from about 0.001 mg to about 1,000 mg, or about 0.001 mg to about 500 mg. In some embodiments, amantadine or pharmaceutically acceptable salts, hydrates or solvates thereof is administered to a subject in need thereof in an amount that is considered to sub therapeutic. In some embodiments, the therapeutically effective amount of amantadine or pharmaceutically acceptable salts, hydrates, polymorphs, or solvates thereof is from about 100 mg to about 400 mg. In some embodiments, the therapeutically effective amount of amantadine or pharmaceutically acceptable salts, hydrates, polymorphs, or solvates thereof is about 100 mg, 200 mg, 300 mg or about 400 mg.

In some embodiments, the least one additional therapeutic agent is a 5-HT_{2A} inverse agonist. In some embodiments, the 5-HT_{2A} inverse agonist is nelotanserin, pimavanserin, pruvanserin, eplivanserin, volinanserin, glemanserin, ketanserin, ritanserin, clozapine, or pharmaceutically acceptable salts, hydrates, polymorphs, or solvates thereof. In some embodiments, the nelotanserin or pharmaceutically acceptable salts, hydrates, polymorphs, or solvates thereof is 1-[3-(4-bromo-2-methyl-2H-pyrazol-3-yl)-4-methoxy-phenyl]-3-(2,4-difluoro-phenyl)-urea. In some embodiments, the 5-HT_{2A} inverse agonist is administered to a subject in need thereof in an amount that is considered to sub therapeutic. In some embodiments, the therapeutically effective amount of nelotanserin or pharmaceutically acceptable salts, hydrates, polymorphs, or solvates thereof is configured for immediate release, extended release, delayed release, or any combination thereof. In some embodiments, the therapeutically effective amount of nelotanserin or pharmaceutically acceptable salts, hydrates, polymorphs, or solvates thereof is from about 0.001 mg to about 1,000 mg, or about 0.001 mg to about 100 mg. In some embodiments, nelotanserin or pharmaceutically acceptable salts, hydrates or solvates thereof is administered to a subject in need thereof in an amount that is considered to sub therapeutic. In some embodiments, the therapeutically effective amount of nelotanserin or pharmaceutically acceptable salts, hydrates, polymorphs, or solvates thereof is about 20 mg, about 40 mg, or about 80 mg.

In some embodiments, the at least one additional therapeutic agent is a lithium compound or pharmaceutically acceptable salts, hydrates, polymorphs or solvates thereof. In some embodiments, the therapeutically effective amount of a lithium compound or pharmaceutically acceptable salts, hydrates, polymorphs, or solvates thereof is configured for extended release, delayed release, or any combination thereof. In some embodiments, the therapeutically effective amount of a lithium compound or pharmaceutically acceptable salts, hydrates, polymorphs or solvates thereof, is from about 0.001 mg to about 1000 mg, from about 0.001 mg to about 500 mg, from about 0.001 mg to about 100 mg, from about 0.001 mg to about 50 mg, from about 0.001 mg to about 10 mg, from about 0.001 mg to about 1 mg, from about 0.001 mg to about 0.1 mg, or from about 0.001 mg to about 0.01 mg. In some embodiments, the therapeutically effective amount of a lithium compound or pharmaceutically acceptable salts, hydrates, polymorphs or solvates thereof, is about 0.01 mg, about 0.1 mg, about 1 mg, about 5 mg, or about 10 mg. In some embodiments, the lithium compound is present in a sub therapeutically effective amount. In some embodiments, the sub therapeutically effective amount of a lithium compound or pharmaceutically acceptable salts, hydrates, polymorphs or solvates thereof, is an amount resulting in a serum concentration of between about 0.4 mM and about 1.6 mM, below about 0.4 mM, below about 0.5 mM, below about 0.4 mM, below about 0.3 mM, below about 0.2 mM, below about 0.1 mM, or below about 0.05 mM when administered to a subject. In some embodiments, the therapeutically effective amount of a lithium compound or pharmaceutically acceptable salts, hydrates, polymorphs, or solvates thereof is configured for extended release, delayed release, or any combination thereof.

In some embodiments, the at least one additional therapeutic agent is levodopa or pharmaceutically acceptable salts, hydrates, polymorphs, or solvates thereof. In some embodiments, the therapeutically effective amount of levodopa or pharmaceutically acceptable salts, hydrates, polymorphs, or solvates thereof is configured for immediate release, extended release, delayed release, or any combination thereof. In some embodiments, the therapeutically effective amount of levodopa or pharmaceutically acceptable salts, hydrates, polymorphs, or solvates thereof is from about 0.001 mg to about 10,000 mg, or about 0.001 mg to about 8,000 mg. In some embodiments, the therapeutically effective amount of levodopa or pharmaceutically acceptable salts, hydrates, polymorphs, or solvates thereof is about 285 mg, about 300 mg, about 400 mg, about 435 mg, 500 mg, about 585 mg, about 600 mg, about 700 mg, about 735 mg, about 750 mg, about 800 mg, about 980 mg, about 1,000 mg, about 1,225 mg, about 1,250 mg, about 1,470 mg, about 1,500 mg, about 1,715 mg, about 1,750 mg, about 1,960 mg, about 2,000 mg, about 2,205 mg, about 2,250 mg, about 2,450 mg, about 2,500 mg, about 2,750 mg, about 3,000 mg, about 3,250 mg, about 3,500 mg, about 3,750 mg, about 4,000 mg, about 4,250 mg, about 5,000 mg, about 5,250 mg, about 5,500 mg, about 5,750 mg, about 6,000 mg, about 6,250 mg, about 6,500 mg, about 6,750 mg, about 7,000 mg, about 7,250 mg, about 7,500 mg, about 7,750 mg, or about 8,000 mg. In some embodiments, the at least one additional therapeutic agent useful for treating a neurodegenerative disease is levodopa or pharmaceutically acceptable salts, hydrates, polymorphs, or solvates thereof and carbidopa or pharmaceutically acceptable salts, hydrates, polymorphs, or solvates thereof. In some embodiments, the therapeutically effective amount of levodopa further comprises carbidopa. In some embodiments, the therapeutically effective amount of carbidopa or pharmaceutically acceptable salts, hydrates, polymorphs, or solvates thereof is configured for immediate release, extended release, delayed release, or any combination thereof. In some embodiments, the therapeutically effective amount of carbidopa is from about 0.001 mg to about 1,000 mg, or from about 0.001 mg to about 700 mg. In some embodiments, the therapeutically effective amount of carbidopa is about 30 mg, about 40 mg, about 50 mg, about 60 mg, about 70 mg, about 71.25 mg, about 80 mg, about 108.75 mg, about 146.25 mg, 183.75 mg, about 245 mg, about 245 mg, about 306.25 mg, about 367.5 mg, about 428.75 mg, about 490 mg, about 551.25 mg, or about 612.5 mg.

In some embodiments, the at least one additional therapeutic agent is an anticonvulsant. In some embodiments, anticonvulsants for use herein may include, but are not limited, to levetiracitam (Keppra), AMPA receptor antagonists, barbiturate anticonvulsants, benzodiazepine anticonvulsants, carbamate anticonvulsants, carbonic anhydrase inhibitor anticonvulsants, dibenzazepine anticonvulsants, fatty acid derivative anticonvulsants, gamma-aminobutyric acid analogs, gamma-aminobutyric acid reuptake inhibitors, hydantoin anticonvulsants, miscellaneous anticonvulsants, neuronal potassium channel openers, oxazolidinedione anticonvulsants, pyrrolidine anticonvulsants, succinimide anticonvulsants, triazine anticonvulsants or combinations thereof. In some embodiments, the anticonvulsant is administered to a subject in need thereof in a therapeutically effective amount. In some embodiments, the anticonvulsant or pharmaceutically acceptable salts, hydrates or solvates thereof is administered to a subject in need thereof in an amount that is considered to sub therapeutic.

In some embodiments, the at least one additional therapeutic agent is a monoclonal antibody. In some embodiments, the second therapeutic agent is a human monoclonal antibody. In some embodiments, the second therapeutic agent is a humanized monoclonal antibody. In some embodiments the monoclonal antibody targets beta amyloid. In some embodiments the beta amyloid may comprise aggregated beta amyloid such as but not limited to soluble oligomers, insoluble fibrils deposited into amyloid plaque, or a combination thereof. In some embodiments, the monoclonal antibody is Aducanumab (BIIB037), Gantenerumab, Bapineuzumab, Crenezumab, Ponezumab, Solanezumab, SAR228810, MEDI1814, BAN2401, or any combination thereof. In some embodiments, the monoclonal antibody targets alpha-synuclein. In some embodiments, the monoclonal antibody targeting alpha-synuclein is RG-7935, Posiphen, Affitope PD03A, Affitope PD01A, or any combination thereof.

In some embodiments, the at least one additional therapeutic agent is a BACE enzyme inhibitor. In some embodiments, the BACE enzyme inhibitor is CTS-21166, MK-8931, AZD3293, LY3314814, BI 1181181, LY2886721, E2609, RG7129, JNJ-5486911, TAK-070, or any combination thereof.

In some embodiments, the at least one additional therapeutic agent is a RAGE inhibitor. In some embodiments, the RAGE inhibitor is TTP488 (Azeliragon), TTP4000, FPS-ZM1, or any combination thereof.

In some embodiments, the at least one additional therapeutic agent is an antibody targeting Tau. In some embodiments, the antibody targeting Tau is AADVAC-1, AADVAC-2, ACI-35, BMS-986168, RG7345, TRx-237-015 (LMTX), AV-1451, AV-680, Posiphen, or any combination thereof.

In some embodiments, the at least one additional therapeutic agent is a α7 nicotinic acetylcholine receptor modulator. In some embodiments, the α7 nicotinic acetylcholine receptor modulator is Encenicline (EVP-6124), ABT-126, ABT 418, RG3487, Varenicline, A-867744, TC-5219, AVL3288, BMS933043, DSP-3748, or any combination thereof.

In some embodiments, the at least one additional therapeutic agent may include one or more treatments for Alzheimer's disease such as Namzaric™, Exelon®, Aricept® (donepezil hydrochloride), Namenda® (memantine hydrochloride), or galantamine hydrobromide. In some embodiments, described compositions and formulations may be administered in combination with one or more treatments for Parkinson's Disease such as ABT-126 (Abbott Laboratories), pozanicline (Abbott Laboratories), MABT-5102A (AC Immune), Affitope AD-01 (AFFiRiS GmbH), Affitope AD-02 (AFFiRiS GmbH), davunetide (Allon Therapeutics Inc), nilvadipine derivative (Archer Pharmaceuticals), Anapsos (ASAC Pharmaceutical International AIE), ASP-2535 (Astellas Pharma Inc), ASP-2905 (Astellas Pharma Inc), 1 1C-AZD-2184 (AstraZeneca pic), 1 1C-AZD-2995 (AstraZeneca pic), 18F-AZD-4694 (AstraZeneca pic), AV-965 (Avera Pharmaceuticals Inc), AVN-101 (Avineuro Pharmaceuticals Inc), immune globulin intravenous (Baxter International Inc), EVP-6124 (Bayer AG), nimodipine (Bayer AG), BMS-708163 (Bristol-Myers Squibb Co), CERE-110 (Ceregene Inc), CLL-502 (CLL Pharma), CAD-106 (Cytos Biotechnology AG), mimopezil ((Debiopharm SA), DCB-AD1 (Development Centre for Biotechnology), EGb-761 ((Dr Willmar Schwabe GmbH & Co), E-2012 (Eisai Co Ltd), ACC-001 (Elan Corp pic), bapineuzumab (Elan Corp pic), ELND-006 (Elan Pharmaceuticals Inc), atomoxetine (Eli Lilly & Co), LY-2811376 (Eli Lilly & Co), LY-451395 (Eli Lilly & Co), m266 (Eli Lilly & Co), semagacestat (Eli Lilly & Co), solanezumab (Eli Lilly & Co), AZD-103 (Ellipsis Neurotherapeutics Inc), FGLL (ENKAM Pharmaceuticals A/S), EHT-0202 (ExonHit Therapeutics SA), celecoxib (GD Searle & Co), GSK-933776A (GlaxoSmithKline pic), rosiglitazone XR (GlaxoSmithKline pic), SB-742457 (GlaxoSmithKline pic), R-1578 (Hoffmann-La Roche AG), HF-0220 (Hunter-Fleming Ltd), oxiracetam (ISF Societa Per Azioni), KD-501 (Kwang Dong Pharmaceutical Co Ltd), NGX-267 (Life Science Research Israel), huperzine A (Mayo Foundation), Dimebon (Medivation Inc), MEM-1414 (Memory Pharmaceuticals Corp), MEM-3454 (Memory Pharmaceuticals Corp), MEM-63908 (Memory Pharmaceuticals Corp), MK-0249 (Merck & Co Inc), MK-0752 (Merck & Co Inc), simvastatin (Merck & Co Inc), V-950 (Merck & Co Inc), memantine (Merz & Co GmbH), neramexane (Merz & Co GmbH), Epadel (Mochida Pharmaceutical Co Ltd), 123I-MNI-330 (Molecular Neuroimaging Lie), gantenerumab (MorphoSys AG), NIC5-15 (Mount Sinai School of Medicine), huperzine A (Neuro-Hitech Inc), OXIGON (New York University), NP-12 (Noscira SA), NP-61 (Noscira SA), rivastigmine (Novartis AG), ECT-AD (NsGene A/S), arundic acid (Ono Pharmaceutical Co Ltd), PF-3084014 (Pfizer Inc), PF-3654746 (Pfizer Inc), RQ-00000009 (Pfizer Inc), PYM-50028 (Phytopharm pic), Gero-46 (PN Gerolymatos SA), PBT-2 (Prana Biotechnology Ltd), PRX-03140 (Predix Pharmaceuticals Inc), Exebryl-1 (ProteoTech Inc), PF-4360365 (Rinat Neuroscience Corp), HuCAL anti-beta amyloid monoclonal antibodies (Roche AG), EVT-302 (Roche Holding AG), nilvadipine (Roskamp Institute), galantamine (Sanochemia Pharmazeutika AG), SAR-110894 (sanofi-aventis), INM-176 (Scigenic & Scigen Harvest), mimopezil (Shanghai Institute of Materia Medica of the Chinese Academy of Sciences), NEBO-178 (Stegram Pharmaceuticals), SUVN-502 (Suven Life Sciences), TAK-065 (Takeda Pharmaceutical), ispronicline (Targacept Inc), rasagiline (Teva Pharmaceutical Industries), T-817MA (Toyama Chemical), PF-4494700 (TransTech Pharma Inc), CX-717 (University of California), 18F-FDDNP (University of California Los Angeles), GTS-21 (University of Florida), 18F-AV-133 (University of Michigan), 18F-AV-45 (University of Michigan), tetrathiomolybdate (University of Michigan), 1231-IMPY (University of Pennsylvania), 18F-AV-1/ZK (University of Pennsylvania), 11C-6-Me-BTA-1 (University of Pittsburgh), 18F-6-OH-BTA-1 (University of Pittsburgh), MCD-386 (University of Toledo), leuprolide acetate implant (Voyager Pharmaceutical Corp), aleplasinin (Wyeth), begacestat (Wyeth), GSI-136 (Wyeth), NSA-789 (Wyeth), SAM-531 (Wyeth), CTS-21166 (Zapaq), and ZSET-1446 (Zenyaku Kogyo).

In some embodiments, the at least one additional therapeutic agent may include one or more agents useful for the treatment of motor neuronal disorders, such as AEOL-10150 (Aeolus Pharmaceuticals Inc), riluzole (Aventis Pharma AG), ALS-08 (Avicena Group Inc), creatine (Avicena Group Inc), arimoclomol (Biorex Research and Development Co), mecobalamin (Eisai Co Ltd), talampanel (Eli Lilly & Co), R-7010 (F Hoffmann-La Roche Ltd), edaravone (Mitsubishi-Tokyo Pharmaceuticals Inc), arundic acid (Ono Pharmaceutical Co Ltd), PYM-50018 (Phytopharm pic), RPI-MN (ReceptoPharm Inc), SB-509 (Sangamo Biosciences Inc), olesoxime (Trophos SA), sodium phenylbutyrate (Ucyclyd Pharma Inc), and R-pramipexole (University of Virginia).

In some embodiments, the compositions described herein may include one or more agents known to modify cholinergic transmission such as M1 muscarinic receptor agonists or allosteric modulators, M2 muscarinic antagonists, acetylcholinesterase inhibitors, nicotinic receptor agonists or allosteric modulators, 5-HT4 receptor partial agonists or 5HT1A receptor antagonists and NMDA receptor antagonists or modulators, glutamate antagonists, GABA-ergic antagonists, H3 antagonists, putative metabolic/mitochondrial modulators, or disease modifying agents such as β or γ-secretase inhibitors, Tau-targeted therapeutics, β-amyloid aggregation inhibitors and β-amyloid immunotherapies, an antidepressants, for example a tricyclic, a MAOI (Monoamine oxidase inhibitor) a SSRI (Selective Serotonin Reuptake Inhibitor), a SNRI (Serotonin and Noradrenaline Reuptake Inhibitor) or a NaSSA (noradrenergeric and specific serotonergic antidepressant). Examples of specific antidepressant compounds include amitriptyline, clomipramine, citalopram, dosulepin, doxepin, fluoxetine, imipramine, lofepramine, mirtazapine, moclobemide, nortriptyline, paroxetine, phenelzine, reboxetine, sertraline, tranylcypromine, trazodone, or venlafaxine. In some embodiments, additional therapeutic agents may include antipsychotic drugs, such as olanzapine, clozapine, risperidone, quetiapine, aripiprazole or paliperiden.

The therapeutic agents in the methods and compositions described herein may be administered simultaneously or sequentially and, when administration is sequential, either may be administered first, second or third. When administration is simultaneous, the combination may be administered either in the same or different pharmaceutical composition. In some embodiments, the ant-cholinergic agent may be administered between 24 and 72 hours prior to administration of the other agents in the composition. For example, glycopyrrolate may be administered to a subject about 24 hours before the administration of 3-phenylsulfonyl-8-piperazinyl-1yl-quinoline and donepezil.

The therapeutic agents in the methods and compositions described herein may be used either as separate formulations or as a single combined formulation. In some embodiments, the therapeutic agents in the methods and compositions described herein may be configured into separate formulations. For example, a therapeutically effective amount of 3-phenylsulfonyl-8-piperazinyl-1yl-quinoline or pharmaceutically acceptable salts, hydrates, solvates, or polymorphs, thereof, may be configured in a first composition, a therapeutically effective amount of rivastigmine may be configured into a second compositions, and a therapeutically effective amount of glycopyrrolate may be configured into a third composition. In some embodiments, the therapeutic agents in the methods and compositions described herein may be combined into a single formulation. For example, therapeutically effective amounts of 3-phenylsulfonyl-8-piperazinyl-1yl-quinoline or pharmaceutically acceptable salts, hydrates, solvates, or polymorphs, thereof, rivastigmine, and glycopyrrolate may be combined into a single composition. In yet other embodiments, the therapeutic agents in the methods and compositions described herein may be configured into multiple separate compositions. For example, a therapeutically effective amount of 3-phenylsulfonyl-8-piperazinyl-1yl-quinoline or pharmaceutically acceptable salts, hydrates, solvates, or polymorphs, thereof, maybe be formulated into a first composition and therapeutically effective amounts of rivastigmine and glycopyrrolate may be formulated into a second formulation. Alternatively, a therapeutically effective amount of 3-phenylsulfonyl-8-piperazinyl-1yl-quinoline or pharmaceutically acceptable salts, hydrates, solvates, or polymorphs, thereof, may be combined with a therapeutically effective amount of rivastigmine into a first composition and a therapeutically effective amount of glycopyrrolate may be configured into a second composition. Alternatively, a therapeutically effective amount of 3-phenylsulfonyl-8-piperazinyl-1yl-quinoline or pharmaceutically acceptable salts, hydrates, solvates, or polymorphs, thereof, may be combined with a therapeutically effective amount of glycopyrrolate into a first composition and a therapeutically effective amount of rivastigmine may be configured into a second composition. When combined in the same formulation, it will be appreciated that the compounds must be stable and compatible with each other and the other components of the formulation.

In some embodiments, the compositions described herein may further comprise at least one pharmaceutically acceptable excipient is selected from the group consisting of microcrystalline cellulose, mannitol, sodium starch glycolate, hydroxypropyl methylcellulose, purified water, magnesium stearate, croscarmellose sodium, a glue, and any combination thereof.

When the compounds of this disclosure are administered in combination therapies with other agents, they may be administered sequentially or concurrently to the patient. Additional therapeutic agents that are normally administered to treat a particular disease or condition may be referred to as “agents appropriate for the disease, or condition, being treated.”

If pharmaceutically acceptable salts of the compounds of this disclosure are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecyl sulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate. Base salts include ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

Also, the basic nitrogen-containing groups can be quaternized with such agents as lower alkyl halides, such as methyl, ethyl, propyl, and butyl chlorides, bromides and iodides; dialkyl sulfates, such as dimethyl, diethyl, dibutyl and diamyl sulfates; long chain halides such as decyl, lauryl, myristyl and stearyl chlorides, bromides and iodides; aralkyl halides, such as benzyl and phenethyl bromides and others. Water or oil-soluble or dispersible products are thereby obtained.

The compounds utilized in the compositions and methods of this disclosure may also be modified by appending appropriate functionalities to enhance selective biological properties. Such modifications are known in the art and include those, which increase biological penetration into a given biological system (e.g., blood, lymphatic system, or central nervous system), increase oral availability, increase solubility to allow administration by injection, alter metabolism and/or alter rate of excretion.

According to a preferred embodiment, the compositions of this disclosure are formulated for pharmaceutical administration to a subject or patient, e.g., a mammal, preferably a human being. Such pharmaceutical compositions are used to ameliorate, treat or prevent any of the diseases described herein including but not limited to neurodegenerative diseases in a subject.

Agents of the disclosure are often administered as pharmaceutical compositions comprising an active therapeutic agent, i.e., and a variety of other pharmaceutically acceptable components. See Remington's Pharmaceutical Sciences (19th Edition (Mack Publishing Company, 1995)). The preferred form depends on the intended mode of administration and therapeutic application. The compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In some embodiments, the present disclosure provides pharmaceutically acceptable compositions comprising a therapeutically effective amount of one or more of a described compound, formulated together with one or more pharmaceutically acceptable excipients including but not limited to, carriers (additives) and/or diluents for use in treating the diseases described herein, including, but not limited to a neurodegenerative disease. While it is possible for a described compound to be administered alone, it is preferable to administer a described compound as a pharmaceutical formulation (composition) as described herein. Described compounds may be formulated for administration in any convenient way for use in human or veterinary medicine, by analogy with other pharmaceuticals.

As described in detail, pharmaceutical compositions of the present disclosure may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or delayed-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

In some embodiments, the compositions described herein can be configured as overcoated tablet formulations. In some embodiments, the compositions described herein can be configured as an encased product coated edge-to-edge tablet formulations. In some embodiments, a flat-oval edge-to-edge formulation might also be obtained from a hard-gelatin or HPMC capsule manufactured using a flattened mold rather than a circular mold. In some embodiments a “flattened” capsule would be a more desirable alternative to the standard circular capsule.

Pharmaceutically acceptable salts of compounds described herein include conventional nontoxic salts or quaternary ammonium salts of a compound, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenyl acetic, glutamic, benzoic, salicyclic, sulfanilic, 2-acetoxybenzoic, fumaric, toluenesulfonic, methanesulfonic, ethane disulfonic, oxalic, isothionic, and the like. In other cases, described compounds may contain one or more acidic functional groups and, thus, are capable of forming pharmaceutically acceptable salts with pharmaceutically acceptable bases. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations for use in accordance with the present disclosure include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient, which can be combined with a carrier material to produce a single dosage form will vary depending upon the host being treated, and the particular mode of administration. The amount of active ingredient that can be combined with a carrier material to produce a single dosage form will generally be that amount of the compound, which produces a therapeutic effect. Generally, this amount will range from about 1% to about 99% of active ingredient, preferably from about 5% to about 70%, most preferably from about 10% to about 30%.

In certain embodiments, a formulation as described herein comprises an excipient selected from the group consisting of cyclodextrins, liposomes, micelle forming agents, e.g., bile acids, and polymeric carriers, e.g., polyesters and polyanhydrides; and a compound of the present disclosure. In certain embodiments, an aforementioned formulation renders orally bioavailable a described compound of the present disclosure.

The compositions described herein optionally contain inactive carriers and diluents known to one of skill in the art such as, for example microcrystalline cellulose (10-150 mg), mannitol (10-100 mg), sodium starch glycolate (0.001-20 mg, or 1-20 mg), hydroxypropyl methylcellulose (1-20 mg), magnesium stearate (1-10 mg), and purified water.

Methods of preparing formulations or compositions comprising described compounds include a step of bringing into association a compound of the present disclosure with the carrier and, optionally, one or more accessory ingredients (excipients). In general, formulations may be prepared by uniformly and intimately bringing into association a compound of the present disclosure with liquid carriers, or finely divided solid carriers, or both, and then, if necessary, shaping the product.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as those described in Pharmacopeia Helvetica, or a similar alcohol. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In some cases, in order to prolong the effect of a drug, it may be desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the described compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

The pharmaceutical compositions of this disclosure may be orally administered in any orally acceptable dosage form including, but not limited to, capsules, tablets, and aqueous suspensions and solutions. In the case of tablets for oral use, carriers, which are commonly used include but are not limited to lactose and cellulose (carboxymethylcellulose). Lubricating agents, such as magnesium stearate, are also typically added. For oral administration in a capsule form, useful diluents include but are not limited to lactose and cellulose (carboxymethylcellulose). When aqueous suspensions and solutions and propylene glycol are administered orally, the active ingredient is combined with emulsifying and suspending agents. If desired, certain sweetening and/or flavoring and/or coloring agents may be added.

Formulations described herein suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. Compounds described herein may also be administered as a bolus, electuary or paste.

In solid dosage forms for oral administration (capsules, tablets, pills, dragees, powders, granules and the like), an active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

Tablets may be made by compression or molding, optionally with one or more accessory ingredients (excipients). Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent. If a solid carrier is used, the preparation can be in tablet form, placed in a hard gelatin capsule in powder or pellet form, or in the form of a troche or lozenge. The amount of solid carrier will vary, e.g., from about 0.01 to 800 mg, preferably about 0.01 mg to 400 mg, about or 3 mg to about 400 mg. When a liquid carrier is used, the preparation can be, e.g., in the form of a syrup, emulsion, soft gelatin capsule, sterile injectable liquid such as an ampule or nonaqueous liquid suspension. Where the composition is in the form of a capsule, any routine encapsulation is suitable, for example, using the aforementioned carriers in a hard gelatin capsule shell.

Tablets and other solid dosage forms, such as dragees, capsules, pills and granules, may optionally be scored or prepared with coatings and shells, such as enteric coatings and other coatings well known in the pharmaceutical-formulating art. They may alternatively or additionally be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, oral compositions can also include adjuvants such as wetting agents, emulsifying and suspending agents, sweetening, flavoring, coloring, perfuming and preservative agents.

Suspensions, in addition to active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The pharmaceutical compositions of this disclosure may also be administered in the form of suppositories for rectal administration. These compositions can be prepared by mixing a compound of this disclosure with a suitable non-irritating excipient, which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this disclosure is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this disclosure include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this disclosure may also be topically applied to the lower intestinal tract by rectal suppository formulation or in a suitable enema formulation. Topically-administered transdermal patches are also included in this disclosure. Transdermal patches have the added advantage of providing controlled delivery of a compound of the present disclosure to the body. Dissolving or dispersing the compound in the proper medium can make such dosage forms. Absorption enhancers can also be used to increase the flux of the compound across the skin. Either providing a rate controlling membrane or dispersing the compound in a polymer matrix or gel can control the rate of such flux.

The pharmaceutical compositions of this disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

Examples of suitable aqueous and nonaqueous carriers, which may be employed in the pharmaceutical compositions of the disclosure, include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Such compositions may also contain adjuvants such as preservatives, wetting agents, emulsifying agents and dispersing agents. Inclusion of one or more antibacterial and/or antifungal agents, for example, paraben, chlorobutanol, phenol sorbic acid, and the like, may be desirable in certain embodiments. It may alternatively or additionally be desirable to include isotonic agents, such as sugars, sodium chloride, and the like into the compositions. In addition, prolonged absorption of the injectable pharmaceutical form may be brought about by the inclusion of agents, which delay absorption such as aluminum monostearate and gelatin.

In certain embodiments, a described compound or pharmaceutical preparation is administered orally. In other embodiments, a described compound or pharmaceutical preparation is administered intravenously. Alternative routes of administration include sublingual, intramuscular, and transdermal administrations.

When compounds described herein are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

Preparations described herein may be given orally, parenterally, topically, or rectally. They are of course given in forms suitable for the relevant administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

Such compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, compounds described herein which may be used in a suitable hydrated form, and/or the pharmaceutical compositions of the present disclosure, are formulated into pharmaceutically-acceptable dosage forms by conventional methods known to those of skill in the art.

Actual dosage levels of the active ingredients in the pharmaceutical compositions of the disclosure may be varied so as to obtain an amount of the active ingredient that is effective to achieve the desired therapeutic response for a particular patient, composition, and mode of administration, without being toxic to the patient.

The pharmaceutical compositions described herein may be prepared by admixture, suitably at ambient temperature and atmospheric pressure, and is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents, fillers, tableting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavorings or colorants.

For parenteral administration, fluid unit dosage forms are prepared utilizing a compound and a sterile vehicle. The compound, depending on the vehicle and concentration used, can be either suspended or dissolved in the vehicle. In preparing solutions, the compound can be dissolved for injection and filter sterilized before filling into a suitable vial or ampoule and sealing. Advantageously, adjuvants such as a local anesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilized by exposure to ethylene oxide before suspension in a sterile vehicle. Advantageously, a surfactant or wetting agent is included in the composition to facilitate uniform distribution of the compound.

The compositions described herein, used in the treatment of a neurodegenerative disease will vary in the usual way with the seriousness of the disorders, the weight of the sufferer, and other similar factors. However, as a general guide, such unit doses will preferably be administered once a day, although administration more than once a day may be required; and such therapy may extend for a number of weeks or months.

The composition may contain from 0.1% to 99% by weight, preferably from 10 to 60% by weight, of the active material, depending on the method of administration.

Compositions may, if desired, be presented in a pack or dispenser device which may contain one or more unit dosage forms containing the active ingredients. The pack may, for example, comprise metal or plastic foil, such as a blister pack. Where the compounds are intended for administration as two separate compositions these may be presented, for example, in the form of a twin pack.

Pharmaceutical compositions may also be prescribed to the patient in “patient packs” containing the whole course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in traditional prescriptions. The inclusion of a package insert has been shown to improve patient compliance with the physician's instructions.

It will be understood that the administration of the combination by means of a single patient pack, or patient packs of each composition, including a package insert directing the patient to the correct use of the combination is a desirable additional embodiment. Some embodiments are directed to a patient pack comprising at least one active ingredient, of the combination and an information insert containing directions on the use of the combination. Some embodiments are directed to a double pack comprising in association for separate administration of a therapeutically effective amount of 3-phenylsulfonyl-8-piperazinyl-1yl-quinoline or pharmaceutically acceptable salts, hydrates, polymorphs, or solvates thereof and a therapeutically effective amount of at least one additional therapeutic agent useful for treating a neurodegenerative disease.

The dose when using the compounds of the present disclosure can vary within wide limits, and as is customary and is known to the physician, it is to be tailored to the individual conditions in each individual case. It depends, for example, on the nature and severity of the illness to be treated, on the condition of the patient, on the compound employed or on whether an acute or chronic disease state is treated or prophylaxis is conducted or on whether further active compounds are administered in addition to the compounds of the present disclosure. Representative doses of the present disclosure include, but are not limited to, about 0.001 mg to about 5,000 mg. Multiple doses may be administered during the day, especially when relatively large amounts are deemed to be needed, for example 2, 3, or 4, doses. Depending on the individual and as deemed appropriate from the patient's physician or care-giver it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated or prophylaxis is conducted or on whether further active compounds are administered in addition to the compounds of the present disclosure and as part of a drug combination. The dosage regimen for treating a disease condition with the compounds and/or compositions of this disclosure is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods of this disclosure.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3, or 4, part administrations. If appropriate, depending on individual behavior, it may be necessary to deviate upward or downward from the daily dose indicated.

Other solid dosage forms, such as dragees, capsules, pills and granules, may optionally be scored or prepared with coatings and shells, such as enteric coatings and other coatings well known in the pharmaceutical-formulating art. They may alternatively or additionally be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

### Methods

Some embodiments are directed to methods of treating a neurodegenerative disease comprising administering any one of the compositions described herein. In some embodiments, the neurodegenerative disease is selected from the group consisting of acute delirium, delirium, Pick's disease, Fronto-temporal dementia, Progressive Supranuclear Palsy Alzheimer's disease (including mild or early-stage Alzheimer's disease, mild to moderate Alzheimer's disease, moderate or mid-stage Alzheimer's disease, moderate to severe Alzheimer's disease, moderately severe Alzheimer's disease, severe Alzheimer's disease, Alzheimer's disease with Lewy bodies, (AD)), Parkinson's disease (including Parkinson's disease chemically induced by exposure to environmental agents such as pesticides, insecticides, or herbicides and/or metals such as manganese, aluminum, cadmium, copper, or zinc, SNCA gene-linked Parkinson's disease, sporadic or idiopathic Parkinson's disease, or Parkin- or LRRK2-linked Parkinson's disease (PD)), autosomal-dominant Parkinson's disease, Diffuse Lewy Body Disease (DLBD) also known as Dementia with Lewy Bodies (DLB), Pure Autonomic Failure, Lewy body dysphagia, Incidental LBD, Inherited LBD (e.g., mutations of the alpha-synuclein gene, PARK3 and PARK4), multiple system atrophy (including Olivopontocerebellar Atrophy, Striatonigral Degeneration, Shy-Drager Syndrome (MSA)), combined Alzheimer's and Parkinson disease and/or MSA, Huntington's disease, synucleinopathies, disorders or conditions characterized by the presence of Lewy bodies, multiple sclerosis, Amyotrophic lateral sclerosis (ALS) dementia (including vascular dementia, Lewy body dementia, Parkinson's dementia, frontotemporal dementia), Down syndrome, Psychosis (including agitation caused by a neurodegenerative disease or associated with dopaminergic therapy such as but not limited to Parkinson's disease psychosis, Alzheimer's disease psychosis, Lewy body dementia psychosis), dyskinesia (including agitation caused by a neurodegenerative disease or associated with dopaminergic therapy), agitation (including agitation caused by a neurodegenerative disease or associated with dopaminergic therapy), conditions associated with dopaminergic therapy (including dystonia, myoclonus, or tremor), synucleinopathies, diseases, disorders or conditions associated with abnormal expression, stability, activities and/or cellular processing of α-synuclein, diseases, disorders or conditions characterized by the presence of Lewy bodies, and combinations thereof.

Some embodiments herein are directed to methods for treating a neurodegenerative disease comprising administering to a patient a therapeutically effective amount of a 5-HT₆ receptor antagonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises therapeutically effective amount of 3-phenylsulfonyl-8-piperazinyl-1yl-quinoline or pharmaceutically acceptable salts, hydrates or solvates thereof a therapeutically effective amount of donepezil or pharmaceutically acceptable salts, hydrates or solvates thereof a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of donepezil or pharmaceutically acceptable salts, hydrates or solvates thereof a therapeutically effective amount of solifenacin or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof a therapeutically effective amount of rivastigmine or pharmaceutically acceptable salts, hydrates or solvates thereof a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient.

Some embodiments are directed to methods for treating a neurodegenerative disease comprising administering to a patient a therapeutically effective amount of a 5-HT_{2A} inverse agonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient In some embodiments, the method is suitable for oral administration. In some embodiments, the method is suitable for delivery through other routes of administration, including transdermal delivery via patch and/or intranasal delivery.

Some embodiments are directed to methods for treating a neurodegenerative disease comprising administering to a patient a therapeutically effective amount of a NMDA receptor antagonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of memantine or pharmaceutically acceptable salts, hydrates or solvates thereof; a therapeutically effective amount of an donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments herein are directed to methods for treating a neurodegenerative disease comprising administering to a patient a therapeutically effective amount of a 5-HT₆ receptor antagonist; a 5-HT_{2A} inverse agonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments herein are directed to methods for treating a neurodegenerative disease comprising administering to a patient a therapeutically effective amount of a 5-HT₆ receptor antagonist; a NMDA receptor antagonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments herein are directed to methods for treating a neurodegenerative disease comprising administering to a patient a therapeutically effective amount of a 5-HT_{2A} inverse agonist; a NMDA receptor antagonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments are directed to methods for treating a neurodegenerative disease comprising administering to a patient a therapeutically effective amount of a 5-HT₆ receptor antagonist; a therapeutically effective amount of a 5-HT_{2A} inverse agonist; a therapeutically effective amount of a NMDA receptor antagonist; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments are directed to methods for treating a neurodegenerative disease comprising administering to a patient a therapeutically effective amount of a 5-HT₆ receptor antagonist; a therapeutically effective amount of a 5-HT_{2A} inverse agonist; a therapeutically effective amount of a NMDA receptor antagonist; a therapeutically effective amount of an acetylcholinesterase inhibitor; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of donepezil or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of rivastigmine or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments are directed to methods for treating a neurodegenerative disease comprising administering to a patient a therapeutically effective amount of a 5-HT₆ receptor antagonist; a therapeutically effective amount of a 5-HT_{2A} inverse agonist; a therapeutically effective amount of a NMDA receptor antagonist; a therapeutically effective amount of an anti-cholinergic agent; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments herein are directed to methods for treating a neurodegenerative disease comprising administering to a patient a therapeutically effective amount of a 5-HT₆ receptor antagonist; a 5-HT_{2A} inverse agonist; a NMDA receptor antagonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

In some embodiments, the therapeutically effective amount of donepezil is administered to the patient orally, transdermally, intravenously, intranasally or parenterally. In some embodiments, the therapeutically effective amount of rivastigmine is administered to the patient orally, transdermally, intravenously, intranasally or parenterally. In some embodiments, a therapeutically effective amount of trospium is administered to the patient either orally, transdermally, intravenously, intranasally or parenterally. In some embodiments, a therapeutically effective amount of glycopyrrolate is administered to the patient either orally, transdermally, intravenously, intranasally or parenterally. In some embodiments, a therapeutically effective amount of solifenacin is administered to the patient either orally, transdermally, intravenously, intranasally or parenterally.

Some embodiments are directed to methods for enhancing the standard of care in a subject being treated for a neurodegenerative disease comprising administering to a patient any of the composition described herein. In some embodiments, enhancing the standard of care includes enhancing the efficacy and enhancing safety of the standard of care or a combination thereof.

Some embodiments herein are directed to methods for enhancing the standard of care in a subject being treated for a neurodegenerative disease comprising administering to a patient a therapeutically effective amount of a 5-HT₆ receptor antagonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises therapeutically effective amount of 3-phenylsulfonyl-8-piperazinyl-1yl-quinoline or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient.

Some embodiments are directed to methods for enhancing the standard of care in a subject being treated for a neurodegenerative disease comprising administering to a patient a therapeutically effective amount of a 5-HT_{2A} inverse agonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient In some embodiments, the method is suitable for oral administration. In some embodiments, the method is suitable for delivery through other routes of administration, including transdermal delivery via patch and/or intranasal delivery.

Some embodiments are directed to methods for enhancing the standard of care in a subject being treated for a neurodegenerative disease comprising administering to a patient a therapeutically effective amount of a NMDA receptor antagonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of memantine or pharmaceutically acceptable salts, hydrates or solvates thereof; a therapeutically effective amount of an donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments herein are directed to methods for enhancing the standard of care in a subject being treated for a neurodegenerative disease comprising administering to a patient a therapeutically effective amount of a 5-HT₆ receptor antagonist; a 5-HT_{2A} inverse agonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments herein are directed to methods for enhancing the standard of care in a subject being treated for a neurodegenerative disease comprising administering to a patient a therapeutically effective amount of a 5-HT₆ receptor antagonist; a NMDA receptor antagonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments herein are directed to methods for enhancing the standard of care in a subject being treated for a neurodegenerative disease comprising administering to a patient a therapeutically effective amount of a 5-HT_{2A} inverse agonist; a NMDA receptor antagonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments are directed to methods for enhancing the standard of care in a subject being treated for a neurodegenerative disease comprising administering to a patient a therapeutically effective amount of a 5-HT₆ receptor antagonist; a therapeutically effective amount of a 5-HT_{2A} inverse agonist; a therapeutically effective amount of a NMDA receptor antagonist; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments are directed to methods for enhancing the standard of care in a subject being treated for a neurodegenerative disease comprising administering to a patient a therapeutically effective amount of a 5-HT₆ receptor antagonist; a therapeutically effective amount of a 5-HT_{2A} inverse agonist; a therapeutically effective amount of a NMDA receptor antagonist; a therapeutically effective amount of an acetylcholinesterase inhibitor; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of donepezil or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of rivastigmine or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments are directed to methods for enhancing the standard of care in a subject being treated for a neurodegenerative disease comprising administering to a patient a therapeutically effective amount of a 5-HT₆ receptor antagonist; a therapeutically effective amount of a 5-HT_{2A} inverse agonist; a therapeutically effective amount of a NMDA receptor antagonist; a therapeutically effective amount of an anti-cholinergic agent; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments herein are directed to methods for enhancing the standard of care in a subject being treated for a neurodegenerative disease comprising administering to a patient a therapeutically effective amount of a 5-HT₆ receptor antagonist; a 5-HT_{2A} inverse agonist; a NMDA receptor antagonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

In some embodiments, the therapeutically effective amount of donepezil is administered to the patient orally, transdermally, intravenously, intranasally or parenterally. In some embodiments, the therapeutically effective amount of rivastigmine is administered to the patient orally, transdermally, intravenously, intranasally or parenterally. In some embodiments, a therapeutically effective amount of trospium is administered to the patient either orally, transdermally, intravenously, intranasally or parenterally. In some embodiments, a therapeutically effective amount of glycopyrrolate is administered to the patient either orally, transdermally, intravenously, intranasally or parenterally. In some embodiments, a therapeutically effective amount of solifenacin is administered to the patient either orally, transdermally, intravenously, intranasally or parenterally.

Some embodiments are directed to methods for enhancing the efficacy of a treatment without causing cholinergic toxicity or cholinergic side effects in a subject being treated for a neurodegenerative disease comprising administering to a patient any of the compositions described herein.

Some embodiments herein are directed to methods for enhancing the efficacy of a treatment without causing cholinergic toxicity or cholinergic side effects in a subject being treated for a neurodegenerative disease comprising administering to a patient a therapeutically effective amount of a 5-HT₆ receptor antagonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises therapeutically effective amount of 3-phenylsulfonyl-8-piperazinyl-1yl-quinoline or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient.

Some embodiments are directed to methods for enhancing the efficacy of a treatment without causing cholinergic toxicity or cholinergic side effects in a subject being treated for a neurodegenerative disease comprising administering to a patient a therapeutically effective amount of a 5-HT_{2A} inverse agonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient In some embodiments, the method is suitable for oral administration. In some embodiments, the method is suitable for delivery through other routes of administration, including transdermal delivery via patch and/or intranasal delivery.

Some embodiments are directed to methods for enhancing the efficacy of a treatment without causing cholinergic toxicity or cholinergic side effects in a subject being treated for a neurodegenerative disease comprising administering to a patient a therapeutically effective amount of a NMDA receptor antagonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of memantine or pharmaceutically acceptable salts, hydrates or solvates thereof; a therapeutically effective amount of an donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments herein are directed to methods for enhancing the efficacy of a treatment without causing cholinergic toxicity or cholinergic side effects in a subject being treated for a neurodegenerative disease comprising administering to a patient a therapeutically effective amount of a 5-HT₆ receptor antagonist; a 5-HT_{2A} inverse agonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments herein are directed to methods for enhancing the efficacy of a treatment without causing cholinergic toxicity or cholinergic side effects in a subject being treated for a neurodegenerative disease comprising administering to a patient a therapeutically effective amount of a 5-HT₆ receptor antagonist; a NMDA receptor antagonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments herein are directed to methods for enhancing the efficacy of a treatment without causing cholinergic toxicity or cholinergic side effects in a subject being treated for a neurodegenerative disease comprising administering to a patient a therapeutically effective amount of a 5-HT_{2A} inverse agonist; a NMDA receptor antagonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments are directed to methods for enhancing the efficacy of a treatment without causing cholinergic toxicity or cholinergic side effects in a subject being treated for a neurodegenerative disease comprising administering to a patient a therapeutically effective amount of a 5-HT₆ receptor antagonist; a therapeutically effective amount of a 5-HT_{2A} inverse agonist; a therapeutically effective amount of a NMDA receptor antagonist; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments are directed to methods for enhancing the efficacy of a treatment without causing cholinergic toxicity or cholinergic side effects in a subject being treated for a neurodegenerative disease comprising administering to a patient a therapeutically effective amount of a 5-HT₆ receptor antagonist; a therapeutically effective amount of a 5-HT_{2A} inverse agonist; a therapeutically effective amount of a NMDA receptor antagonist; a therapeutically effective amount of an acetylcholinesterase inhibitor; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of donepezil or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of rivastigmine or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments are directed to methods for enhancing the efficacy of a treatment without causing cholinergic toxicity or cholinergic side effects in a subject being treated for a neurodegenerative disease comprising administering to a patient a therapeutically effective amount of a 5-HT₆ receptor antagonist; a therapeutically effective amount of a 5-HT_{2A} inverse agonist; a therapeutically effective amount of a NMDA receptor antagonist; a therapeutically effective amount of an anti-cholinergic agent; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments herein are directed to methods for enhancing the efficacy of a treatment without causing cholinergic toxicity or cholinergic side effects in a subject being treated for a neurodegenerative disease comprising administering to a patient a therapeutically effective amount of a 5-HT₆ receptor antagonist; a 5-HT_{2A} inverse agonist; a NMDA receptor antagonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

In some embodiments, the therapeutically effective amount of donepezil is administered to the patient orally, transdermally, intravenously, intranasally or parenterally. In some embodiments, the therapeutically effective amount of rivastigmine is administered to the patient orally, transdermally, intravenously, intranasally or parenterally. In some embodiments, a therapeutically effective amount of trospium is administered to the patient either orally, transdermally, intravenously, intranasally or parenterally. In some embodiments, a therapeutically effective amount of glycopyrrolate is administered to the patient either orally, transdermally, intravenously, intranasally or parenterally. In some embodiments, a therapeutically effective amount of solifenacin is administered to the patient either orally, transdermally, intravenously, intranasally or parenterally.

Some embodiments are directed to methods for reducing side effects in a subject being treated for a neurodegenerative disease comprising administering to a patient a composition described herein. Some embodiments are directed to methods of reducing side effects including side effects associated with cholinesterase inhibitors, in a subject being treated for a neurodegenerative disease. In some embodiments, the side effects include, but are not limited to depression, aggression, abdominal pain, anxiety, drowsiness, tremor, fatigue, dizziness, dyspepsia, headache, weakness, diaphoresis, malaise, anorexia, and flatulence, dry mouth, falls, abnormal vision, delirium, diarrhea, constipation, indigestion, loss of appetite, loss of strength, muscle cramps, nausea, trouble sleeping, unusual tiredness or weakness, vomiting, abnormal dreams, constipation, dizziness, drowsiness, fainting, frequent urination, headache, joint pain, stiffness, or swelling, mental depression, pain, unusual bleeding or bruising, weight loss, black tarry stools, bloating, bloody or cloudy urine, blurred vision, burning, prickling, or tingling sensations, cataract, chills, clumsiness or unsteadiness, confusion, cough, decreased urination, difficult or painful urination, dryness of mouth, eye irritation, fever, flushing of skin, frequent urge to urinate, high or low blood pressure, hives, hot flashes, increase in sexual desire or performance, increased heart rate and breathing, increased sweating, increased urge to urinate during the night, irregular heartbeat, itching, loss of bladder control, loss of bowel control, mood or mental changes, including abnormal crying, aggression, agitation, delusions, irritability, nervousness, or restlessness, nasal congestion, pain in chest, upper stomach, or throat, problems with speech, runny nose, severe thirst, shortness of breath, sneezing, sore throat, sunken eyes, tightness in chest, tremor, troubled breathing, wheezing, wrinkled skin, back, leg, or stomach pains, bleeding gums, chest pain or discomfort, coma, convulsions, dark urine, difficulty breathing, fast or irregular heartbeat, fatigue, general body swelling, general tiredness and weakness, high fever, increased thirst, indigestion, light-colored stools, muscle pain or cramps, nausea and vomiting, nosebleeds, pains in stomach, side, or abdomen, possibly radiating to the back, pale skin, rash, seeing, hearing, or feeling things that are not there, seizures, severe muscle stiffness, severe nausea, slow or irregular heartbeat, stomach pain, sweating, swelling of face, ankles, or hands, tiredness, unusually pale skin, upper right abdominal or stomach pain, yellow eyes and skin or any combination thereof.

Some embodiments herein are directed to methods for reducing side effects including side effects associated with cholinesterase inhibitors, in a subject being treated for a neurodegenerative disease comprising administering to a patient a therapeutically effective amount of a 5-HT₆ receptor antagonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises therapeutically effective amount of 3-phenylsulfonyl-8-piperazinyl-1yl-quinoline or pharmaceutically acceptable salts, hydrates or solvates thereof a therapeutically effective amount of donepezil or pharmaceutically acceptable salts, hydrates or solvates thereof a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of donepezil or pharmaceutically acceptable salts, hydrates or solvates thereof a therapeutically effective amount of solifenacin or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient.

Some embodiments are directed to methods for reducing side effects including side effects associated with cholinesterase inhibitors, in a subject being treated for a neurodegenerative disease comprising administering to a patient a therapeutically effective amount of a 5-HT_{2A} inverse agonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient In some embodiments, the method is suitable for oral administration. In some embodiments, the method is suitable for delivery through other routes of administration, including transdermal delivery via patch and/or intranasal delivery.

Some embodiments are directed to methods for reducing side effects including side effects associated with cholinesterase inhibitors, in a subject being treated for a neurodegenerative disease comprising administering to a patient a therapeutically effective amount of a NMDA receptor antagonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of memantine or pharmaceutically acceptable salts, hydrates or solvates thereof; a therapeutically effective amount of an donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments herein are directed to methods for reducing side effects including side effects associated with cholinesterase inhibitors, in a subject being treated for a neurodegenerative disease comprising administering to a patient a therapeutically effective amount of a 5-HT₆ receptor antagonist; a 5-HT_{2A} inverse agonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments herein are directed to methods for reducing side effects including side effects associated with cholinesterase inhibitors, in a subject being treated for a neurodegenerative disease comprising administering to a patient a therapeutically effective amount of a 5-HT₆ receptor antagonist; a NMDA receptor antagonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments herein are directed to methods for reducing side effects including side effects associated with cholinesterase inhibitors, in a subject being treated for a neurodegenerative disease comprising administering to a patient a therapeutically effective amount of a 5-HT_{2A} inverse agonist; a NMDA receptor antagonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; memantine or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments are directed to methods for reducing side effects including side effects associated with cholinesterase inhibitors, in a subject being treated for a neurodegenerative disease comprising administering to a patient a therapeutically effective amount of a 5-HT₆ receptor antagonist; a therapeutically effective amount of a 5-HT_{2A} inverse agonist; a therapeutically effective amount of a NMDA receptor antagonist; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments are directed to methods for reducing side effects including side effects associated with cholinesterase inhibitors, in a subject being treated for a neurodegenerative disease comprising administering to a patient a therapeutically effective amount of a 5-HT₆ receptor antagonist; a therapeutically effective amount of a 5-HT_{2A} inverse agonist; a therapeutically effective amount of a NMDA receptor antagonist; a therapeutically effective amount of an acetylcholinesterase inhibitor; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of donepezil or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of rivastigmine or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments are directed to methods for reducing side effects including side effects associated with cholinesterase inhibitors, in a subject being treated for a neurodegenerative disease comprising administering to a patient a therapeutically effective amount of a 5-HT₆ receptor antagonist; a therapeutically effective amount of a 5-HT_{2A} inverse agonist; a therapeutically effective amount of a NMDA receptor antagonist; a therapeutically effective amount of an anti-cholinergic agent; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1 yl-quinoline or pharmaceutically acceptable salts, hydrates or solvates thereof; a therapeutically effective amount of 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

Some embodiments herein are directed to methods for reducing side effects including side effects associated with cholinesterase inhibitors, in a subject being treated for a neurodegenerative disease comprising administering to a patient a therapeutically effective amount of a 5-HT₆ receptor antagonist; a 5-HT_{2A} inverse agonist; a NMDA receptor antagonist; a therapeutically effective amount of an acetylcholinesterase inhibitor; a therapeutically effective amount of an anti-cholinergic agent; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of donepezil or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of trospium or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of glycopyrrolate or pharmaceutically acceptable salts, hydrates or solvates thereof; and at least one pharmaceutically acceptable excipient. In some embodiments, the method comprises a therapeutically effective amount of 3-phenylsulfonyl-8-piperazinyl-1yl-quinoline or pharmaceutically acceptable salts, hydrates or solvates thereof; 1-[3-(4-bromo-2-methyl-2H-pyrazol-3-yl)-4-methoxy-phenyl]-3-(2,4-difluoro-phenyl)-urea or pharmaceutically acceptable salts, hydrates or solvates thereof; a therapeutically effective amount of memantine or pharmaceutically acceptable salts, hydrates or solvates thereof; a therapeutically effective amount of rivastigmine or pharmaceutically acceptable salts, hydrates or solvates thereof; a therapeutically effective amount of solifenacin or pharmaceutically acceptable salts, hydrates or solvates thereof; and at least one pharmaceutically acceptable excipient. In some embodiments, the method is suitable for oral administration. In some embodiments, the method is suitable for delivery through other routes of administration, including transdermal delivery via patch and/or intranasal delivery.

In some embodiments, the therapeutically effective amount of donepezil is administered to the patient orally, transdermally, intravenously, intranasally or parenterally. In some embodiments, the therapeutically effective amount of rivastigmine is administered to the patient orally, transdermally, intravenously, intranasally or parenterally. In some embodiments, a therapeutically effective amount of trospium is administered to the patient either orally, transdermally, intravenously, intranasally or parenterally. In some embodiments, a therapeutically effective amount of glycopyrrolate is administered to the patient either orally, transdermally, intravenously, intranasally or parenterally. In some embodiments, a therapeutically effective amount of solifenacin is administered to the patient either orally, transdermally, intravenously, intranasally or parenterally.

In some embodiments, the therapeutically effective amount of 3-phenylsulfonyl-8-piperazinyl-1yl-quinoline or pharmaceutically acceptable salts, hydrates, polymorphs, or solvates thereof is from about 0.001 mg to about 1,000 mg, about 0.001 mg to about 200 mg, about 0.001 mg to about 175 mg, or 0.001 mg to about 70 mg. In some embodiments, the therapeutically effective amount of 3-phenylsulfonyl-8-piperazinyl-1yl-quinoline or pharmaceutically acceptable salts, hydrates, polymorphs, or solvates thereof is about 15 mg, about 35 mg, or about 70 mg. In some embodiments, the therapeutically effective amount of 3-phenylsulfonyl-8-piperazinyl-1yl-quinoline or pharmaceutically acceptable salts, hydrates, polymorphs, or solvates thereof is an amount selected from the group consisting of an amount of 3-phenylsulfonyl-8-piperazinyl-1yl-quinoline that may cause convulsions in a subject to which it is administered; an amount that would be expected to exceed the maximum tolerated dose for the subject to which it is administered; an amount associated with systemic exposures characterized by an AUCₜₐᵤ₋ₛₛ of about 8.2 μg·h/ml, a Cₘₐₓ of about 0.26 μg/ml; or a combination thereof an mount associated with systemic exposures characterized by an AUC, Cₘₐₓ, or combinations thereof, that are about 2 to about 3 times higher than the mean clinical exposure achieved at the proposed clinical dose for monotherapy with 3-phenylsulfonyl-8-piperazinyl-1yl-quinoline (i.e. mean AUCₜₐᵤ₋ₛₛ of about 3.2 μg·h/ml and Cₘₐₓ of about 0.180 μg/ml), an amount associated with a recorded systemic clinical exposure that is greater than the highest recorded systemic clinical exposure (AUC_{0-∞} of about 9.25 μg·h/ml and Cₘₐₓ of about 0.293 μg/ml), an amount of 3-phenylsulfonyl-8-piperazinyl-1yl-quinoline that is greater than about 10 mg/kg/day, an amount of 3-phenylsulfonyl-8-piperazinyl-1yl-quinoline that is greater than 15 mg/day, a dose of 3-phenylsulfonyl-8-piperazinyl-1yl-quinoline that is greater than about 35 mg/day or any combination thereof.

Some embodiments comprise administering between about 0.001 mg/day and about 1,000 mg/day, about 0.001 mg/day and about 30 mg/day, or about 34.5 mg/day to about 92 mg/day of donepezil to the patient. Some embodiments comprise administering about 5 mg/day, 10 mg/day, or 23 mg/day of donepezil to the patient. Some embodiments comprise administering about 23 mg/day of donepezil to the patient. In some embodiments, the therapeutically effective amount of donepezil is about 1.001 to about 1,000 times greater than a recommended maximal dose level approved by the U.S. FDA. In some embodiments, the therapeutically effective amount of donepezil is about 1.5 to about 4 times greater than a recommended maximal dose level approved by the U.S. FDA. For Example, the dose of donepezil may be from about 34.5 mg to about 92 mg.

Some embodiments comprise administering a minimum of about 12 mg/day orally or a minimum of about 9.5 mg/day transdermally of rivastigmine to the patient. Some embodiments comprise administering between about 12 and about 48 mg/day orally or administering between about 19 mg and 54 mg transdermally of rivastigmine to the patient. In some embodiments, the therapeutically effective amount of rivastigmine is about 1.001 to about 1,000 times greater than a recommended maximal dose level approved by the U.S. FDA. In some embodiments, the therapeutically effective amount of rivastigmine is about 1.5 to about 4 times greater than a recommended maximal dose level approved by the U.S. FDA. For Example, the dose of rivastigmine may be from about 18 mg to about 48 mg.

In some embodiments, the therapeutically effective amount of glycopyrrolate is an amount from about 20% to about 600% of the amount of glycopyrrolate that is currently administered for anti-cholinergic therapy. In some embodiments, the therapeutically effective amount of glycopyrrolate is an amount from about 0.2 mg to about 60 mg. Some embodiments comprise administering from about 0.1 to about 0.8 mg/day parenterally, or about 3.0 to about 10.0 mg/day orally of glycopyrrolate to the patient. Some embodiments comprise administering a minimum of about 2-4 mg/day of glycopyrrolate to the patient.

In some embodiments, the therapeutically effective amount of solifenacin is an amount from about 20% to about 600% of the amount of solifenacin that is currently administered for anti-cholinergic therapy. In some embodiments, the therapeutically effective amount of solifenacin is an amount from about 1 mg to about 30 mg.

In some embodiments, the therapeutically effective amount of 3-phenylsulfonyl-8-piperazinyl-1yl-quinoline is administered once a day, twice a day, three times a day, or four times a day. In some embodiments, the therapeutically effective amount of glycopyrrolate is administered once a day, twice a day, three times a day, or four times a day. In some embodiments, the therapeutically effective amount of solifenacin is administered once a day, twice a day, three times a day, or four times a day. In some embodiments, the therapeutically effective amount of donepezil is administered once a day, twice a day, three times a day, or four times a day. In some embodiments, the therapeutically effective amount of rivastigmine is administered once a day, twice a day, three times a day, or four times a day. In some embodiments, the therapeutically effective amount of 1-[3-(4-bromo-2-methyl-2H-pyrazol-3-yl)-4-methoxy-phenyl]-3-(2,4-difluoro-phenyl)-urea is administered once a day, twice a day, three times a day, or four times a day. In some embodiments, the therapeutically effective amount of memantine is administered once a day, twice a day, three times a day, or four times a day. In some embodiments, the therapeutically effective amount of rivastigmine is administered once a day, twice a day, three times a day, or four times a day. In some embodiments, the therapeutically effective amount of tacrine is administered once a day, twice a day, three times a day, or four times a day. In some embodiments, the therapeutically effective amount of galantamine is administered once a day, twice a day, three times a day, or four times a day.

Some embodiments comprise administering trospium. In some embodiments, the quaternary ammonium anti-cholinergic muscarinic receptor antagonist is trospium or pharmaceutically acceptable salts, hydrates or solvates thereof. In some embodiments, the quaternary ammonium anti-cholinergic muscarinic receptor antagonist is trospium chloride. In some embodiments, the therapeutically effective amount of trospium or pharmaceutically acceptable salts, hydrates or solvates thereof is from about 0.1 mg to about 120 mg. In some embodiments, the therapeutically effective amount of trospium or pharmaceutically acceptable salts, hydrates or solvates thereof is about 20 mg, about 40 mg, or about 60 mg. In some embodiments, the therapeutically effective amount of trospium or pharmaceutically acceptable salts, hydrates or solvates thereof is about 20 mg per day, about 40 mg per day, or about 60 mg per day. In some embodiments, the therapeutically effective amount of trospium is an amount from about 20% to about 600% of the amount of trospium or pharmaceutically acceptable salts, hydrates or solvates thereof that is currently administered for anti-cholinergic therapy.

In some embodiments, the various agents described in the compositions and methods described herein are configured as a single subunit, or two or more subunits. Some embodiments further comprise at least one pharmaceutically acceptable excipient. In some embodiments, the at least one pharmaceutical acceptable excipient is configured into the single subunit, or the two or more subunits. In some embodiments, the single subunit comprises a bar, beads, a block, particles, pellets, granules, fibers, globules, powders, a pill, a capsule, a tablet, a caplet, an orally disintegrating tablet, an osmotic controlled-release oral delivery system and any combination thereof. In some embodiments, the tablet is a monolayer tablet, a bilayer tablet, or a multilayer tablet or a combination thereof. In some embodiments, the single subunit further comprises an encapsulation medium. In some embodiments, the encapsulation medium is a capsule, a soft gel cap, a gel cap, a coating, or any combination thereof. In some embodiments, the coating comprises a membrane, a film, a wax, a varnish, a glaze, a polymer coating, a sugar coating, a polysaccharide based coating, an enteric coating, or a combination thereof.

In some embodiments, the various agents described in the compositions and methods described herein are independently configured for immediate release, sustained release, extended release, or any combination thereof.

In some embodiments, the various agents described in the compositions and methods described herein are independently configured for immediate release, sustained release, extended release, or any combination thereof.

In some embodiments, the various agents described in the compositions and methods described herein are independently configured into two or more subunits. In some embodiments, the two or more subunits independently comprise a bar, beads, a block, particles, pellets, granules, fibers, globules, powders, a pill, a capsule, a tablet, a caplet, an orally disintegrating tablet, an osmotic controlled-release oral delivery system and any combination thereof. In some embodiments, the tablet is a monolayer tablet, a bilayer tablet, or a multilayer tablet or a combination thereof.

In some embodiments, the various agents described in the compositions and methods described herein may be combined into an encapsulation medium. In some embodiments, wherein the encapsulation medium is a capsule, a soft gel cap, a gel cap, a coating, or any combination thereof. In some embodiments, the coating comprises a membrane, a film, a wax, a varnish, a glaze, a polymer coating, a sugar coating, a polysaccharide based coating, an enteric coating, or a combination thereof. In some embodiments, the two or more subunits independently comprise a bar, beads, a block, particles, pellets, granules, fibers, globules, powders, a pill, a capsule, a tablet, a caplet, an orally disintegrating tablet, an osmotic controlled-release oral delivery system and any combination thereof. In some embodiments, the tablet is a monolayer tablet, a bilayer tablet, or a multilayer tablet or a combination thereof. In some embodiments, the encapsulation medium is a capsule, a soft gel cap, a gel cap, a coating, or any combination thereof. In some embodiments, the coating comprises a membrane, a film, a wax, a varnish, a glaze, a polymer coating, a sugar coating, a polysaccharide based coating, an enteric coating, or a combination thereof.

In some embodiments, the tablet is a monolayer tablet, a bilayer tablet, or a multilayer tablet or a combination thereof.

In some embodiments, the two or more subunits comprise the therapeutically effective amount of 3-phenylsulfonyl-8-piperazinyl-1yl-quinoline or pharmaceutically acceptable salts, hydrates or solvates thereof configured into a first subunit, and the therapeutically effective amount of at least one additional therapeutic agent useful for the treatment of neurodegenerative disease configured into at least one additional subunit.

In some embodiments, the wherein the compositions described herein are administered in the evening, just prior to retiring. In some embodiments, the compositions described herein are administered to said subject is unchanged or stable during treatment. In some embodiments, compositions described herein are administered daily for a period of time, an extended period of time, for the remainder of the subject's life, for an indefinite period of time, for at least one week, for at least one month or for at least 24 weeks.

In some embodiments, the compositions described herein are administered without a dosage titration. In some embodiments, one or more of the agents in the compositions and methods described herein may be administered using a titration regimen whereby the dose of the agent is gradually increased over time until a therapeutic dosage is reached. In some embodiments, one or more of the agents may be administered at its intended therapeutic dose without titration while another may be titrated up to its therapeutic dosage.

In some embodiments, administration of an anti-cholinergic agent allows for administration of other agents that would normally require a dose to titration to be administered at their full therapeutic dose without the need for a dose titration. For example, the acetylcholinesterase inhibitor donepezil is normally given at an initial dose of 5 mg per day for the first 4 to 6 weeks of treatment after which the dose of drug is increased to 10 mg and then to 23 mg per day. The compositions and methods described herein would permit a subject to receive an initial dose of 10 or 23 mg per day at the onset of treatment without the need for a dose titration thereby maximizing the therapeutic effect of the acetylcholinesterase inhibitor.

In some embodiments, the compositions described herein may cause adverse events in certain subjects. For example, constipation may worsen with anti-cholinergic agents. In some embodiments, this may be counteracted with the common stool softener docusate sodium, 100 mg BID, may be used. In some embodiments, a combination of a stool softener such as docusate sodium, and a laxative such as senna could be used. In some embodiments Doc-Q-Lax (OTC; dosages of 17.2 mg sennosides plus 100 mg docusate) can be given once daily or up to 4 tablets twice daily. In some embodiments, dry mouth may worsen with anti-cholinergic agents, which may lead to poor dentition or choking. In some embodiments, a combination a biotene topical gel (which provides ‘artificial saliva’) can used with the compositions and methods described herein and can additionally be incorporated in the compositions described herein. In some embodiments, nightmares worsen with high-dose rivastigmine in patients with dementia. In some embodiments, this may be due to increased REM sleep duration and REM density. In some embodiments, nelotanserin which decreases time in REM sleep duration may be beneficial to reduce frequencies of nightmares at doses from 10 mg up to 80 mg. In some embodiments, prazosin, an alpha-1 blocker, has been shown to decrease nightmares in patients with PTSD in RCTs, patients who often have fearful nightmares. The content of cholinergic-induced nightmares tend to be vivid and frightening, hence, prazosin from a dose of 1 mg at bedtime to 3 or 10 mg as tolerated may prove beneficial. Prazosin as an alpha-1 blocker is approved as anti-hypertensive. It should be used with caution in patients with autonomic failure or orthostatic hypotension which is common in elderly patients with dementia.

### EXAMPLES

The following examples are illustrative, but not limiting, of the methods and compositions described herein. Other suitable modifications and adaptations of the variety of conditions and parameters normally encountered in therapy and that are obvious to those skilled in the art are within the spirit and scope of the compounds and methods described herein.

### Example 1: A Phase 1 Study to Evaluate the Safety, Tolerability, and Pharmacokinetics of Combinations of Donepezil with Glycopyrrolate or Trospium in Elderly Volunteers

Acetylcholinesterase inhibitors are widely prescribed for the treatment of Alzheimer's disease (AD) and work by blocking the cholinesterase enzyme and preventing the breakdown of acetylcholine. While increasing the concentration of acetylcholine in the brain is beneficial for AD patients and improves cognition and function, increasing acetylcholine in the periphery can cause adverse events including nausea, vomiting, and diarrhea. A potential strategy to attenuate these systemic adverse events while preserving beneficial effects on cognition and function involves the concomitant administration of peripherally active antimuscarinic anticholinergic drugs that do not penetrate the blood/brain barrier (BBB). The RVT-103 program combines donepezil with either glycopyrrolate or trospium, two currently approved quaternary ammonium muscarinic receptor antagonists with low potential to cross the BBB, in an effort to mitigate the peripheral side effects of donepezil. This presentation provides an overview of a Phase 1 study to evaluate the safety, tolerability and pharmacokinetics of combinations of donepezil with either glycopyrrolate or trospium in elderly volunteers.

This study involves three parts:

Part 1: double-blind single dose study in which subjects are randomized to single doses of 10 mg donepezil with either placebo, glycopyrrolate 2 mg, or trospium XR 60 mg.
Part 2: double blind, double dummy repeat dose study in which subjects are randomized to receive either 5 mg donepezil QD+placebo BID, 10 mg donepezil QD+1 mg glycopyrrolate BID, 10 mg donepezil QD+2 mg glycopyrrolate QD, or 10 mg donepezil+60 mg trospium XR QD for 10 days.
Part 3: an open label, repeat dose study in which subjects receive glycopyrrolate 2 mg QD for 3 days to assess concentrations in the cerebrospinal fluid.

Primary endpoints include the incidence of adverse events, laboratory values, and vital signs with secondary endpoints of pharmacokinetic parameters of donepezil, glycopyrrolate, and trospium. This Phase 1 study evaluates the safety, tolerability, and pharmacokinetics of these combinations.

### Example 2: A Phase 1 Study to Evaluate the Safety, Tolerability, and Pharmacokinetics of Combinations of Donepezil with Glycopyrrolate or Trospium in Elderly Volunteers

Background: Cholinesterase inhibitors are widely prescribed for the treatment of AD and are thought to provide therapeutic benefit via inhibition of cholinesterase enzymes, thereby increasing the concentration of acetylcholine (ACh) in the synapse. While increasing the concentration of ACh in the brain results in therapeutic benefit for AD patients, increasing ACh in the periphery can cause adverse effects including nausea, vomiting, and diarrhea.¹ These adverse peripheral effects can limit tolerability and the usefulness of cholinesterase inhibitors for many patients. Glycopyrrolate and trospium are two quaternary ammonium anti-muscarinic agents that have a low propensity to cross the blood/brain barrier. It is hypothesized that, in combination with cholinesterase inhibitors, these agents could 1) facilitate immediate administration of therapeutic doses of cholinesterase inhibitors, and 2) improve tolerability of cholinesterase inhibitors by mitigating peripheral cholinergic side effects associated with their use. This study looked at fixed-dose, non-titrated combinations of donepezil with either glycopyrrolate or trospium in an effort to mitigate the peripheral cholinergic side effects of donepezil.

This proof-of-concept study was designed to characterize the pharmacokinetic profile and tolerability of 5 mg donepezil plus placebo compared to 10 mg donepezil given concomitantly with one of three different fixed-dose regimens of either glycopyrrolate or trospium (A, B, or C). Gastrointestinal tolerability was assessed at steady-sate by Visual Analog Scale (VAS) obtained at baseline and every two hours over an eight hour observation period on Day 10. Additional measures were obtained including standard laboratory values, vital signs, and pharmacokinetic profiles of donepezil, glycopyrrolate, and trospium.

TABLE 3
Subject demographics

| Category | Value (N = 48) |
|---|---|
| Gender | Male (30; 62.5%) |
| | Female (18; 37.5%) |
| Age (Years) | 66.2 (range 55-83) |
| Race | White (45; 93.8%) |
| | Other (3; 6.2%) |
| Weight (Kg) | 74.9 (range 48-107) |
| BMI (Kg/m²) | 25.1 (range 19-30) |

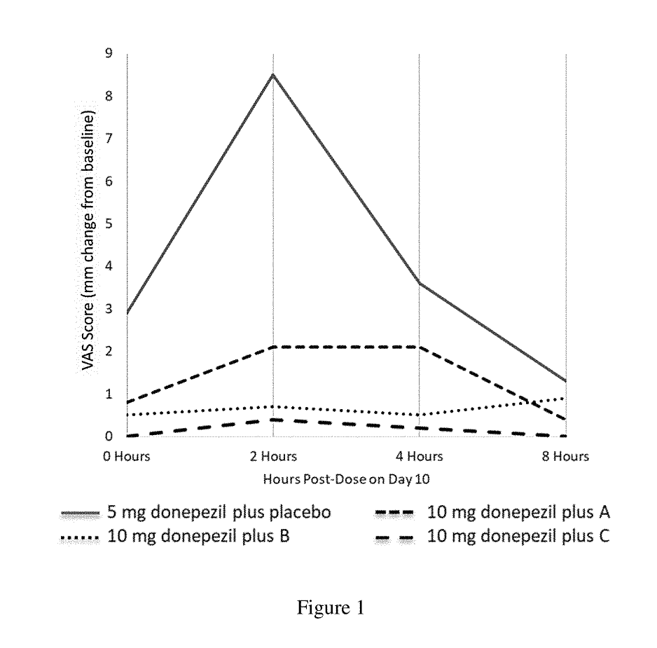

FIG. 1 shows self-reported nausea (VAS score; 0-100 mm). On day 10 (at steady state) nausea was assessed every two hours over an 8 hour observation period after dosing with 5 mg donepezil plus placebo or 10 mg donepezil with one of three different fixed-dose regimens of either glycopyrrolate or trospium (A, B, or C).

The number of subjects completing the study was similar across treatments (one drop out in a 10 mg donepezil plus A/B/C groups). On day 10, at steady state, self-reported nausea using a VAS score in subjects receiving 5 mg donepezil plus placebo, demonstrated a peak mean change from baseline of 8.5 mm. Subjects receiving 10 mg donepezil plus treatment with a regimen containing varying dosages of either glycopyrrolate or trospium (regimen A, B or C on the chart) experienced a 67% to 95% reduction in self-reported nausea relative to the 5 mg donepezil plus placebo arm.

It has been shown that therapeutically relevant doses of donepezil (10 mg QD) can be achieved from initial dosing when administered in combination with a fixed-dose regimen of a quaternary ammonium anti-muscarinic agent. In the study data reported here, at steady state (day 10), the incidence of self-reported nausea measured by VAS score in subjects receiving 10 mg donepezil combined with either glycopyrrolate or trospium was 67% to 95% lower than subjects receiving 5 mg donepezil plus placebo. Concurrent administration of cholinesterase inhibitors with either of the quaternary ammonium anti-muscarinic agents, glycopyrrolate or trospium may 1) facilitate early administration of therapeutic cholinesterase inhibitor doses, and 2) improve tolerability by mitigating peripheral cholinergic side effects associated with cholinesterase inhibitors.

### Example 3: A Phase 1 Study to Evaluate the Safety, Tolerability and Pharmacokinetics of Donepezil with Glycopyrrolate or Trospium in Elderly Volunteers

Acetylcholinesterase inhibitors (AChEIs) are widely prescribed for the treatment of Alzheimer's disease (AD) and are thought to result in therapeutic benefit via inhibition of acetylcholinesterase, thereby increasing the concentration of acetylcholine (ACh) in the synapse. While increasing the concentration of ACh in the brain results in therapeutic benefit for AD patients, increasing ACh in the periphery can cause adverse effects including nausea, vomiting, and diarrhea. These adverse peripheral effects can be medically consequential, limiting the usefulness of AChEIs for many patients. Peripherally active antimuscarinic anticholinergic agents have been theorized as a practical method of reducing these undesired peripheral actions of ACh, which are mediated via peripheral muscarinic receptor activation. Glycopyrrolate and trospium are two quaternary ammonium antimuscarinic agents which have a low propensity to cross the blood/brain barrier. We hypothesized that, in combination with AChEIs, these agents would: 1) allow immediate administration of high dose AChEI therapy at effective doses, and 2) mitigate peripheral cholinergic adverse effects and improve AChEI tolerability. In this study, several fixed-dose, non-titrated combinations of donepezil combined with either glycopyrrolate or trospium were evaluated in healthy elderly subjects.

Methods: This proof-of-concept study was designed to characterize the pharmacokinetic profile, safety and tolerability of 5 mg PO donepezil plus placebo compared to 10 mg PO donepezil given concomitantly with one of three different fixed-dose regimens of either glycopyrrolate or trospium (glycopyrrolate 1 mg, glycopyrrolate 2 mg, and trospium 60 mg). Gastrointestinal tolerability was assessed at steady-state by Visual Analog Scale ratings obtained at baseline and every two hours over an eight hour observation period on day 10. Additional measures were obtained including standard laboratory values, vital signs, and pharmacokinetic profiles of donepezil, glycopyrrolate, and trospium.

Overnight polysomnography was used to measure changes in REM sleep architecture known to be affected by alterations in central cholinergic tone. Overnight polysomnography was obtained at baseline prior to initial dose, and at the end of the 10 day treatment interval. Each polysomnogram was performed on a single night, and was configured with 7 electroencephalography (EEG) channels (C3-A2, C4-A1, O1-A2, O2-A1, F3-A2, F4-A1, A1-A2) according to the 10/20 international system, two electrooculographic (EOG) leads (E1-A2, E2-A2; E1 is placed 1 cm below the left outer canthus, E2 is placed 1 cm above the right outer canthus), one chin electromyographic channel (EMG) consisting of 2 electrodes positioned submentally, with 1 cm interelectrode distance, and 1 electrocardiographic (ECG) channel (bipolar, on the chest). The sampling rate for all channels was 512 Hz. Sleep stages were determined according to the rules of the American Academy of Sleep Medicine (2007). REM sleep parameters, including REM latency, REM density and the percentage duration of REM sleep obtained in the night from Day 9 to Day 10 were compared to baseline (night from Day-1 to Day 1). Descriptive analysis for each reported variable included the number, mean, standard deviation, upper and lower limit of the 95% confidence interval of the mean, median, lower quartile, upper quartile, minimum and maximum value. Data were separately reported by dosing group and recording night. The calculation and descriptive analysis, of pairwise differences between treatment and baseline nights, for each dosing group was tested using a paired t-test and a Wilcoxon signed rank test. Differences between each of the treatment groups compared to the placebo group was performed using both a t-test for independent samples, and a Mann Whitney U-test. An overall test of treatment effect was performed using a two-way analysis of variance (ANOVA) with dosing group and time point (PSG night) as factors.

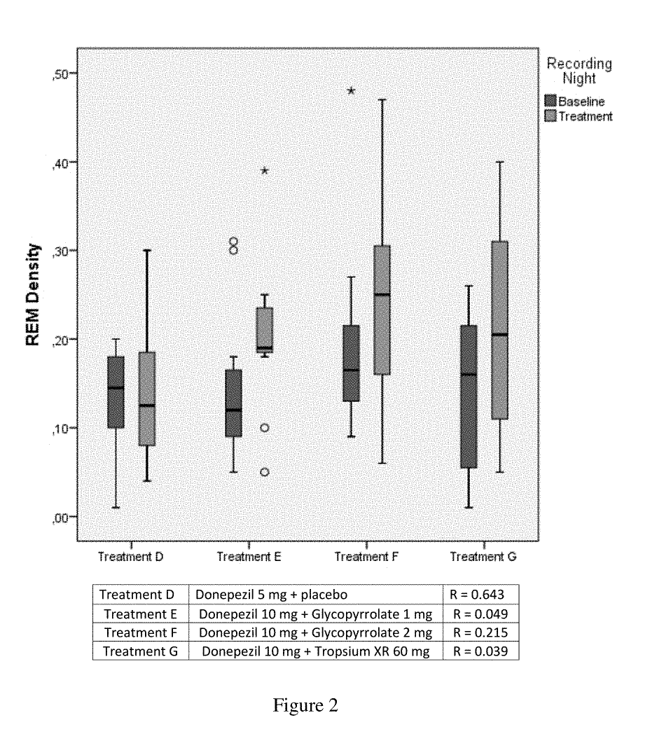

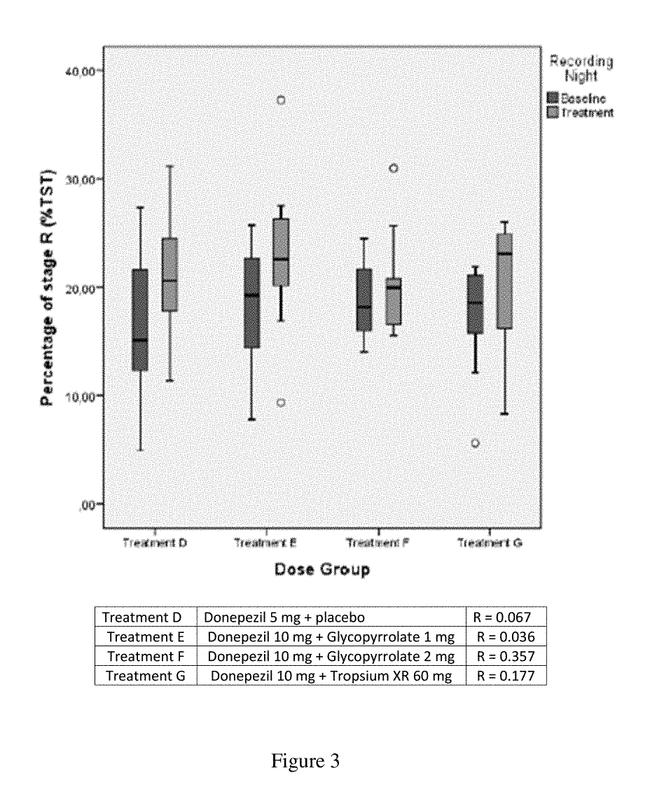

Results: At steady state, self-reported nausea using a VAS score in subjects receiving 5 mg donepezil plus placebo, achieved a peak mean change from baseline of 8.5 mm. Subjects receiving 10 mg donepezil plus treatment with a regimen containing varying dosages of either glycopyrrolate or trospium experienced a 67% to 95% reduction in nausea relative to the 5 mg donepezil plus placebo arm. Overnight polysomnography demonstrated a reduction in latency of onset of REM sleep and an increased density of REM sleep consistent with an increase in central cholinergic tone unaffected by the addition of peripheral antimuscarinic receptor blockade. FIG. 2 shows a box plot graph of the REM density data. FIG. 3 shows a box plot graph of the percentage of stage R (% TST) data.

Conclusion: The usefulness of acetylcholinesterase inhibitors (AChEIs) is limited by tolerability, mainly as a result of side effects that arise from increasing acetylcholine in the periphery (outside of the CNS). Peripherally-active anticholinergic agents have been theorized as a method of reducing undesired peripheral actions of AChEIs. These agents would be required not to cross the blood/brain barrier, which would counteract the benefits of AChEIs on the brain. Glycopyrrolate and trospium are two quaternary ammonium anticholinergic agents which have a low propensity to cross the blood/brain barrier. Sleep EEG data from a Phase 1 study in healthy subjects demonstrates an increase in percent of REM sleep and increased density of REM sleep following administration of donepezil (10 mg)+glycopyrrolate, which is consistent with an increase in cholinergic tone in the brain. These data support the view that anticholinergic agents can be given in combination with AChEIs, without impairing the increase in central cholinergic tone produced by AChEIs.

Concurrent administration of AChEIs with either of the quaternary ammonium anti-muscarinics, glycopyrrolate or trospium may: 1) allow immediate administration of high dose AChEI therapy at effective doses, and 2) mitigate peripheral cholinergic adverse effects and improve AChEI tolerability. These data also support the view that these effects can be achieved without impairing the increase in central cholinergic tone produced by AChEIs.

### Example 4: Administration of a 5-HT₆ Receptor Antagonist, an Acetylcholinesterase Inhibitor, and an Anti-Cholinergic Agent Results in Positive Outcomes for Patients with Alzheimer's Disease (Prophetic)

In this double-blind study, several fixed-dose, non-titrated combinations of 3-phenylsulfonyl-8-piperazinyl-1yl-quinoline, and donepezil, in combination with either trospium, glycopyrrolate, or solifeancin will be administered to elderly subjects previously diagnosed with mild-to-moderate Alzheimer's disease.

Qualified patients will be provided with either regimen of a sugar placebo or a combination of 35 mg 3-phenylsulfonyl-8-piperazinyl-1yl-quinoline, given concomitantly with 10 mg donepezil and either 60 mg trospium, 2 mg glycopyrrolate, or 5 mg solifenacin for two weeks. Patient cognitive ability will be assessed by the ADAS-Cog evaluation test scores obtained at baseline and at the end of the study period. Gastrointestinal tolerability will be assessed by Visual Analog Scale ratings obtained at baseline and daily. Overnight polysomnography will be used to measure changes in REM sleep architecture known to be affected by alterations in central cholinergic tone. Overnight polysomnography will be obtained at baseline prior to initial dose, and at the end of the study period. Additional measures were obtained including standard laboratory values, vital signs, and pharmacokinetic profiles of donepezil, glycopyrrolate, and trospium.

Results: We predict that patients in the treatment group will show an increase in ADAS-Cog scores compared to baseline. Additionally, patients in the treatment group will show gastrointestinal tolerability equivilant to the control group, as well as an increase in the percent of REM sleep and an increased density of REM sleep. The results will show that the combination is an effective and well-tolerated treatement for mild-to-moderate Alzheimer's disease.

### Example 5: Adminstration of a 5-HT_{2A} Receptor Inverse Agonist, an Acetylcholinesterase Inhibitor, and an Anti-Cholinergic Agent Results in Positive Outcomes for Patients with Alzheimer's Disease (Prophetic)

In this double-blind study, several fixed-dose, non-titrated combinations of 1-[3-(4-bromo-2-methyl-2H-pyrazol-3-yl)-4-methoxy-phenyl]-3-(2,4-difluoro-phenyl)-urea, and donepezil, in combination with either trospium, glycopyrrolate, or solifeancin will be administered to elderly subjects previously diagnosed with mild-to-moderate Alzheimer's disease.

Qualified patients will be provided with either regimen of a sugar placebo or a combination of 40 mg 1-[3-(4-bromo-2-methyl-2H-pyrazol-3-yl)-4-methoxy-phenyl]-3-(2,4-difluoro-phenyl)-urea, given concomitantly with 10 mg donepezil and either 60 mg trospium, 2 mg glycopyrrolate, or 5 mg solifenacin for two weeks. Patient cognitive ability will be assessed by the ADAS-Cog evaluation test scores obtained at baseline and at the end of the study period. Gastrointestinal tolerability will be assessed by Visual Analog Scale ratings obtained at baseline and daily. Overnight polysomnography will be used to measure changes in REM sleep architecture known to be affected by alterations in central cholinergic tone. Overnight polysomnography will be obtained at baseline prior to initial dose, and at the end of the study period. Additional measures were obtained including standard laboratory values, vital signs, and pharmacokinetic profiles of donepezil, glycopyrrolate, and trospium.

Results: We predict that patients in the treatment group will show an increase in ADAS-Cog scores compared to baseline. Additionally, patients in the treatment group will show gastrointestinal tolerability equivalent to the control group, as well as an increase in the percent of REM sleep and an increased density of REM sleep. The results will show that the combination is an effective and well-tolerated treatement for mild-to-moderate Alzheimer's disease.

### Example 6: Adminstration of a NMDA Receptor Antagonist, an Acetylcholinesterase Inhibitor, and an Anti-Cholinergic Agent Results in Positive Outcomes for Patients with Alzheimer's Disease (Prophetic)

In this double-blind study, several fixed-dose, non-titrated combinations of memantine and donepezil, in combination with either trospium, glycopyrrolate, or solifeancin will be administered to elderly subjects previously diagnosed with mild-to-moderate Alzheimer's disease.

Qualified patients will be provided with either regimen of a sugar placebo or a combination of 10 mg memantine, given concomitantly with 10 mg donepezil and either 60 mg trospium, 2 mg glycopyrrolate, or 5 mg solifenacin for two weeks. Patient cognitive ability will be assessed by the ADAS-Cog evaluation test scores obtained at baseline and at the end of the study period. Gastrointestinal tolerability will be assessed by Visual Analog Scale ratings obtained at baseline and daily. Overnight polysomnography will be used to measure changes in REM sleep architecture known to be affected by alterations in central cholinergic tone. Overnight polysomnography will be obtained at baseline prior to initial dose, and at the end of the study period. Additional measures were obtained including standard laboratory values, vital signs, and pharmacokinetic profiles of donepezil, glycopyrrolate, and trospium.

Results: We predict that patients in the treatment group will show an increase in ADAS-Cog scores compared to baseline. Additionally, patients in the treatment group will show gastrointestinal tolerability equivilant to the control group, as well as an increase in the percent of REM sleep and an increased density of REM sleep. The results will show that the combination is an effective and well-tolerated treatement for mild-to-moderate Alzheimer's disease.

### Example 7: Adminstration of a 5-HT₆ Receptor Antagonist, a 5-HT_{2A} Receptor Inverse Agonist, an Acetylcholinesterase Inhibitor, and an Anti-Cholinergic Agent Results in Positive Outcomes for Patients with Alzheimer's Disease (Prophetic)

In this double-blind study, several fixed-dose, non-titrated combinations of 3-phenylsulfonyl-8-piperazinyl-1yl-quinoline, 1-[3-(4-bromo-2-methyl-2H-pyrazol-3-yl)-4-methoxy-phenyl]-3-(2,4-difluoro-phenyl)-urea, and donepezil, in combination with either trospium, glycopyrrolate, or solifeancin will be administered to elderly subjects previously diagnosed with mild-to-moderate Alzheimer's disease.

Qualified patients will be provided with either regimen of a sugar placebo or a combination of 35 mg 3-phenylsulfonyl-8-piperazinyl-1yl-quinoline and 40 mg 1-[3-(4-bromo-2-methyl-2H-pyrazol-3-yl)-4-methoxy-phenyl]-3-(2,4-difluoro-phenyl)-urea, given concomitantly with 10 mg donepezil and either 60 mg trospium, 2 mg glycopyrrolate, or 5 mg solifenacin for two weeks. Patient cognitive ability will be assessed by the ADAS-Cog evaluation test scores obtained at baseline and at the end of the study period. Gastrointestinal tolerability will be assessed by Visual Analog Scale ratings obtained at baseline and daily. Overnight polysomnography will be used to measure changes in REM sleep architecture known to be affected by alterations in central cholinergic tone. Overnight polysomnography will be obtained at baseline prior to initial dose, and at the end of the study period. Additional measures were obtained including standard laboratory values, vital signs, and pharmacokinetic profiles of donepezil, glycopyrrolate, and trospium.

Results: We predict that patients in the treatment group will show an increase in ADAS-Cog scores compared to baseline. Additionally, patients in the treatment group will show gastrointestinal tolerability equivilant to the control group, as well as an increase in the percent of REM sleep and an increased density of REM sleep. The results will show that the combination is an effective and well-tolerated treatement for mild-to-moderate Alzheimer's disease.

### Example 8: Adminstration of a 5-HT₆ Receptor Antagonist, a NMDA Receptor Antagonist, an Acetylcholinesterase Inhibitor, and an Anti-Cholinergic Agent Results in Positive Outcomes for Patients with Alzheimer's Disease (Prophetic)

In this double-blind study, several fixed-dose, non-titrated combinations of 3-phenylsulfonyl-8-piperazinyl-1 yl-quinoline, memantine, and donepezil, in combination with either trospium, glycopyrrolate, or solifeancin will be administered to elderly subjects previously diagnosed with mild-to-moderate Alzheimer's disease.

Qualified patients will be provided with either regimen of a sugar placebo or a combination of 35 mg 3-phenylsulfonyl-8-piperazinyl-1yl-quinoline and 10 mg memantine, given concomitantly with 10 mg donepezil and either 60 mg trospium, 2 mg glycopyrrolate, or 5 mg solifenacin for two weeks. Patient cognitive ability will be assessed by the ADAS-Cog evaluation test scores obtained at baseline and at the end of the study period. Gastrointestinal tolerability will be assessed by Visual Analog Scale ratings obtained at baseline and daily. Overnight polysomnography will be used to measure changes in REM sleep architecture known to be affected by alterations in central cholinergic tone. Overnight polysomnography will be obtained at baseline prior to initial dose, and at the end of the study period. Additional measures were obtained including standard laboratory values, vital signs, and pharmacokinetic profiles of donepezil, glycopyrrolate, and trospium.

Results: We predict that patients in the treatment group will show an increase in ADAS-Cog scores compared to baseline. Additionally, patients in the treatment group will show gastrointestinal tolerability equivilant to the control group, as well as an increase in the percent of REM sleep and an increased density of REM sleep. The results will show that the combination is an effective and well-tolerated treatement for mild-to-moderate Alzheimer's disease.

### Example 9: Adminstration of a 5-HT_{2A} Receptor Inverse Agonist, a NMDA Receptor Antagonist, an Acetylcholinesterase Inhibitor, and an Anti-Cholinergic Agent Results in Positive Outcomes for Patients with Alzheimer's Disease (Prophetic)

In this double-blind study, several fixed-dose, non-titrated combinations of 1-[3-(4-bromo-2-methyl-2H-pyrazol-3-yl)-4-methoxy-phenyl]-3-(2,4-difluoro-phenyl)-urea, memantine, and donepezil, in combination with either trospium, glycopyrrolate, or solifeancin will be administered to elderly subjects previously diagnosed with mild-to-moderate Alzheimer's disease.

Qualified patients will be provided with either regimen of a sugar placebo or a combination of 40 mg 1-[3-(4-bromo-2-methyl-2H-pyrazol-3-yl)-4-methoxy-phenyl]-3-(2,4-difluoro-phenyl)-urea and 10 mg memantine, given concomitantly with 10 mg donepezil and either 60 mg trospium, 2 mg glycopyrrolate, or 5 mg solifenacin for two weeks. Patient cognitive ability will be assessed by the ADAS-Cog evaluation test scores obtained at baseline and at the end of the study period. Gastrointestinal tolerability will be assessed by Visual Analog Scale ratings obtained at baseline and daily. Overnight polysomnography will be used to measure changes in REM sleep architecture known to be affected by alterations in central cholinergic tone. Overnight polysomnography will be obtained at baseline prior to initial dose, and at the end of the study period. Additional measures were obtained including standard laboratory values, vital signs, and pharmacokinetic profiles of donepezil, glycopyrrolate, and trospium.

Results: We predict that patients in the treatment group will show an increase in ADAS-Cog scores compared to baseline. Additionally, patients in the treatment group will show gastrointestinal tolerability equivilant to the control group, as well as an increase in the percent of REM sleep and an increased density of REM sleep. The results will show that the combination is an effective and well-tolerated treatement for mild-to-moderate Alzheimer's disease.

### Example 10: Adminstration of a 5-HT₆ Receptor Antagonist, a 5-HT_{2A} Receptor Inverse Agonist, a NMDA Receptor Antagonist, an Acetylcholinesterase Inhibitor, and an Anti-Cholinergic Agent Results in Positive Outcomes for Patients with Alzheimer's Disease (Prophetic)

In this double-blind study, several fixed-dose, non-titrated combinations of 3-phenylsulfonyl-8-piperazinyl-1yl-quinoline, 1-[3-(4-bromo-2-methyl-2H-pyrazol-3-yl)-4-methoxy-phenyl]-3-(2,4-difluoro-phenyl)-urea, memantine, and donepezil, in combination with either trospium, glycopyrrolate, or solifeancin will be administered to elderly subjects previously diagnosed with mild-to-moderate Alzheimer's disease.

Qualified patients will be provided with either regimen of a sugar placebo or a combination of 35 mg 3-phenylsulfonyl-8-piperazinyl-1yl-quinoline, 40 mg 1-[3-(4-bromo-2-methyl-2H-pyrazol-3-yl)-4-methoxy-phenyl]-3-(2,4-difluoro-phenyl)-urea and 10 mg memantine, given concomitantly with 10 mg donepezil and either 60 mg trospium, 2 mg glycopyrrolate, or 5 mg solifenacin for two weeks. Patient cognitive ability will be assessed by the ADAS-Cog evaluation test scores obtained at baseline and at the end of the study period. Gastrointestinal tolerability will be assessed by Visual Analog Scale ratings obtained at baseline and daily. Overnight polysomnography will be used to measure changes in REM sleep architecture known to be affected by alterations in central cholinergic tone. Overnight polysomnography will be obtained at baseline prior to initial dose, and at the end of the study period. Additional measures were obtained including standard laboratory values, vital signs, and pharmacokinetic profiles of donepezil, glycopyrrolate, and trospium.

Results: We predict that patients in the treatment group will show an increase in ADAS-Cog scores compared to baseline. Additionally, patients in the treatment group will show gastrointestinal tolerability equivilant to the control group, as well as an increase in the percent of REM sleep and an increased density of REM sleep. The results will show that the combination is an effective and well-tolerated treatement for mild-to-moderate Alzheimer's disease.

Although the present disclosure has been described in considerable detail with reference to certain preferred versions thereof, other versions are possible. Therefore, the spirit and scope of the application should not be limited to the description of the preferred versions described herein.

Although compositions, materials, and methods similar or equivalent to those described herein can be used in the practice or testing of the present disclosure, suitable preparations, methods and materials are described herein. All publications mentioned herein are incorporated by reference in their entirety. In the case of conflict, the present specification, including definitions will control. In addition, the particular embodiments discussed below are illustrative only and not intended to be limiting.

All features disclosed in the specification, including the abstract and drawings, and all the steps in any method or process disclosed, may be combined in any combination, except combinations where at least some of such features and/or steps are mutually exclusive. Each feature disclosed in the specification, including abstract and drawings, can be replaced by alternative features serving the same, equivalent or similar purpose, unless expressly stated otherwise. Thus, unless expressly stated otherwise, each feature disclosed is one example only of a generic series of equivalent or similar features. Various modifications of the application, in addition to those described herein, will be apparent to those skilled in the art from the foregoing description. Such modifications are also intended to fall within the scope of the appended claims.

Unless otherwise indicated, all numbers expressing quantities of ingredients, properties such as molecular weight, reaction conditions, and so forth used in the specification and claims are to be understood as being modified in all instances by the term “about.” As used herein, the term “about” means plus or minus 10% of a given value. For example, “about 50%” means in the range of 45%-55%. Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein may be further limited in the claims using “consisting of” or “consisting essentially of” language, rather than “comprising”. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

In closing, it is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

What is claimed is:

1. A composition comprising:
a therapeutically effective amount of 3-phenylsulfonyl-8-piperazinyl-1yl-quinoline or pharmaceutically acceptable salts, hydrates or solvates thereof;
a therapeutically effective amount of an NMDA receptor antagonist;
a therapeutically effective amount of an acetylcholinesterase inhibitor;
a therapeutically effective amount of an anti-cholinergic agent; and
at least one pharmaceutically acceptable excipient.

2. The composition of claim 1, wherein the composition is suitable for oral administration.

3. The composition of claim 1, wherein the therapeutically effective amount of 3-phenylsulfonyl-8-piperazinyl-1 yl-quinoline or pharmaceutically acceptable salts, hydrates, polymorphs, or solvates thereof is from about 0.001 mg to about 1,000 mg, about 0.001 mg to about 200 mg, about 0.001 mg to about 175 mg, or 0.001 mg to about 70 mg.

4. The composition of claim 1, wherein the therapeutically effective amount of 3-phenylsulfonyl-8-piperazinyl-1 yl-quinoline or pharmaceutically acceptable salts, hydrates, polymorphs, or solvates thereof is about 15 mg, about 35 mg, or about 70 mg.

5. The composition of claim 1, wherein the therapeutically effective amount of 3-phenylsulfonyl-8-piperazinyl-1 yl-quinoline or pharmaceutically acceptable salts, hydrates, polymorphs, or solvates thereof is an amount selected from the group consisting of an amount of 3-phenylsulfonyl-8-piperazinyl-1yl-quinoline that may cause convulsions in a subject to which it is administered; an amount that would be expected to exceed the maximum tolerated dose for the subject to which it is administered; an amount associated with systemic exposures characterized by an AUCₜₐᵤ₋ₛₛ of about 8.2 μg·h/ml, a Cₘₐₓ of about 0.26 μg/ml; or a combination thereof a mount associated with systemic exposures characterized by an AUC, Cₘₐₓ, or combinations thereof, that are about 2 to about 3 times higher than the mean clinical exposure achieved at the proposed clinical dose for monotherapy with 3-phenylsulfonyl-8-piperazinyl-1yl-quinoline (i.e. mean AUCₜₐᵤ₋ₛₛ of about 3.2 μg·h/ml and Cₘₐₓ of about 0.180 μg/ml), an amount associated with a recorded systemic clinical exposure that is greater than the highest recorded systemic clinical exposure (AUC_{0-∞} of about 9.25 μg·h/ml and Cₘₐₓ of about 0.293 an amount of 3-phenylsulfonyl-8-piperazinyl-1yl-quinoline that is greater than about 10 mg/kg/day, an amount of 3-phenylsulfonyl-8-piperazinyl-1yl-quinoline that is greater than 15 mg/day, a dose of 3-phenylsulfonyl-8-piperazinyl-1yl-quinoline that is greater than about 35 mg/day or any combination thereof.

6. The composition of claim 1, wherein the NMDA receptor antagonist is selected from the group consisting of memantine, amantadine, and ketamine.

7. The composition of claim 6, wherein the NMDA receptor antagonist is memantine or pharmaceutically acceptable salts, hydrates, polymorphs, or solvates thereof.

8. The composition of claim 7, wherein the therapeutically effective amount of memantine or pharmaceutically acceptable salts, hydrates, polymorphs, or solvates thereof is from about 0.001 mg to about 1,000 mg, or about 0.001 mg to about 30 mg.

9. The composition of claim 7, wherein the therapeutically effective amount of memantine or pharmaceutically acceptable salts, hydrates, polymorphs, or solvates thereof is about 5 mg, about 7 mg, about 10 mg, about 14 mg, about 20 mg, about 21 mg, or about 28 mg.

10. The composition of claim 1, wherein the acetylcholinesterase inhibitor is selected from the group consisting of donepezil, rivastigmine, galantamine, tacrine, physostigmine, pyridostigmine, neostigmine, icopezil, zanapezil, ipidacrine, phenserine, ambenonium, edrophonium, ladostigil, huperzine A, or pharmaceutically acceptable salts, hydrates, polymorphs, or solvates thereof.

11. The composition of claim 10, wherein the acetylcholinesterase inhibitor is donepezil or pharmaceutically acceptable salts, hydrates, polymorphs, or solvates thereof.

12. The composition of claim 11, wherein the therapeutically effective amount of donepezil or pharmaceutically acceptable salts, hydrates, polymorphs, or solvates thereof is configured for immediate release, extended release, delayed release, or any combination thereof.

13. The composition of claim 11, wherein the therapeutically effective amount of donepezil or pharmaceutically acceptable salts, hydrates, polymorphs, or solvates thereof is from about 0.001 mg to about 1,000 mg, about 0.001 mg to about 30 mg, or about 0.2 mg to about 138 mg.

14. The composition of claim 11, wherein the therapeutically effective amount of donepezil or pharmaceutically acceptable salts, hydrates, polymorphs, or solvates thereof is about 5 mg, 10 mg, or 23 mg.

15. The composition of claim 1, wherein the anti-cholinergic agent is selected from the group consisting of quaternary ammonium anti-cholinergic muscarinic receptor antagonist, a quaternary ammonium non-selective peripheral Anti-Cholinergic agent, a sulfonium non-selective peripheral Anti-Cholinergic agent, a non-selective peripheral muscarinic anti-cholinergic agent, (1S)-(3R)-1-azabicyclo[2.2.2]oct-3-yl 3,4-dihydro-1-phenyl-2(1H)-iso-quinolinecarboxylate (solifenacin) and its pharmaceutically acceptable salts, 1-methylpiperidin-4-yl) 2,2-di(phenyl)-2-propoxyacetate (propiverine) and its pharmaceutically acceptable salts, 1,4,5,6-tetrahydro-1-methylpyrimidin-2-ylmethyl α-cyclohexyl-α-hydroxy-α-phenylacetate (oxyphencyclimine) and its pharmaceutically acceptable salts, (R)-N,N-diisopropyl-3-(2-hydroxy-5-methylphenyl)-3-phenylpropanamine (tolterodine) and its pharmaceutically acceptable salts.

16. The composition of claim 15, wherein the quaternary ammonium anti-cholinergic muscarinic receptor antagonist is selected from trospium and glycopyrrolate or pharmaceutically acceptable salts, hydrates or solvates thereof.

17. The composition of claim 15, wherein the quaternary ammonium anti-cholinergic muscarinic receptor antagonist is trospium or pharmaceutically acceptable salts, hydrates or solvates thereof.

18. The composition of claim 17, wherein the quaternary ammonium anti-cholinergic muscarinic receptor antagonist is trospium chloride.

19. The composition of claim 17, wherein the therapeutically effective amount of trospium or pharmaceutically acceptable salts, hydrates or solvates thereof is from about 0.1 mg to about 120 mg.

20. The composition of claim 17, wherein the therapeutically effective amount of trospium or pharmaceutically acceptable salts, hydrates or solvates thereof is about 20 mg, about 40 mg, or about 60 mg.

21. The composition of claim 17, wherein the therapeutically effective amount of glycopyrrolate is an amount from about 20% to about 600% of the amount of trospium or pharmaceutically acceptable salts, hydrates or solvates thereof that is currently administered for anti-cholinergic therapy.

22. A method of treating a neurodegenerative disease in a patient in need thereof comprising administering the patient a composition of any of claims 1-21.

23. The method of claim 22, wherein the neurodegenerative disease is selected from the group consisting of Pick's disease, Fronto-temporal dementia, Progressive Supranuclear Palsy Alzheimer's disease (including mild or early-stage Alzheimer's disease, mild to moderate Alzheimer's disease, moderate or mid-stage Alzheimer's disease, moderate to severe Alzheimer's disease, moderately severe Alzheimer's disease, severe Alzheimer's disease, Alzheimer's disease with Lewy bodies, (AD)), Parkinson's disease (including Parkinson's disease chemically induced by exposure to environmental agents such as pesticides, insecticides, or herbicides and/or metals such as manganese, aluminum, cadmium, copper, or zinc, SNCA gene-linked Parkinson's disease, sporadic or idiopathic Parkinson's disease, or Parkin- or LRRK2-linked Parkinson's disease (PD)), autosomal-dominant Parkinson's disease, Diffuse Lewy Body Disease (DLBD) also known as Dementia with Lewy Bodies (DLB), Pure Autonomic Failure, Lewy body dysphagia, Incidental LBD, Inherited LBD (e.g., mutations of the alpha-synuclein gene, PARK3 and PARK4), multiple system atrophy (including Olivopontocerebellar Atrophy, Striatonigral Degeneration, Shy-Drager Syndrome (MSA)), combined Alzheimer's and Parkinson disease and/or MSA, Huntington's disease, synucleinopathies, disorders or conditions characterized by the presence of Lewy bodies, multiple sclerosis, Amyotrophic lateral sclerosis (ALS) dementia (including vascular dementia, Lewy body dementia, Parkinson's dementia, frontotemporal dementia), Down syndrome, Psychosis (including agitation caused by a neurodegenerative disease or associated with dopaminergic therapy such as but not limited to Parkinson's disease psychosis, Alzheimer's disease psychosis, Lewy body dementia psychosis), dyskinesia (including agitation caused by a neurodegenerative disease or associated with dopaminergic therapy), agitation (including agitation caused by a neurodegenerative disease or associated with dopaminergic therapy), conditions associated with dopaminergic therapy (including dystonia, myoclonus, or tremor), synucleinopathies, diseases, disorders or conditions associated with abnormal expression, stability, activities and/or cellular processing of α-synuclein, diseases, disorders or conditions characterized by the presence of Lewy bodies, and combinations thereof.

24. A method for treating a neurodegenerative disease in a patient in need thereof comprising administering to the patient a therapeutically effective amount of 3-phenylsulfonyl-8-piperazinyl-1yl-quinoline or pharmaceutically acceptable salts, hydrates or solvates thereof; a therapeutically effective amount of memantine or pharmaceutically acceptable salts, hydrates or solvates thereof; a therapeutically effective amount of trospium or pharmaceutically acceptable salts, hydrates or solvates thereof; and a therapeutically effective amount of donepezil or pharmaceutically acceptable salts, hydrates or solvates thereof.

25. The method of claim 24, wherein the a neurodegenerative disease is selected from the group consisting of Pick's disease, Fronto-temporal dementia, Progressive Supranuclear Palsy Alzheimer's disease (including mild or early-stage Alzheimer's disease, mild to moderate Alzheimer's disease, moderate or mid-stage Alzheimer's disease, moderate to severe Alzheimer's disease, moderately severe Alzheimer's disease, severe Alzheimer's disease, Alzheimer's disease with Lewy bodies, (AD)), Parkinson's disease (including Parkinson's disease chemically induced by exposure to environmental agents such as pesticides, insecticides, or herbicides and/or metals such as manganese, aluminum, cadmium, copper, or zinc, SNCA gene-linked Parkinson's disease, sporadic or idiopathic Parkinson's disease, or Parkin- or LRRK2-linked Parkinson's disease (PD)), autosomal-dominant Parkinson's disease, Diffuse Lewy Body Disease (DLBD) also known as Dementia with Lewy Bodies (DLB), Pure Autonomic Failure, Lewy body dysphagia, Incidental LBD, Inherited LBD (e.g., mutations of the alpha-synuclein gene, PARK3 and PARK4), multiple system atrophy (including Olivopontocerebellar Atrophy, Striatonigral Degeneration, Shy-Drager Syndrome (MSA)), combined Alzheimer's and Parkinson disease and/or MSA, Huntington's disease, synucleinopathies, disorders or conditions characterized by the presence of Lewy bodies, multiple sclerosis, Amyotrophic lateral sclerosis (ALS) dementia (including vascular dementia, Lewy body dementia, Parkinson's dementia, frontotemporal dementia), Down syndrome, Psychosis (including agitation caused by a neurodegenerative disease or associated with dopaminergic therapy such as but not limited to Parkinson's disease psychosis, Alzheimer's disease psychosis, Lewy body dementia psychosis), dyskinesia (including agitation caused by a neurodegenerative disease or associated with dopaminergic therapy), agitation (including agitation caused by a neurodegenerative disease or associated with dopaminergic therapy), conditions associated with dopaminergic therapy (including dystonia, myoclonus, or tremor), synucleinopathies, diseases, disorders or conditions associated with abnormal expression, stability, activities and/or cellular processing of α-synuclein, diseases, disorders or conditions characterized by the presence of Lewy bodies, and combinations thereof.

26. The method of claim 24, wherein the therapeutically effective amount of 3-phenylsulfonyl-8-piperazinyl-1yl-quinoline or pharmaceutically acceptable salts, hydrates, polymorphs, or solvates thereof is about 15 mg, about 35 mg, or about 70 mg.

27. The method of claim 24, wherein the therapeutically effective amount of memantine or pharmaceutically acceptable salts, hydrates, polymorphs, or solvates thereof is about 5 mg, about 7 mg, about 10 mg, about 14 mg, about 20 mg, about 21 mg, or about 28 mg.

28. The method of claim 24, comprising administering about 5 mg/day, 10 mg/day, or 23 mg/day of donepezil or pharmaceutically acceptable salts, hydrates or solvates thereof to the patient.

29. The method of claim 24, comprising administering from about 0.1 to about 120 mg/day of trospium or pharmaceutically acceptable salts, hydrates or solvates thereof to the patient.

30. The method of claim 24, comprising administering about 0.1 mg/day to about 120 mg/day of trospium or pharmaceutically acceptable salts, hydrates or solvates thereof to the patient and between about 5 mg/day and about 23 mg/day of donepezil to the patient.

31. The method of claim 24, comprising administering about 10 mg/day of donepezil or pharmaceutically acceptable salts, hydrates or solvates thereof to the patient to the patient.

32. The method of claim 24, comprising administering about 60 mg/day of trospium or pharmaceutically acceptable salts, hydrates or solvates thereof to the patient to the patient
